(19) United States
(12) Patent Application Publication
(10) Pub. No.: US 2024/0355219 A1
(43) Pub. Date: Oct. 24, 2024

(54) SYSTEMS AND METHODS FOR SIMULATING SURGICAL PROCEDURES

(71) Applicant: FVRVS Limited, London (GB)

(72) Inventors: Richard Vincent, London (GB); Victoria Jane Smalley, London (GB)

(21) Appl. No.: 18/618,368

(22) Filed: Mar. 27, 2024

Related U.S. Application Data
(60) Provisional application No. 63/455,055, filed on Mar. 28, 2023.
(60) Provisional application No. 63/455,057, filed on Mar. 28, 2023.
(60) Provisional application No. 63/455,062, filed on Mar. 28, 2023.
(60) Provisional application No. 63/455,063, filed on Mar. 28, 2023.
(60) Provisional application No. 63/455,064, filed on Mar. 28, 2023.
(60) Provisional application No. 63/455,067, filed on Mar. 28, 2023.

(51) Int. Cl. G09B 9/00; A61B 34/00; A61B 34/10; G06F 3/04815; G06F 3/0484

(52) U.S. Cl. CPC G09B 9/00; A61B 34/76; G06F 3/04815; G06F 3/0484; A61B 2034/104; A61B 2034/105

(57) ABSTRACT

Systems of the invention include a platform that provides a suite of features delivering scalable, cost-effective, and realistic simulations that may be integrated into a surgical training curriculum. In particular, the invention provides for multi-user interaction within a hardware/software agnostic platform that addresses the technical issues related to the realism and accuracy necessary for effective use of simulated surgical procedures as a training tool. As such, systems and methods of the invention provide for dynamic, frictionless remote collaboration, unlimited repetition, and assessment data insight on capability and risk, which reduces costs and improves patient outcomes.

### CROSS-REFERENCE TO RELATED APPLICATIONS

This application claims priority to, and the benefit of, U.S. Provisional Application No. 63/455,055, filed Mar. 28, 2023, U.S. Provisional Application No. 63/455,057, filed Mar. 28, 2023, U.S. Provisional Application No. 63/455,062, filed Mar. 28, 2023, U.S. Provisional Application No. 63/455,063, filed Mar. 28, 2023, U.S. Provisional Application No. 63/455,064, filed Mar. 28, 2023, and U.S. Provisional Application No. 63/455,067, filed Mar. 28, 2023, the contents of each of which are incorporated by reference herein in their entirety.

### FIELD OF THE INVENTION

The invention relates to systems and methods for providing simulated surgical procedures.

### BACKGROUND

Surgical training for doctors is a complex and lengthy process. Currently, surgeons must spend years in training as a surgical resident or fellow because the learning curve is so long. Typically, an apprentice surgeon only masters a surgical procedure after having done it tens or hundreds of times, and the surgical outcome can be less than optimal for the surgeries performed by an apprentice surgeon.

Despite intentions to move towards competency-based educational models to train the modern surgeon, there is no agreement on how to best prepare educators to train the new generation of surgeons. Lectures, passive watching of procedures, and accumulated hours of passive professional experience have proven ineffective as learning methods. Further, there is concern that regulations, patient safety issues, and public opinions have led to decreased responsibility and autonomy among residents, which may lead to surgeons being unable to perform independently after completed training.

It is estimated that up to half of all major complications from surgical procedures are potentially avoidable, further highlighting the need to develop evaluation methods to ensure that the current practice is successful in producing surgeons competent in technical skills in a number sufficient to meet global surgery needs. Additionally, it has been found that a good portion of surgical errors involve failures in communication among personnel. Thus, health care providers and hospitals have an ongoing need for training and skills development and for new and innovative ways to economically deliver skills transfer.

Simulated training systems, including virtual reality (VR) and/or augmented reality (AR) technologies, are advanced systems that can provide an immersive testbed for the training and application of theoretical knowledge. Accordingly, such technologies present the opportunity to provide a low-risk and cost effective means for surgical training.

While high-quality VR and AR experiences and simulations are currently available, the application of such technologies in simulating high precision real-world scenarios within the surgical field remains a challenge. Specifically, current systems and the advanced technology required for them have drawbacks preventing VR/AR platforms from being widely adopted.

In particular, significant challenges relate to the realism and accuracy of the simulation, with many systems simply providing screen-based animated textbooks or, at best, a three-dimensional (3D) walkthrough of a given procedure. Furthermore, many simulation systems are specific to a certain procedure, resulting in systems that are rigidly defined and offer little to no flexibility and/or adaptability. As a result, system tasks may be overly simplified and unrealistic with regard to real-world procedures and scenarios, and thus offer limited experiences. Other drawbacks include inconsistent and poor technical performance, as well as the prohibitively high cost of such systems, in that certain platforms require specialized hardware and/or software and have associated space limitations. As a result, existing simulation systems remain unable to provide users with a convincing and immersive training experience such that true skills transfer is hindered.

Finally, any assessment metrics that may be used in current simulation systems lack clinical significance. In particular, while such metrics may involve measuring certain performance characteristics, such measurements are performed without considering the relationship of the performance characteristics to any required real-world skills. Accordingly, it is difficult to determine the correspondence between different training approaches due, in part, to the unavailability of uniform tests or reporting schemes. Additional challenges relate to attempts to introduce rigorous evaluation of simulation reliability and validity, as well as the integration of surgical simulation into medical educational curriculum. Accordingly, such challenges prevent the incorporation of surgical simulation technologies in medical education curriculum. Although VR/AR simulation technologies may offer an answer to the challenges related to training the next generation of surgeons, there exists a need for improved systems.

### SUMMARY

The present invention recognizes the drawbacks of current virtual reality (VR), augmented reality (AR), mixed reality (MR), and cross-reality (XR) surgical simulation systems, namely the technical challenges that limit the use of these systems. The invention solves these problems by providing systems and methods providing realistic, immersive, multi-user, and collaborative simulated surgical environments for surgical training, rehearsal, and assessments, such that pre-human competence may be achieved.

Pre-human competence is the competence obtained in the simulated surgical and/or medical environment as applied to a virtual patient. Accordingly, the realistic simulated surgical environment experience, with rehearsal and assessment in a team environment, may be used to obtain competence such that the skills obtained may be transferred to a real-world medical procedure and patient. Accordingly, the invention allows for scalable, cost-effective, and authentic simulations that are capable of integration into a surgical training curriculum.

In particular, the invention provides a simulation system with a novel infrastructure that combines a mix of haptic and non-haptic VR, AR, MR, and/or XR experiences at scale. The invention provides enhanced technical fidelity with accurate deformation models and soft tissue animation, that incorporate precise material properties for a convincing immersion experience.

As described in detail herein, the invention accomplishes highly realistic, real-time, free-form simulations of surgical procedures by combining three simulation disciplines—physical, visual, and haptic—in novel ways by utilizing a system incorporating various engines capable of providing a high level of user immersion. The system is built upon improved techniques using voxel representation, position-based dynamics, and solid constructive geometry to generate simulations that exhibit emergent properties and give infinite variability in outcome. Thus, the simulation experience is fluid and natural and under the direct control of the user. For example, the platform allows for users to decide how to interact with the tissues of a virtual patient, such as choosing where to cut, where to resect, and/or suture, and seeing and feeling the direct impact of these decisions. This is accomplished in a generalized way without the need for custom hardware.

The systems, via associated platforms, may include a surgical haptic intelligence engine, and/or a simulation engine, with unique processing techniques for creating visual and haptic textures that provide a fully realistic computer simulated surgery experience. Dynamic haptic actions and interactions made possible by the systems of the invention provide a seamless and realistic VR/AR/XR operating room experience wherein the user is able to engage in a fully realistic computer simulated surgery and simulated surgical environment.

The systems combine visual, audible, and haptic capabilities and specific features for full procedural training and rehearsal with surgical skills assessment, team training, remote proctoring/monitoring, and capability assessment, with particular focus on precision skills transfer.

For example, systems of the invention include multi-user simulated surgical environments with live to VR capabilities. This allows for remote collaboration that may be integrated into the live operating room. The novel infrastructure delivers simulated surgical experiences that allow teams to remotely collaborate and to rehearse procedures by physically interacting with the virtual patient within the simulated surgical environment.

The invention provides systems for developing and simulating patient-specific and specific patient procedures, for example, for virtual surgical simulation of a specific case and/or before a specific patient's procedure. The system provides digital content developed from data obtained from various data sources related to, for example, a specific case and/or specific patient. As an example, the system uses data from various sources for a particular patient case to create a simulation that mirrors that particular case. Additionally, the system may use data from a specific patient before a case is conducted to simulate a procedure related to the specific patient such that the procedure may be rehearsed before the actual surgery.

Further, the invention includes systems and methods for real-time simulation of complex soft tissue interactions and more complex behaviors such as bleeding and contractions, soft tissue suturing, and knot-tying. The invention also provides for creating digital twins for full replication of physical tools and devices that may be used for testing and validation. The invention also provides systems and methods for virtual imaging in the simulated surgical environment.

Accordingly, the systems and methods of the invention disclosed herein provide for realistic precision training on surgical techniques, frictionless remote collaboration, unlimited repetition, and assessment data insight on capability and risk, all of which reduce overall costs and improve patient outcomes.

### Multi-User Simulated Surgical Environment

Aspects of the invention includes a system for providing a simulated surgical environment that includes a simulation platform a simulation platform configured to communicate and exchange data with one or more hand-held components and one or more wearable displays over a network. The platform comprises a non-transitory computer-readable storage medium coupled to the processor and encoded with a computer program that causes the processor to generate and provide digital content comprising one or more simulated objects within a simulated surgical environment to be presented to multiple users interacting with the simulated surgical environment via associated hand-held components and wearable displays. The associated hand-held components and wearable displays are configured to provide haptic and visual feedback, respectively, to each of the multiple users within the simulated surgical environment. The platform causes the processor to monitor actions of the multiple users to identify at least one of a user's field of view within the surgical environment and user interaction with a simulated object within the surgical environment based on physical user interaction with an associated hand-held component. The processor then adjusts adjust output and synchronizes sharing of digital content in real time across at least one of the hand-held component and wearable display associated with a given user in response to the actions of one or more of the users to thereby allow the multiple users to simultaneously interact within the simulated surgical environment via the hand-held components and wearable displays.

In some embodiments of the system, the simulation platform is configured to determine a type and a degree of haptic feedback to be provided to one or more users via an associated hand-held component based on a user's physical interaction with an associated hand-held component and the type of simulated objected. For example, in some embodiments, the simulation platform is configured to adjust output of digital content to thereby mimic operation and/or movement of the simulated objected. In some embodiments, the simulated object is a simulated surgical instrument.

In some embodiments of the system, the simulation platform is configured to allow for the one or more users to touch, manipulate, and/or modify the simulated object within the simulated surgical environment via an associated hand-held component. The simulation platform may be further configured to track actions of the one or more users; assess a performance of the one or more users based on the tracked actions; determine a performance rating for the actions of the user; and provide feedback to the one or more users associated with their performance rating. For example, in some embodiments, the actions of a first user are transmitted to the wearable display and the hand-held device of one or more other users.

In some embodiments of the system, the data communicated and exchanged between the simulation platform and the one or more hand-held components and one or more wearable displays includes at least one of behavioral telemetry data, decision data, and performance outcome data associated with a user performing a surgical procedure in a live operating room. For example, in some embodiments, the simulated object comprises augmented reality (AR) content to be displayed as an overlay on a view of a real object in the live operating room.

In some embodiments of the system, the haptic and/or visual feedback is computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined.

In another aspect, the invention discloses a method for providing a simulated surgical environment. The method includes the steps of generating and providing, via a simulation platform, digital content comprising one or more simulated objects within a simulated surgical environment to be presented to multiple users interacting with the simulated surgical environment via associated hand-held components and wearable displays. The associated hand-held components and wearable displays are configured to provide haptic and visual feedback, respectively, to each of the multiple users within the simulated surgical environment. The method also includes monitoring, via the simulation platform, actions of the multiple users to identify at least one of a user's field of view within the surgical environment and user interaction with a simulated object within the surgical environment based on physical user interaction with an associated hand-held component. Further, the method includes adjusting output and synchronizing sharing of digital content, via the simulation platform, in real time across at least one of the hand-held component and wearable display associated with a given user in response to the actions of one or more of the users to thereby allow the multiple users to simultaneously interact within the simulated surgical environment via the hand-held components and wearable displays.

In some embodiments of the method, the method includes determining, via the simulation platform, a type and a degree of haptic feedback to be provided to one or more users via an associated hand-held component based on a user's physical interaction with an associated hand-held component and the type of simulated objected. The output of digital content may be adjusted to thereby mimic operation and/or movement of the simulated objected. In some embodiments, the simulated object is a simulated surgical instrument.

In some embodiments of the method, the simulation platform is configured to allow for the one or more users to touch, manipulate, and/or modify the simulated object within the simulated surgical environment via an associated hand-held component. The simulation platform may be, in some embodiments, further configured to track actions of the one or more users; assess a performance of the one or more users based on the tracked actions; determine a performance rating for the actions of the user; and provide feedback to the one or more users associated with their performance rating. Further, actions of a first user are transmitted to the wearable display and the hand-held device of one or more other users. In some embodiments, the simulation platform is configured to communicate and exchange data with the hand-held components and wearable displays over a network, wherein the data comprises one or more of behavioral telemetry data, decision data, and performance outcome data associated with a user performing a surgical procedure in a live operating room. For example, in some embodiments, the simulated object comprises augmented reality (AR) content to be displayed as an overlay on a view of a real object in a live operating room.

In some embodiments of the method, the haptic and/or visual feedback is computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined.

### Patient-Specific Surgical Simulation

Aspects of the invention include systems for providing a patient-specific simulated surgery. The system includes a simulation platform configured to communicate and exchange data with one or more hand-held components and one or more wearable displays over a network. The platform includes a non-transitory computer-readable storage medium coupled to the processor and encoded with a computer program that causes the processor to receive data of a patient that represents a completed, real world surgical procedure of the patient; and generate and provide digital content comprising one or more simulated objects within a simulated surgical environment associated with the real world surgical procedure undergone by the patient, which is presented to one or more users interacting with the simulated surgical environment via associated hand-held components and wearable displays. The simulated content is based, at least in part, on the data received from the completed, real-world surgical procedure of the patient.

In some embodiments, the simulation platform is configured to monitor user actions within the simulated surgical environment, for example where the user actions impact the outcome of the simulated surgical procedure as compared to the outcome of the real-world surgical procedure. In this way, systems of the invention can track the user(s) actions as compared to the real-world procedure.

In some embodiments, the digital content comprises at least one of virtual reality (VR) content and augmented reality (AR) content. For example, in some embodiments, the digital content is a virtual three-dimensional (3D) model of a patient to undergo a surgical procedure.

The digital content, in some embodiments, is based, at least in part, on data received via a standard protocol for the management and transmission of real medical images and related data; real behavioral telemetry data from a surgical procedure; real performance outcome data from a surgical procedure; and/or real decision data from a surgical procedure. In some embodiments, the standard protocol is Digital Imaging and Communications in Medicine (DICOM).

In some embodiments, the simulation platform further includes a simulation engine configured to create the digital content for the simulated surgical procedure. In particular, in some embodiments, the simulation platform is configured to receive scan data related to one or more surgical scenarios; segment the scan data; apply anatomical landmarks; apply one or more haptic values to the data; apply a templating algorithm to generate a 3D model of an anatomy; and parameterize the anatomy to create a simulated patient requiring a surgical procedure.

In some embodiments, the system is configured to monitor actions of one or more users to identify at least one of a user's field of view within the surgical environment and user interaction with a simulated object within the surgical environment based on physical user interaction with an associated hand-held component. The system is configured to the adjust output of digital content across at least one of the hand-held component and wearable display associated with a given user in response to the actions of one or more of the users.

Further, the haptic and/or visual feedback to be provided to a given user via an associated hand-held component and wearable display is computed, in some embodiments, using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment. Accordingly, the algorithm establishes a resultant effect of the interaction with the digital content that is not pre-determined.

In another aspect, the invention includes a method for providing a patient-specific surgical environment. The method includes receiving, via a simulation platform, data of a patient that represents a completed, real world surgical procedure of the patient; and generating and providing, via the simulation platform, digital content comprising one or more simulated objects within a simulated surgical environment associated with the real world surgical procedure undergone by the patient, which is presented to one or more users interacting with the simulated surgical environment via associated hand-held components and wearable displays. The simulated content is based, at least in part, on the data received from the completed, real-world surgical procedure of the patient.

In some embodiments of the method, the simulation platform is configured to monitor user actions within the simulated surgical environment, wherein said user actions impact the outcome of the simulated surgical procedure as compared to the outcome of the real-world surgical procedure.

In some embodiments of the methods, the digital content comprises at least one of virtual reality (VR) content and augmented reality (AR) content. For example, in some embodiments, the digital content includes a virtual three-dimensional (3D) model of a patient to undergo a surgical procedure.

In some embodiments of methods of the invention, the digital content is based, at least in part, on data received via a standard protocol for the management and transmission of real medical images and related data; real behavioral telemetry data from a surgical procedure; real performance outcome data from a surgical procedure; and/or real decision data from a surgical procedure. In specific embodiments, the standard protocol is Digital Imaging and Communications in Medicine (DICOM).

Further, in some embodiments, the simulation platform includes a simulation engine configured to create the digital content for the simulated surgical procedure. The simulation platform, via the simulation engine, is configured to receive scan data related to one or more surgical scenarios; segment the scan data; apply anatomical landmarks; apply one or more haptic values to the data; apply a templating algorithm to generate a 3D model of an anatomy; and parameterize the anatomy to create a simulated patient requiring a surgical procedure.

In some embodiments of the methods, the simulation platform is configured to monitor actions of one or more users to identify at least one of a user's field of view within the surgical environment and user interaction with a simulated object within the surgical environment based on physical user interaction with an associated hand-held component. The simulation platform is configured to then adjust output of digital content across at least one of the hand-held component and wearable display associated with a given user in response to the actions of one or more of the users. Further, in some embodiments, the haptic and/or visual feedback to be provided to a given user via an associated hand-held component and wearable display is computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment. Accordingly, the algorithm establishes a resultant effect of the interaction with the digital content that is not pre-determined.

### Specific Patient Surgical Simulation

Aspects of the invention disclose systems for providing a specific-patient simulated surgery. As noted previously, the system includes a simulation platform configured to communicate and exchange data with one or more handheld components and one or more wearable displays over a network. The platform includes a non-transitory computer-readable storage medium coupled to the processor and encoded with a computer program that causes the processor to receive data of a patient prior to the patient undergoing a surgical procedure; and generate and provide digital content comprising one or more simulated objects within a simulated surgical environment associated with a simulated surgical procedure that is to be undergone by the patient prior to the patient undergoing the actual surgical procedure. The digital content is presented to one or more users interacting with the simulated surgical environment via associated hand-held components and wearable displays. The simulated content is based, at least in part, on data received from the specific patient requiring the surgical procedure.

In certain embodiments, the simulation platform is configured to monitor user actions in the simulated surgical environment, particularly where the user actions impact the outcome of the simulated surgical procedure for the specific patient.

In some embodiments, the digital content includes virtual reality (VR) content and/or augmented reality (AR) content. In particular embodiments, the digital content is based, at least in part, on data selected from the group consisting of: data received via a standard protocol for the management and transmission of real medical images and related data; real behavioral telemetry data from a surgical procedure; real performance outcome data from a surgical procedure; and real decision data from a surgical procedure. For example, in specific embodiments, the standard protocol is Digital Imaging and Communications in Medicine (DICOM).

In some embodiments, the simulation platform further comprises a simulation engine configured to create the digital content for the simulated surgical procedure. In particular, the simulation platform, via the simulation engine is configured to receive scan data related to one or more surgical scenarios; segment the scan data; apply anatomical landmarks; apply one or more haptic values to the data; apply a templating algorithm to generate a 3D model of an anatomy; and parameterize the anatomy to create a simulated patient requiring a surgical procedure.

In various embodiments, the simulation platform is configured to monitor actions of one or more users to identify at least one of a user's field of view within the surgical environment and user interaction with a simulated object within the surgical environment based on physical user interaction with an associated hand-held component; and adjust output of digital content across at least one of the hand-held component and wearable display associated with a given user in response to the actions of one or more of the users. In particular, in some embodiments, the haptic and/or visual feedback to be provided to a given user via an associated hand-held component and wearable display is computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment. Accordingly, the algorithm establishes a resultant effect of the interaction with the digital content that is not pre-determined.

Further, in some embodiments, the simulation platform is configured to adapt the digital content in response to user actions in the virtual surgical environment; track actions of the user during the simulated surgical procedure; assess a performance of the user based on the tracked actions; determine a performance rating for the actions of the user; provide feedback to the user; and determine a pre-human competence for the surgical procedure.

In other aspects, the invention discloses a method for providing patient-specific simulated surgery. The method includes receiving, via a simulation platform, data of a patient prior to the patient undergoing a surgical procedure; and providing, via the simulation platform, digital content comprising one or more simulated objects within a simulated surgical environment associated with a simulated surgical procedure that is to be undergone by the patient prior to the patient undergoing the actual surgical procedure, which is presented to one or more users interacting with the simulated surgical environment via associated hand-held components and wearable displays. Notably, the simulated content is based, at least in part, on data received from the specific patient requiring the surgical procedure.

In certain embodiments, the simulation platform is configured to monitor user actions in the simulated surgical environment, for example, where the user actions impact the outcome of the simulated surgical procedure for the specific patient.

As disclosed herein, in some embodiments, the digital content includes at least one of virtual reality (VR) content and augmented reality (AR) content. For example, the digital content, in some embodiments, is based, at least in part, on data selected from the group consisting of: date received via a standard protocol for the management and transmission of real medical images and related data; real behavioral telemetry data from a surgical procedure; real performance outcome data from a surgical procedure; and real decision data from a surgical procedure. Further, the standard protocol may be, in certain embodiments, Digital Imaging and Communications in Medicine (DICOM).

In some embodiments, the simulation platform further comprises a simulation engine configured to create the digital content for the simulated surgical procedure. The simulation platform, via the simulation engine in certain embodiments, is configured to receive scan data related to one or more surgical scenarios; segment the scan data; apply anatomical landmarks; apply one or more haptic values to the data; apply a templating algorithm to generate a 3D model of an anatomy; and parameterize the anatomy to create a simulated patient requiring a surgical procedure.

In particular embodiments, the simulation platform is configured to monitor actions of one or more users to identify at least one of a user's field of view within the surgical environment and user interaction with a simulated object within the surgical environment based on physical user interaction with an associated hand-held component; and adjust output and synchronize sharing of digital content in real time across at least one of the hand-held component and wearable display associated with a given user in response to the actions of one or more of the users. Further, in some embodiments, haptic and/or visual feedback to be provided to a given user via an associated hand-held component and wearable display is computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment. Accordingly, the algorithm establishes a resultant effect of the interaction with the digital content that is not pre-determined.

In some embodiments, the simulation platform is further configured to adapt the digital content in response to user actions in the virtual surgical environment; track actions of the user during the simulated surgical procedure; assess a performance of the user based on the tracked actions; determine a performance rating for the actions of the user; provide feedback to the user; and determine a pre-human competence for the surgical procedure.

### Simulated Soft Tissue Suturing

Systems and methods of the invention provide solutions to the particularly challenging scenario of simulated soft tissue suturing. This requires a stable soft tissue simulation, high fidelity user input and robust tool interaction, all of which raise significant technological challenges. The invention solves the problem of these complex interactions with interactive, real time simulations, encompassing realistic soft tissue and thread behavior that provides for both continuous and interrupted suturing, and surgical square knot-tying.

Aspects of the invention disclose a system for providing a simulated soft tissue suturing procedure. The system comprises a simulation platform configured to communicate and exchange data with one or more hand-held components and one or more wearable displays over a network. The platform comprises a non-transitory computer-readable storage medium coupled to a processor and encoded with a computer program. The computer program causes the processor to generate and provide an interactive real-time suturing simulation that simulates multiple interactions between a soft tissue, a suture thread and one or more surgical instruments in a manner that movement and deformation of both the suture thread and the soft tissue are simulated using position based dynamics and collision detection.

Further, in some embodiments, the simulation platform is further configured to provide digital content comprising a plurality of three-dimensional (3D) simulated objects within a simulated surgical environment to be presented to one or more users interacting with the simulated surgical environment via associated hand-held components and wearable displays, wherein the plurality of 3D simulated objects comprises a simulated surgical instrument, a simulated soft tissue, a simulated suturing needle, and a simulated suture thread. Additionally, the simulation platform is configured to monitor actions of at least one user, including user interaction with at least one of the simulated surgical instrument, the simulated soft tissue, the simulated suturing needle, and the simulated thread based on physical user interaction with an associated hand-held component and define the user interactions for each. The simulation platform is configured to determine a type and a degree of haptic feedback to be provided to the user(s) via the associated hand-held component based, at least in part, on the defined interactions. The simulation platform is configured to transmit one or more signals to cause the hand-held component to provide the determined haptic feedback to the user and adjust output of digital content to be presented to the user via the wearable display to thereby mimic movement of any one of the simulated surgical instrument, the simulated soft tissue, the simulated suturing needle, and the simulated thread based, at least in part on the defined interactions.

In some embodiments, the defined interactions are selected from the group consisting of: interaction between the simulated suturing needle and the simulated soft tissue types; interaction between the simulated suturing needle and the simulated suture thread; interaction between the simulated suture thread and the simulated soft tissue; interaction between the user and the simulated suturing needle; interaction between the user and the simulated soft tissue; interaction between the simulated suture thread with itself; interaction between the simulated suture thread and the simulated surgical instrument; interaction between the simulated suturing needle and the simulated surgical instrument; and interaction between the simulated surgical instrument and the simulated soft tissue.

In particular embodiments, the simulation platform further includes a simulation engine configured to model the interactions using calculations associated with one or more algorithms comprising at least one of a position-based solver, a collision detection algorithm, a deformable mesh algorithm, and an adaptive mesh algorithm. For example, in some embodiments, the platform is configured to manipulate one or more of a simulated suture thread mesh, a simulated soft tissue mesh, a simulated suture thread runtime, and/or a simulated soft tissue runtime to model the movement of the simulated suture thread as it is pulled through the simulated soft tissue types. Further, in some embodiments, the movement and deformation of the simulated suture thread and the simulated soft tissue types are simulated using Position Based Dynamics to model the simulated soft tissue types and the simulated suture thread as a collection of particles controlled by a set of constraints.

The interactions, in some embodiments, one or more of prodding, grabbing, piercing, picking up, stretching, cutting, and pulling. The simulated thread and simulated suturing needle are manipulated with simulated surgical instruments controlled by the user.

In some embodiments, the interaction with the simulated suture thread and the simulated soft tissue types enables the simulation of a closure of a defect in the simulated soft tissue types and/or a joining together of separate simulated soft tissue types using one or more of a simulated continuous suturing and simulated interrupted suturing.

Further, in some embodiments, the simulation platform is configured to allow the user to choose one or more of a type of simulated suturing procedure, a type of simulated suture, a placement of the simulated suture, a direction of simulated suturing, a dominant hand for simulated suturing, and a type and/or number of simulated surgical instruments to use for the simulated soft-tissue suturing.

In certain embodiments, the haptic and/or visual feedback to be provided to a given user via an associated hand-held component and wearable display is computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined.

In another aspect, the invention discloses a method for providing a simulated soft tissue suturing procedure. The method includes generating and providing, via a simulation platform, digital content comprising a plurality of three-dimensional (3D) simulated objects. The 3D simulated objects include a simulated surgical instrument, a simulated soft tissue, a simulated suturing needle, and a simulated suture thread within a simulated surgical environment associated with an interactive real-time suturing simulation which is presented to one or more users interacting with the simulated surgical environment via an associated hand-held component and a wearable display. The interactive real-time suturing simulation simulates multiple interactions between a soft tissue, a suture thread and one or more surgical instruments in a manner that movement and deformation of both the suture thread and the soft tissue are simulated using position based dynamics and collision detection.

In some embodiments, the method further includes monitoring, via the simulation platform, actions of at least one user, including user interaction with at least one of the simulated surgical instrument, the simulated soft tissue, the simulated suturing needle, and the simulated thread based on physical user interaction with an associated hand-held component and defining, via the simulation platform, user interactions of each. The method additionally includes determining, via the simulation platform, a type and a degree of haptic feedback to be provided to the at least one user via the associated hand-held component based, at least in part, on the defined interactions. The method includes transmitting, via the simulation platform, one or more signals to cause the hand-held component to provide the determined haptic feedback to the user and further adjusting, via the simulation platform, output of digital content to be presented to the user via the wearable display to thereby mimic movement of any one of the simulated surgical instrument, the simulated soft tissue, the simulated suturing needle, and the simulated thread based, at least in part on the defined interactions.

In some embodiments of the method, the defined interactions are selected from the group consisting of: interaction between simulated suturing needle and the simulated soft tissue types; interaction between the simulated suturing needle and the simulated suture thread; interaction between the simulated suture thread and the simulated soft tissue; interaction between the user and the simulated suturing needle; interaction between the user and the simulated soft tissue; interaction between the simulated suture thread with itself; interaction between the simulated suture thread and the simulated surgical instrument; interaction between the simulated suturing needle and the simulated surgical instrument; and interaction between the simulated surgical instrument and the simulated soft tissue.

In some embodiments of the method, the simulation platform further includes a simulation engine configured to model the interactions using calculations associated with one or more algorithms comprising at least one of a position-based solver, a collision detection algorithm, a deformable mesh algorithm, and an adaptive mesh algorithm. The simulation platform of the method, in some embodiments, is further configured to manipulate one or more of a simulated suture thread mesh, a simulated soft tissue mesh, a simulated suture thread runtime, and/or a simulated soft tissue runtime to model the movement of the simulated suture thread as it is pulled through the simulated soft tissue types. Specifically, in some embodiments, the movement and deformation of the simulated suture thread and the simulated soft tissue types are simulated using Position Based Dynamics to model the simulated soft tissue types and the simulated suture thread as a collection of particles controlled by a set of constraints.

Interactions of the method, in some embodiments, include one or more of prodding, grabbing, piercing, picking up, stretching, cutting, and pulling, wherein the simulated thread and simulated suturing needle are manipulated with simulated surgical instruments controlled by the user. Accordingly, in some embodiments, the interaction with the simulated suture thread and the simulated soft tissue types enables the simulation of a closure of a defect in the simulated soft tissue types and/or a joining together of separate simulated soft tissue types using one or more of a simulated continuous suturing and simulated interrupted suturing.

In some embodiments of methods of the invention, the simulation platform is configured to allow the user to choose one or more of a type of simulated suturing procedure, a type of simulated suture, a placement of the simulated suture, a direction of simulated suturing, a dominant hand for simulated suturing, and a type and/or number of simulated surgical instruments to use for the simulated soft-tissue suturing.

In specific embodiments of the methods of the invention, the haptic and/or visual feedback to be provided to a given user via an associated hand-held component and wearable display is computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined.

### Simulated Surgical Knot-Tying

The invention provides for an interactive, real-time simulation encompassing realistic surgical square knot-tying. The movement and deformation of both the thread and the tissue may be, for example, simulated using Position Based Dynamics, which models both elements as a collection of particles controlled by a set of various constraints. The surgical instruments are controlled by the simulation platform of the system which provides an abstracted interface between the system and the hardware.

Aspects of the invention disclose a system for providing a simulated knot-tying procedure. The system includes a simulation platform configured to communicate and exchange data with one or more hand-held components and one or more wearable displays over a network. The simulation platform includes a non-transitory computer-readable storage medium coupled to the processor and encoded with a computer program. The computer program causes the processor to generate and provide an interactive real-time knot-tying simulation that simulates multiple interactions between a soft tissue, a suture thread and one or more surgical instruments in a manner that movement and deformation of both the suture thread and the soft tissue are simulated using position based dynamics and collision detection. A component of the simulation includes a model of an interaction between the suture thread and itself.

In certain embodiments, the simulation platform is further configured to provide digital content comprising a plurality of three-dimensional (3D) simulated objects within a simulated surgical environment to be presented to one or more users interacting with the simulated surgical environment via associated hand-held components and wearable displays. The plurality of 3D simulated objects includes a simulated surgical instrument, a simulated soft tissue, a simulated suturing needle, and a simulated suture thread, wherein the digital content comprises a choice of simulated knot-tying techniques. The simulation platform is configured to monitor actions of at least one user, including user interaction with at least one of the simulated surgical instrument, the simulated soft tissue, the simulated suturing needle, and the simulated thread based on physical user interaction with an associated hand-held component and define user interactions of each. Further the platform is configured to determine a type and a degree of haptic feedback to be provided to the at least one user via the associated hand-held component based, at least in part, on the defined interactions. Additionally, the simulation platform is configured to transmit one or more signals to cause the hand-held component to provide the determined haptic feedback to the user and further adjust output of digital content to be presented to the user via the wearable display to thereby mimic movement of any one of the simulated surgical instrument, the simulated soft tissue, the simulated suturing needle, and the simulated thread based, at least in part, on the defined interactions.

In further embodiments, the defined interactions are selected from the group consisting of: interaction between simulated suturing needle and the simulated soft tissue types; interaction between the simulated suturing needle and the simulated suture thread; interaction between the simulated suture thread and the simulated soft tissue; interaction between the user and the simulated suturing needle; interaction between the user and the simulated soft tissue; interaction between the simulated suture thread with itself; interaction between the simulated suture thread and the simulated surgical instrument; interaction between the simulated suturing needle and the simulated surgical instrument; and interaction between the simulated surgical instrument and the simulated soft tissue.

In certain embodiments, the simulation platform further comprises a simulation engine configured to model the interactions using calculations associated with one or more algorithms comprising at least one of a position-based solver, a collision detection algorithm, a deformable mesh algorithm, and an adaptive mesh algorithm. For example, in some embodiments, the simulation platform is configured to manipulate one or more of a simulated suture thread mesh, a simulated soft tissue mesh, a simulated suture thread runtime, and/or a simulated soft tissue runtime to model the movement of the simulated suture thread as it is pulled through the simulated soft tissue types. In some embodiments, the movement and deformation of the simulated suture thread and the simulated soft tissue types are simulated using Position Based Dynamics to model the simulated soft tissue types and the simulated suture thread as a collection of particles controlled by a set of constraints. Further, in some embodiments of the methods, the interactions include one or more of prodding, grabbing, piercing, picking up, stretching, cutting, and pulling, wherein the simulated thread and simulated suturing needle are manipulated with one or more simulated surgical instruments controlled by the user.

In particular embodiments of systems of the invention, the simulated knot-tying procedure includes wrapping the simulated suture thread around a simulated surgical instrument, and sliding the wrapped simulated suture thread off of the simulated surgical instrument such that the simulated suture thread retains its wrapped shape once it is disengaged from the simulated surgical instrument.

In some embodiments, the simulation platform is configured to detect when a user is attempting to tie a simulated surgical knot, when the simulated surgical knot has been completed, and/or when the user has not completed a simulated surgical knot. For example, in some embodiments, the simulation platform is configured to simulate an unraveling of the simulated surgical knot when the user has not completed a simulated surgical knot.

In some embodiments, the haptic and/or visual feedback to be provided to a given user via an associated hand-held component and wearable display is computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined.

In another aspect, the invention discloses a method for providing a simulated knot-tying procedure. The method includes generating and providing, via a simulation platform, digital content comprising a plurality of three-dimensional (3D) simulated objects comprising a simulated surgical instrument, a simulated soft tissue, a simulated suturing needle, and a simulated suture thread within a simulated surgical environment associated with an interactive real-time knot-tying simulation. The simulation is presented to one or more users interacting with the simulated surgical environment via associated hand-held components and wearable displays The interactive real-time knot-tying simulation simulates multiple interactions between a soft tissue, a suture thread and one or more surgical instruments in a manner that movement and deformation of both the suture thread and the soft tissue are simulated using position based dynamics and collision detection and wherein a component of the simulation includes a model of an interaction between the suture thread and itself.

In some embodiments, the method further includes monitoring, via the simulation platform, actions of at least one user, including user interaction with at least one of the simulated surgical instrument, the simulated soft tissue, the simulated suturing needle, and the simulated thread based on physical user interaction with an associated hand-held component and defining user interactions for each. The method includes determining, via the simulation platform, a type and a degree of haptic feedback to be provided to the at least one user via the associated hand-held component based, at least in part, on the defined interactions. Additionally, the method includes transmitting, via the simulation platform, one or more signals to cause the hand-held component to provide the determined haptic feedback to the user and further adjusting, via the simulation platform, output of digital content to be presented to the user via the wearable display to thereby mimic movement of any one of the simulated surgical instrument, the simulated soft tissue, the simulated suturing needle, and the simulated thread based, at least in part, on the defined interactions.

In particular embodiments of the method, the defined interactions are selected from the group consisting of: interaction between simulated suturing needle and the simulated soft tissue types; interaction between the simulated suturing needle and the simulated suture thread; interaction between the simulated suture thread and the simulated soft tissue; interaction between the user and the simulated suturing needle; interaction between the user and the simulated soft tissue; interaction between the simulated suture thread with itself; interaction between the simulated suture thread and the simulated surgical instrument; interaction between the simulated suturing needle and the simulated surgical instrument; and interaction between the simulated surgical instrument and the simulated soft tissue.

Further, in some embodiments, the simulation platform further comprises a simulation engine configured to model the interactions using calculations associated with one or more algorithms comprising at least one of a position-based solver, a collision detection algorithm, a deformable mesh algorithm, and an adaptive mesh algorithm. For example, in some embodiments, the simulation platform is configured to manipulate one or more of a simulated suture thread mesh, a simulated soft tissue mesh, a simulated suture thread runtime, and/or a simulated soft tissue runtime to model the movement of the simulated suture thread as it is pulled through the simulated soft tissue types. In particular embodiments, the movement and deformation of the simulated suture thread and the simulated soft tissue types are simulated using position based dynamics to model the simulated soft tissue types and the simulated suture thread as a collection of particles controlled by a set of constraints.

In certain embodiments of the method, the interactions include one or more of prodding, grabbing, piercing, picking up, stretching, cutting, and pulling, wherein the simulated thread and simulated suturing needle are manipulated with one or more simulated surgical instruments controlled by the user.

In some embodiments of the method, the simulated knot-tying procedure includes wrapping the simulated suture thread around a simulated surgical instrument, and sliding the wrapped simulated suture thread off of the simulated surgical instrument such that the simulated suture thread retains its wrapped shape once it is disengaged from the simulated surgical instrument.

Further, in some embodiments, the simulation platform is configured to detect when a user is attempting to tie a simulated surgical knot, when the simulated surgical knot has been completed, and/or when the user has not completed a simulated surgical knot. For example, in some embodiments, the simulation platform is configured to simulate an unraveling of the simulated surgical knot when the user has not completed a simulated surgical knot.

In particular embodiments, the haptic and/or visual feedback to be provided to a given user via an associated hand-held component and wearable display is computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined.

### Digital Twins

The invention discloses systems and methods for providing a digital twin of, for example, a medical device, that may be used for research and validation of the device during design and development. Digital twins of the systems of the invention provide a virtual model designed to accurately reflect the physical object.

In aspects, the invention discloses a system for providing a medical device simulation. The system includes a simulation platform configured to communicate and exchange date with one or more hand-held components and one or more wearable displays over a network. The platform includes a non-transitory computer-readable storage medium coupled to the processor and encoded with a computer program. The computer program is operable to cause the platform to generate and provide digital content comprising at least a simulated medical device in a simulated surgical environment to be presented to one or more users interacting with the simulated surgical environment via associated hand-held components and wearable displays. Further, the simulated medical device is a digital twin of a real-world medical device such that the platform is configured to provide a user with highly accurate visualizations of and interaction with the simulated medical device in the simulated surgical environment that match visualizations of and interaction with the real-world medical device in a real-world surgical environment.

In some embodiments, the simulation platform is configured to monitor user actions within the simulated surgical environment. In particular, the user interactions include at least user interaction with the simulated medical device based on physical user interaction with an associated hand-held component. Further, in some embodiments, the simulation platform is configured to adjust output of digital content to thereby mimic operation and/or movement of the simulated objected.

In some embodiments, the simulation platform is configured to collect data associated with the user's interaction with and operation of the simulated medical device within the simulated surgical environment. In particular, in some embodiments the data collected is used to validate design and/or performance of the real-world medical device. For example, the data may provide a basis for a redesign of the real-world medical device. In some embodiments, the data provides a risk assessment associated with the operation of the real-world medical device.

In various embodiments, the simulation platform is configured to adapt the digital content in response to the one or more user actions in the virtual surgical environment. For example, in some embodiments, the simulation platform is further configured to track actions of the one or more users; assess a performance of the one or more users based on the tracked actions; determine a performance rating for the actions of the user; and provide feedback to the one or more users associated with their performance rating.

In some embodiments, the haptic and/or visual feedback to be provided to a given user via an associated hand-held component and wearable display is computed using an algorithm based on one or more input parameters associated with the hand-held component and the digital twin and a visual effect on the digital twin within the simulated surgical environment, wherein the algorithm establishes a resultant effect of the interaction with the digital content that is not pre-determined.

Aspects of the invention disclose a method for providing a medical device simulation. The method includes generating and providing, via a simulation platform, digital content comprising at least a simulated medical device in a simulated surgical environment to be presented to one or more users interacting with the simulated surgical environment via associated hand-held components and wearable displays. The simulated medical device is a digital twin of a real-world medical device such that the platform is configured to provide a user with highly accurate visualizations of and interaction with the simulated medical device in the simulated surgical environment that match visualizations of and interaction with the real-world medical device in a real-world surgical environment.

In some embodiments of the method, the simulation platform is configured to monitor user actions within the simulated surgical environment. The user actions, in particular embodiments may be at least user interaction with the simulated medical device based on physical user interaction with an associated hand-held component. The simulation platform, in example embodiments, is configured to adjust output of digital content to thereby mimic operation and/or movement of the simulated objected.

In particular embodiments of the method, the simulation platform is configured to collect data associated with the user's interaction with and operation of the simulated medical device within the simulated surgical environment. For example, in some embodiments, the data collected is used to validate design and/or performance of the real-world medical device. Further, in some embodiments, the data provides a basis for a redesign of the real-world medical device. In specific embodiments, the data provides a risk assessment associated with the operation of the real-world medical device.

The simulation platform is configured, in some embodiments, to adapt the digital content in response to the one or more user actions in the virtual surgical environment. Further, in particular embodiments, the simulation platform is configured to track actions of the one or more users; assess a performance of the one or more users based on the tracked actions; determine a performance rating for the actions of the user; and provide feedback to the one or more users associated with their performance rating.

In some embodiments, the haptic and/or visual feedback to be provided to a given user via an associated hand-held component and wearable display is computed using an algorithm based on one or more input parameters associated with the hand-held component and the digital twin and a visual effect on the digital twin within the simulated surgical environment, wherein the algorithm establishes a resultant effect of the interaction with the digital content that is not pre-determined.

### Virtual Imaging in the Simulated Surgical Environment

The invention provides novel systems and methods for simulated medical imaging within a simulated surgical environment.

In one aspect, the invention discloses a system for providing simulated medical imaging within a simulated medical environment. The system includes a simulation platform configured to communicate and exchange data with one or more hand-held components and one or more wearable displays over a network. The platform includes a non-transitory computer-readable storage medium coupled to the processor and encoded with a computer program. The computer program is operable to cause the platform to generate and provide a simulated medical environment that comprises a simulated anatomical structure and a simulated medical imaging device, wherein the simulated medical environment is configured for a user to interact with the simulated medical imaging device to virtually image the simulated anatomical structure and generate a simulated medical image of the simulated anatomical structure from the simulated medical imaging device.

In some embodiments, the simulation platform is further configured to generate and provide digital content comprising at least a simulated medical imaging device and a simulated anatomical structure in a simulated medical environment. The digital content is presented to one or more users interacting with the simulated medical environment via associated hand-held components and wearable displays. Further, the simulation platform is configured to monitor user actions within the simulated medical environment, wherein said user interactions comprise at least user interaction with the simulated medical imaging device relative to the simulated anatomical structure based on physical user interaction with an associated hand-held component. The simulation platform is configured to generate, in response to user interaction with the simulated medical imaging device, a simulated medical image of the simulated anatomical structure obtained from the simulated imaging device.

In some embodiments of the systems, the simulated medical image includes one or more of a dynamic depiction of the simulated anatomical structure, a static depiction of the simulated anatomical structure, a dynamic depiction of fluid flow within the anatomical structure, and a simulated medical instrument in the simulated medical environment.

In some embodiments, generating the simulated medical image comprises generating a two-dimensional (2D) slice of the simulated anatomical structure based on a position of the simulated imaging device relative to the simulated anatomical structure. For example, in some embodiments, generating the 2D slice of the simulated anatomical structure includes applying one or more anatomical landmarks to the simulated anatomical structure; applying one or more haptic values to the simulated anatomical structure; and applying a templating algorithm, wherein the templating algorithm recognizes and tags the simulated anatomical structure in a two-dimensional view to parameterize the simulated anatomical structure to generate the simulated medical image as a view represented by the simulated medical imaging device. Further, the anatomical landmarks may appear on an output of the generated 2D slice, such that the anatomical landmarks appear differently according to a type of tissue associated with the simulated medical image and/or a non-biological component present in the simulated medical image.

In some embodiments of the systems of the invention, generating the 2D slice of the simulated anatomical structure further includes performing calculations at a shader level of the simulated medical image; and adding one or more effects on to the calculations to generate the simulated medical image.

In some embodiments, the simulation platform is configured to generate a simulated dynamic image of a complex simulated medical instrument moving through a simulated patient's internal anatomy. The simulation platform may be configured to use a spline to apply deformations to a three-dimensional (3D) simulated medical instrument such that the simulated medical instrument travels along a curved path in 3D space; calculate and adjust vertices of a mesh associated with the simulated medical instrument based on the vertices' original positions relative to the spline used to apply the deformations, such that the calculations are performed each time the position of the mesh is adjusted during operation of the simulated medical instrument; and selectively lock portions of the mesh to allow the selected portions to deform as a group.

The simulation platform is configured, in some embodiments, to generate a simulated ultrasound image by generating a two-dimensional (2D) slice based on a defined position of all simulated tissues marked to appear on the simulated ultrasound image, concatenating a plurality of images corresponding to one or more types of simulated tissues and simulated surgical instruments, and applying one or more layers of a post processing.

The simulation platform is configured, in some embodiments, to run a collision detection and resolution algorithm, wherein the algorithm calculates a current position and an orientation of the user's hand within the simulated medical environment to determine an optimal position of a simulated imaging device probe.

In other aspects, the invention discloses methods for providing simulated medical imaging within a simulated medical environment. The method includes generating and providing, via a simulation platform, digital content comprising a simulated medical environment that comprises a simulated anatomical structure and a simulated medical imaging device, wherein the simulated medical environment is configured for a user to interact with the simulated medical imaging device to virtually image the simulated anatomical structure via associated hand-held components and wearable displays and to generate a simulated medical image of the simulated anatomical structure from the simulated medical imaging device.

In some embodiments, the method further includes monitoring, via the simulation platform, user actions within the simulated medical environment, wherein said user interactions comprise at least user interaction with the simulated medical imaging device relative to the simulated anatomical structure based on physical user interaction with an associated hand-held component; and generating, via the simulation platform and in response to user interaction with the simulated medical imaging device, a simulated medical image of the simulated anatomical structure obtained from the simulated imaging device. Further, the simulated medical image may include one or more of a dynamic depiction of the simulated anatomical structure, a static depiction of the simulated anatomical structure, a dynamic depiction of fluid flow within the anatomical structure, and a simulated medical instrument in the simulated medical environment.

Generating the simulated medical image may include, in some embodiments, generating a two-dimensional (2D) slice of the simulated anatomical structure based on a position of the simulated imaging device relative to the simulated anatomical structure. For example, in some embodiments generating the 2D slice of the simulated anatomical structure includes applying one or more anatomical landmarks to the simulated anatomical structure; applying one or more haptic values to the simulated anatomical structure; applying a templating algorithm, wherein the templating algorithm recognizes and tags the simulated anatomical structure in a two-dimensional view to parameterize the simulated anatomical structure to generate the simulated medical image as a view represented by the simulated medical imaging device. The anatomical landmarks may appear on an output of the generated 2D slice, such that the anatomical landmarks appear differently according to a type of tissue associated with the simulated medical image and/or a non-biological component present in the simulated medical image.

In some embodiments, generating the 2D slice of the simulated anatomical structure further includes performing calculations at a shader level of the simulated medical image; and adding one or more effects on to the calculations to generate the simulated medical image.

In some embodiments of methods of the invention, simulation platform is configured to generate a simulated dynamic image of a complex simulated medical instrument moving through a simulated patient's internal anatomy. In particular embodiments, the simulation platform is configured to use a spline to apply deformations to a three-dimensional (3D) simulated medical instrument such that the simulated medical instrument travels along a curved path in 3D space;

calculate and adjust vertices of a mesh associated with the simulated medical instrument based on the vertices' original positions relative to the spline used to apply the deformations, such that the calculations are performed each time the position of the mesh is adjusted during operation of the simulated medical instrument; and selectively lock portions of the mesh to allow the selected portions to deform as a group.

Further, in some embodiments of the methods, the simulation platform is configured to generate a simulated ultrasound image by generating a 2D slice based on a defined position of all simulated tissues marked to appear on the simulated ultrasound image, concatenating a plurality of images corresponding to one or more types of simulated tissues and simulated surgical instruments, and applying one or more layers of a post processing.

In particular embodiments, the simulation platform is configured to run a collision detection and resolution algorithm, wherein the algorithm calculates a current position and an orientation of the user's hand within the simulated medical environment to determine an optimal position of a simulated imaging device probe.

Simulated Software within the Simulated Surgical Environment

The invention provides systems and methods for interacting with virtual software in the virtual medical environment, for example, software that is used to operate a medical device or machine.

In one aspect, the invention discloses a system for providing simulated software and interaction therewith within a simulated medical environment. The system includes a simulation platform a simulation platform configured to communicate and exchange data with one or more hand-held components and one or more wearable displays over a network, wherein the platform comprises a non-transitory computer-readable storage medium coupled to the processor and encoded with a computer program. The computer program is operable to cause the platform to generate and provide a simulated medical environment that comprises a simulated device running simulated medical software thereon that is reflective of a real-world version of the medical software. Further, the simulated medical environment is configured for a user to interact with a simulated user interface associated with the simulated medical software running on the simulated device via one or more hand-held components and a wearable display.

In some embodiments, the user interaction with the simulated user interface allows for user operation of and interaction with the simulated device and/or user operation of and interaction with one more other simulated devices within the simulated medical environment. For example, in some embodiments, the simulated device and the one or more other simulated devices are selected from the group consisting of a computing device and a medical device. The computing device is selected from the group consisting of a personal computer (PC), a tablet, and a smartphone, in some embodiments. The medical device may be selected from the group consisting of a medical imaging machine, an anesthesia machine, an EKG/ECG machine, a surgical treatment machine, a patient monitoring machine, a hemodialysis machine, and one or more operating room monitoring devices.

In some embodiments, the simulated medical software and the simulated user interface associated with the simulated device are digital twins of corresponding real-world version of the medical software and a real-world user interface associated with a real-world device, respectively, such that the platform is configured to provide a user with highly accurate visualizations of and interaction with the associated simulated user interface in the simulated medical environment that match visualizations of and interaction with a real-world user interface of a real-world device in a real-world medical environment.

In particular embodiments, the platform is configured to collect data associated with user interaction with the simulated user interface associated with the simulated medical software to operate the simulated device within the simulated medical environment. The data collected may be used to validate the design and/or performance of the real-world version of the medical software.

In some embodiments, the simulation platform is further configured to generate and provide digital content comprising at least a simulated device running simulated medical software thereon and a simulated user interface associated with the simulated medical software in a simulated medical environment. Further the simulation platform is configured to monitor user actions within the simulated medical environment. The user interactions include at least user interactions with the simulated user interface based on physical user interaction with one or more associated hand-held components. The simulation platform may be further configured to generate, in response to user interaction with the simulated user interface, user input with the simulated user interface and operation of the simulated device in response thereto.

In some embodiments, the simulated medical software reflective of a real-world version of the medical software is selected from the group consisting of a medical imaging software, a medical drug delivery software, a medical diagnostic software, a medical therapy software, and a patient monitoring software.

In another aspect, the invention discloses a method for providing simulated software and interaction therewith within a simulated medical environment. The method includes generating and providing, via a simulation platform, a simulated medical environment that comprises a simulated device running simulated medical software thereon that is reflective of a real-world version of the medical software, wherein the simulated medical environment is configured for a user to interact with a simulated user interface associated with the simulated medical software running on the simulated device via one or more hand-held components and a wearable display.

In some embodiments of the method, the user interaction with the simulated user interface allows for user operation of and interaction with the simulated device and/or user operation of and interaction with one more other simulated devices within the simulated medical environment. The simulated device and the one or more other simulated devices are selected from the group consisting of a computing device and a medical device, in some embodiments. The computing device may be selected from the group consisting of a personal computer (PC), a tablet, and a smartphone. The medical device may be selected from the group consisting of a medical imaging machine, an anesthesia machine, an EKG/ECG machine, a surgical treatment machine, a patient monitoring machine, a hemodialysis machine, and one or more operating room monitoring devices.

In some embodiments of the methods, the simulated medical software and the simulated user interface associated with the simulated device are digital twins of a corresponding real-world version of the medical software and a corresponding real-world user interface associated with a real-world device, respectively, such that the platform is configured to provide a user with highly accurate visualizations of and interaction with the associated simulated user interface in the simulated medical environment that match visualizations of and interaction with a real-world user interface of a real-world device in a real-world medical environment.

In some embodiments of the methods, the simulation platform is configured to collect data associated with user interaction with the simulated user interface associated with the simulated medical software to operate the simulated device within the simulated medical environment. For example, the data collected may be used to validate the design and/or performance of the real-world version of the medical software.

In some embodiments, the method further includes generating and providing, via the simulation platform, digital content comprising at least a simulated device running simulated medical software thereon and a simulated user interface associated with the simulated medical software in a simulated medical environment; monitoring, via the simulation platform, user actions within the simulated medical environment, wherein said user interactions comprise at least user interactions with the simulated user interface based on physical user interaction with one or more associated hand-held components; and generating, via the simulation platform and in response to user interaction with the simulated user interface, user input with the simulated user interface and operation of the simulated device in response thereto.

In some embodiments of the methods, the simulated medical software reflective of a real-world version of the medical software is selected from the group consisting of a medical imaging software, a medical drug delivery software, a medical diagnostic software, a medical therapy software, and a patient monitoring software.

### BRIEF DESCRIPTION OF THE DRAWINGS

FIG. 1 is a block diagram of systems of the invention, according to one embodiment, comprising modules illustrating the single and multi-user capabilities for simulating a surgical environment.

FIG. 2 is a block diagram illustrating one embodiment of an exemplary system consistent with the present disclosure.

FIG. 3 is a block diagram of one embodiment of a haptics intelligence engine of systems of the invention.

FIG. 4 illustrates an exemplary technique of systems of the invention for enhancing the surface detail of a haptic object.

FIG. 5 illustrates one embodiment of a texture atlas according to one embodiment of systems of the invention.

FIG. 6 illustrates an exemplary technique of systems of the invention for rendering the mesh wherein the tissue type data in the voxel representation is used to dynamically generate UV coordinates that can be stored in the mesh data.

FIG. 7 shows a user interacting with digital content according to one embodiment of systems 100 of the invention.

FIG. 8 shows digital content of a representation of one embodiment of the simulated surgical environment of the invention in which multiple users are engaged within the environment.

FIG. 9A and FIG. 9B show digital content according to one embodiment of systems of the invention of haptic gates and haptic guides applied in a surgical simulation.

FIG. 10 illustrates one embodiment of methods of the invention for providing a simulated surgical environment.

FIG. 11 shows digital content of a virtual patient in the simulated surgical environment according to one embodiment of systems and methods of the invention.

FIG. 12 shows digital content of a knee according to one embodiment of systems of the invention developed from specific-patient scan data imported into one embodiment of systems of the invention.

FIG. 13 illustrates one embodiment of a method of the invention for providing a patient-specific surgical environment.

FIG. 14 illustrates one embodiment of a method of the invention for providing patient-specific simulated surgery according to systems and methods of the invention.

FIG. 15 shows digital content of simulated soft tissue suturing according to one embodiment of the invention, in which the user has grabbed the tissue using tissue forceps to stretch it.

FIG. 16 shows digital content illustrating the user piercing the particular soft tissue with an instrument such as a curved needle according to one embodiment of the invention.

FIG. 17 shows digital content according to one embodiment of the invention in which simulated sutures are made in the simulated soft tissue.

FIG. 18 shows digital content, according to one embodiment, of thread being tightened in the simulated surgical suturing of the invention.

FIG. 19 illustrates a method for providing a simulated soft tissue suturing procedure according to one embodiment of the invention.

FIG. 20 shows digital content simulating soft tissue suturing according to one embodiment of the invention.

FIG. 21 shows one layer for simulating wrapping suture thread around a surgical instrument in preparation of making a square knot according to one embodiment of the invention.

FIG. 22 shows the underlying construct for the stable completion of simulated knot-tying according to one embodiment of the invention with the suture material being held under pressure as the knot is tightened.

FIG. 23 illustrates a method for providing a simulated knot-tying procedure according to one embodiment of the invention.

FIG. 24 shows a robotic medical device interacting with digital content according to one embodiment of the invention.

FIG. 25 illustrates one embodiment of a method for providing a medical device simulation.

FIG. 26 illustrates generation an imaging slice of a patient from an arbitrary position controlled by the user according to one embodiment of the invention.

FIG. 27 shows digital content of a simulated ultrasound image generated within the simulated surgical environment according to one embodiment of the systems and methods of the invention.

FIG. 28 shows digital content of a simulated probe device and imaging procedure within the simulated surgical environment, with the simulated image displayed according to one embodiment of the invention.

FIG. 29A shows digital content of the virtual surgical environment including the virtual image to simulate a section of suprapatellar nailing of tibial fractures from a simulated surgical procedure.

FIG. 29B is a cutout of the digital content of the simulated image in the simulated surgical environment showing a section of suprapatellar nailing of tibial fractures from the simulated surgical procedure.

FIG. 30 illustrates a method for providing simulated medical imaging according to one embodiment of the invention.

FIG. 31 shows digital content according to one embodiment of the systems of the invention in which users interact with digital images within the simulated surgical environment.

FIG. 32, FIG. 33, and FIG. 34 show digital content provided according to one embodiment of systems and methods of the invention.

FIG. 35 illustrates one embodiment of a method for providing simulated software and interaction therewith within a simulated medical environment.

### DETAILED DESCRIPTION

The present invention recognizes the drawbacks of current VR, AR, MR, and cross-reality (XR) surgical simulation systems, namely the technical challenges that limit the use of these systems in an immersive, multi-user, collaborative, simulated surgical environment. Systems of the invention are built into a platform that includes a suite of features providing scalable, cost-effective, and realistic simulations that may be integrated into a surgical training curriculum. In particular, the invention provides for multi-user interaction within a hardware/software agnostic platform. The systems and methods of the invention address technical issues related to the realism and accuracy necessary for effective use of simulated surgical procedures as a training tool. As such, systems and methods of the invention provide for dynamic, frictionless remote collaboration, unlimited repetition, and assessment data insight on capability and risk, which reduces costs and improves patient outcomes.

As described in detail herein, the invention accomplishes realistic, real-time, free-form simulation of surgical procedures by combining the three simulation disciplines-physical, visual, and haptic-in novel ways in a system encompassing various engines capable of a new level of immersion. The system is built on improved techniques such as voxel representation, position-based dynamics, and solid constructive geometry to generate simulations that exhibit emergent properties and give infinite variability in outcome. As a result, the simulation experience is fluid and natural and in the direct control of the user. Accordingly, the platform allows for users to decide how to interact with the tissues of the virtual patient, such as choosing where to cut, to resect or suture, and seeing and feeling the direct impact of these decisions. This is accomplished in a generalized way without the need for custom hardware.

Because surgeons mainly depend on perceptual-motor coordination and contact to carry out procedures, the invention provides essential haptic as well as visual and audio cues that closely resemble those found in real situations. Particularly, the systems of the invention combine a mix of haptic and non-haptic VR, AR, MR, and/or XR experiences at scale in a platform that provides remote collaboration that may be integrated into the live operating room. The system is built into a unique infrastructure combining visual, audible and haptic capabilities for teaching and assessing surgical skills, with particular focus on precision skills transfer. Accordingly, the platform may be an education platform for use in surgical/medical training curricula.

As described in detail herein, the invention discloses systems and methods for accurate tissue property acquisition and deformation models that can accommodate large non-linear deformations such as is found in real surgical procedures. Further, the system records and amasses data used to generate models and which may be used for customized surgical simulations, for example, patient-specific and/or specific patient surgical simulations. Accordingly, the invention provides systems for simulating the required precise anatomy and tissue deformation in order to plan for, or determine the outcome of, a surgical procedure.

Systems of the invention also provide for real-time simulation encompassing realistic soft tissue interactions so that more complex behaviors such as bleeding and contractions, soft tissue suturing, and knot-tying may be simulated. As described in detail below, the invention addresses the technical challenges of simulating soft tissue suturing by providing a stable simulation, high fidelity user input, high fidelity tissue dynamics, and robust tool interaction. Systems of the invention provide for realistic rendering, i.e. realistic image synthesis, especially those focusing on non-rigid organs, using hardware accelerated rendering, and haptic feedback to achieve intricate tissue/tool interactions for complex skills training.

As disclosed in more detail below, systems of the invention also provide for medical device simulation, for example digital twins that may be used for device development and/or validation. Further, systems of the invention are capable of simulated medical imaging techniques including probe control and fluoroscopy techniques, as well as providing simulated software in the simulated surgical environment, for example to control a simulated medical device.

As used herein, competence in the field of medicine is the collection of knowledge, abilities, skills, experiences, and behaviors—for example those falling under the categories of science competencies, procedural competencies, thinking and reasoning competencies, and intra/interpersonal competencies—which leads to effective performance in the medical field. Accordingly, the robust combination of features and modules provided by systems of the invention may be used to achieve competence in the simulated surgical and/or medical environment as applied to a virtual patient. This realistic simulated surgical environment experience, with rehearsal and assessment in a team environment, results in pre-human competence, wherein competence is achieved in the simulated surgical environment to enable transfer of these skills to a real-world medical procedure and patient.

### System/Platform

Systems and methods of the invention are integrated into a unique infrastructure, i.e. platform, that combines visual, audible and haptic capabilities for teaching and assessing surgical skills, with particular focus on precision skills transfer.

In general, realistic VR/AR/MR/XR experiences require processing large amounts of data quickly. The systems, software and methods described herein are configured to create an accurate training experience by providing a simulated surgical environment that includes a realistic level of visual, and/or auditory, and/or tactile data (detail) that is presented without processing delay such that the interaction is seamless to the user. Haptic details are enhanced using a unique algorithmic process that provides a simulated, mixed, or virtual environment with dynamic haptic feedback that does not require pre-programmed interactions for each tool/target/object combination, resulting in true free-form engagement within the simulated surgical environment.

As an overview, in non-limiting examples, the architecture of systems of the invention include a simulation platform, which includes a haptic intelligence engine and a simulation engine. The simulation platform may include 3D anatomy generated by artists/designers, or by Digital Imaging and Communications in Medicine (DICOM) scan data. As described in more detail below, the haptic intelligence engine is capable of updating haptics faster than visual data update, such that calculations are performed via compute shaders, unique collision algorithms, and a system for proprioceptive decoupling and alignment.

The visual and haptic algorithmic rendering and audio integration represent unconventional data processing/manipulation steps that achieve improved realism in simulated surgical experiences. Further, the platform provides a dynamic surgical simulation experience wherein the platform is capable of adjusting the progression and outcome of surgical procedures and the surgical environment based on data received from user interaction within the surgical environment. Accordingly, systems of the invention provide for dynamic and variable surgical simulation experiences that are not limited to tasks performed in a prescribed order. This improved realism allows for the real-time simulation of highly complex behaviors for both anatomical structures and surgical tools, and can be applied to complex surgical procedures for a fully realistic computer simulated surgery.

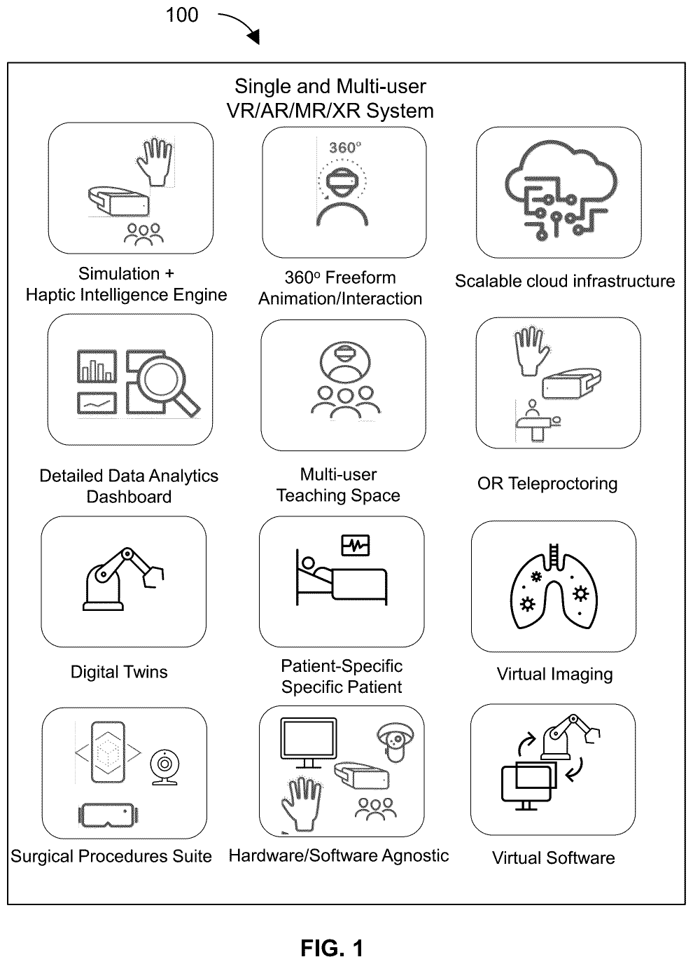

FIG. 1 is a block diagram of a system 100 of the invention, according to one embodiment. FIG. 1 provides a broad illustration of features and/or modules that may be provided by the system for single and multiuser interaction within the simulated surgical environment. Systems of the invention incorporate novel technical solutions to challenges related to the creation of surgical VR-based simulators to overcome the limitations of existing computer implemented medical training technologies. The invention addresses model acquisition and simplification, tissue property acquisition, and haptic rendering. Systems of the invention are built into a unique infrastructure combining visual, audible and haptic capabilities, for example, as described in U.S. Pat. Nos. 10,698,493, 11,256,332, 11,272,988, U.S. application Ser. No. 17/592,775 and U.S. application Ser. No. 17/558,329, each incorporated by reference in their entirety herein.

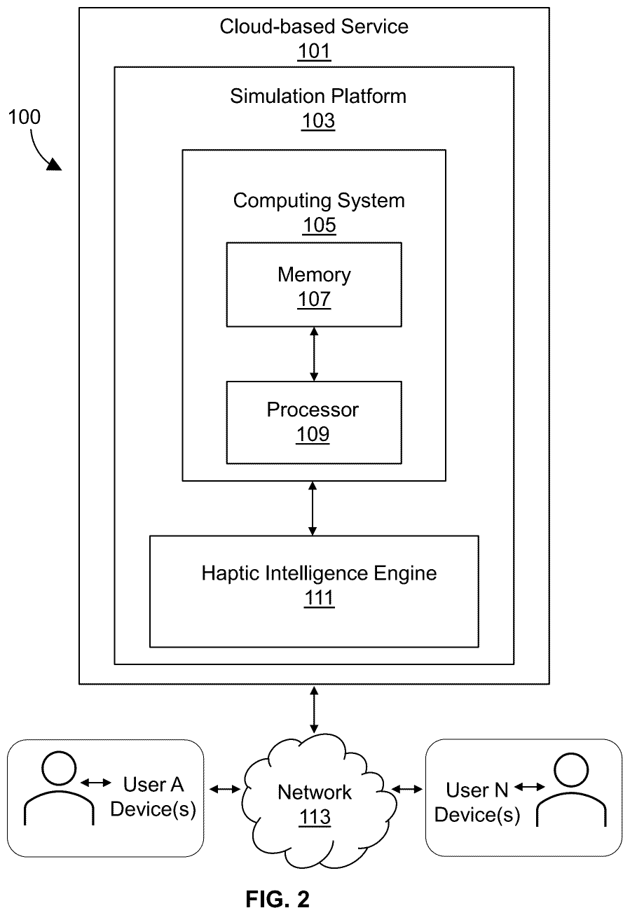

FIG. 2 is a block diagram illustrating another embodiment of an exemplary system 100 consistent with the present disclosure. As described in more detail herein, the system includes a simulation platform which may be embodied on an internet-based computing system/service. As shown, the system architecture backend may be a cloud based service. The system architecture may be multi-tenant.

Further, the systems include a haptic intelligence engine that uses an algorithmic approach to haptic tool action and programming, allowing for rapid development of tool/object haptic interaction. As used herein, a haptic tool may be any simulated item that can affect a haptic response on a secondary simulated item (haptic target), (e.g. a scalpel, finger, hammer, retractor, etc.). The haptic target may be any simulated item that can be affected by a haptic tool. A haptic action may be the interaction between a haptic tool and a haptic target, often when a force is applied.

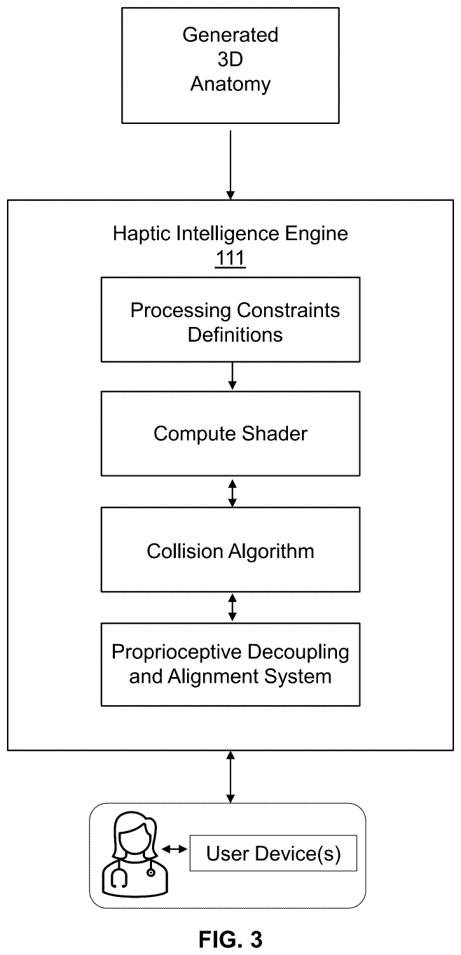

FIG. 3 is a block diagram illustrating one embodiment of a haptics intelligence engine 111 of systems of the invention. The 3D anatomy may be received by the haptic intelligence engine 111 from multiple sources. For example, the 3D anatomy may be generated by different sources such as by artists and/or designers, or DICOM scan data. The haptic intelligence engine may include features that allow for haptics to be updated faster, such as ten times faster, than visuals to provide a realistic interaction with tissue types. For example, the haptic intelligence engine may include a system for defining processing constraints/rules, a compute shader to perform calculations, a collision algorithm, and a system for proprioceptive decoupling and alignment.

Algorithmic calculations may be made to determine a haptic value based on properties of the virtual object and the virtual surgical instrument. This haptic value may be used to transmit haptic feedback to the hand-held device. Therefore, unlike contemporary systems, the systems of the invention do not specifically program the combination of each type of interaction on a case-by-case basis, and are not limited to pre-determined interactions. A virtual object may be displayed and the movement of the virtual surgical instrument may be moved based on a user's movement of a hand-held device. Moreover, the visual feedback may also be algorithmically configured to be displayed in accordance with the type of interaction.

This novel approach to achieving unique, free-form interactions of each haptic tool with haptic targets such as tissues or substances is made possible by defining a limited number of possible affordances that a haptic tool can implement along with a matching number of possible susceptibilities that a haptic target can respond to. These affordances and susceptibilities are each assigned a numerical value which are then combined to establish the nature of the interaction between the haptic tools and haptic targets.

For example, tools may have a defined sharpness affordance and targets may have a defined sharpness susceptibility, such that these values may be combined algorithmically and translated into actions and effects in the simulation. This creates an abstraction layer between a tool and its affordances, and a target and its susceptibilities, which enables the systematic and generalizable calculation of effects generated. As a result, complexity and effort are proportional to the number of affordances and susceptibilities of the tools and targets of the simulation, which allows the systems of the invention to store data related to the virtual/augmented/cross-reality experience in a highly efficient manner. As noted above, this allows the handling of interactions between tools and targets to be conducted through an abstraction layer, which radically reduces the amount of programming effort needed to support the required tools and targets needed to fulfil simulation.

As a result, systems of the invention provide for the depiction of real-time, realistic visual effects. In non-limiting examples, the resultant visual effect may be a deformation, a burning, a color change or discoloration, a clean cut, a minor cut, tearing or ripping, grinding, freezing, or any combination thereof. The visual effect may be a 2-dimensional effect, a 3-dimensional effect, or both. Additionally and/or alternatively, the visual feedback may not comprise a visual effect on the virtual object when the algorithmic calculation of the susceptibility value, the input, and the affordance value indicates the interaction has no effect on the virtual object. In some examples, the virtual object may be associated with a plurality of susceptibility values, such that each of the plurality of susceptibility values corresponds to a type of interaction. As described above, the virtual surgical instrument(s) may be associated with a plurality of affordance values, for example, each of the plurality of affordance values may correspond to a type of interaction.

Further, systems of the invention may utilize high frequency haptic surface data, a mechanism for creating high volumes of haptic information via an innovative use of 2-dimensional (2D) UV coordinates. The standard approach to defining the shape of an object in 3D simulations using haptics is use polygons as an easily computable approximation. By definition these polygons are flat and contain no surface detail of any kind. In order to increase the level of detail of the haptic shape it is necessary to increase the number of polygons and consequently the load on the processor in terms of vertex data processing and collision detection. Humans may be able to detect variation in surface texture with sub micro-meter accuracy. As a result, modelling objects in the simulation at this level of detail quickly reaches a limit above which an acceptable frame-rate cannot be maintained and is therefore not possible to deliver precise haptic reactions at a small scale using standard techniques.

Systems of the invention solve this problem by adding haptic detail to the flat surface of polygons via a two dimensional array of height offsets which can be easily authored as a grey-scale image using commonly available graphics tools. In standard graphics engines, polygons are defined by vertices. Each vertex contains its position in 3D space along with storage space for a two dimensional coordinate, referencing a point within an image, otherwise known as UV coordinates. With visual rendering the UV coordinates are used to enhance the appearance of the surface of the object.

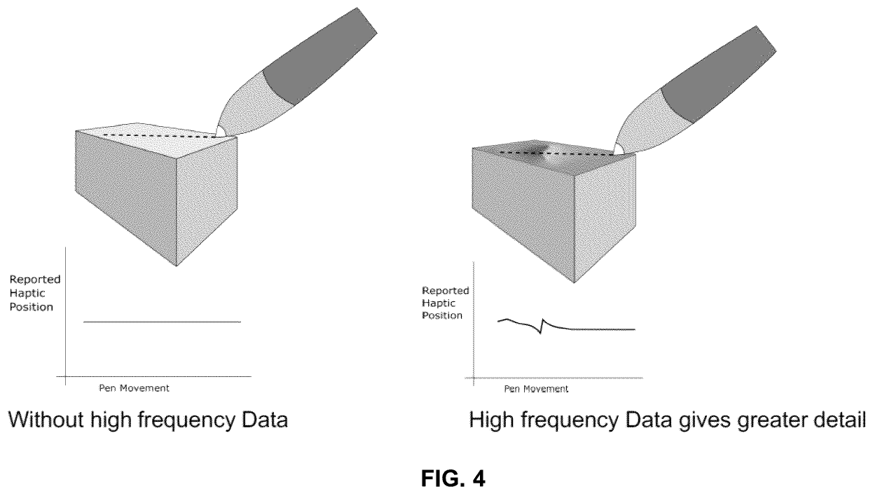

As illustrated in FIG. 4, systems of the invention use this technique in a novel way to enhance the available haptic detail to create previously unachievable levels of surface detail on a haptic object. As a result, the haptic detail matches the resolution of the visual detail and aligns haptic modelling with visually modelling. Further, the system may use other graphical techniques for giving surface detail to the haptic domain such as UV scrolling to provide impression of flow, bump map multiplication to give dynamic effects such as spatial areas of pulsing and other dynamic variations. Because the system does not require pre-programmed interactions for each tool/target/object combination, true free-form between the handheld component (haptic tool) and the virtual objects (targets) is achieved.

In non-limiting examples, the handheld component may be a wand, joystick, haptic glove, grasper, mouse or roller. In non-limiting examples, the virtual object may be a representation of one or more of a bone, a muscle, an organ, a blood vessel, blood, and a nerve. Also, in non-limiting examples, the simulated surgical procedure may be any procedure related to any discipline such as orthopedics, sports medicine, robotics, spinal/neuro procedures, ophthalmology, gene therapy, urology, interventional, endovascular, radiology, imaging, anesthesiology, regenerative therapies, surgical robots, and precision surgery. Accordingly, the invention provides for a suite of simulated surgical procedures and techniques for application in general and specific disciplines. For example, systems of the invention provide a suite of simulated surgical procedures and techniques that may be used for measurement and acquisition of universal skills required for surgeons regardless of discipline.

Further, systems of the invention include performant rendering of interior volumes of multi-part solids for seamless representation of dynamic object interaction and anatomical structures. Because the technology frees up control for the user and allows free-form interaction with the simulated subject matter, the invention addresses the associated challenges presented for graphical representation. For example, simulations must calculate and represent visual properties in milliseconds based on the user's actions. The systems of the invention disclose novel advances in the rendering of the interior volumes of multi-part solids, combining a voxel representation with a novel use of texture atlasing, enabling significant improvement in performance and efficiency. The result is the seamless representation of dynamic object interaction with anatomical structures made up of many different textures and tissues, that is hardware agnostic.

Systems of the invention may use texture atlasing to tri-planar mapping, an approach to modelling 3-dimensional (3D) visual graphics which results in higher fidelity and lower latency of visual elements. Within the domain of 3D computer graphics, tri-planar visual texture mapping is the conventional way to texture the meshes created by various adaptive mesh algorithms (marching cubes/tetrahedrons etc.). It creates a near seamless surface by applying a texture using coordinates projected along three axes and blending them according to the vertex normals. However, representation of multiple material types is not well supported using this method, often leaving a less than ideal visual representation or a downturn in rendering speed, such that, as the user penetrates the virtual object, each visual effect needs to be programmed and rendered into the space. This results in a significant increase in code complexity and requires the creation of multiple meshes which in turn require multiple draw calls to be made, increasing processor load.

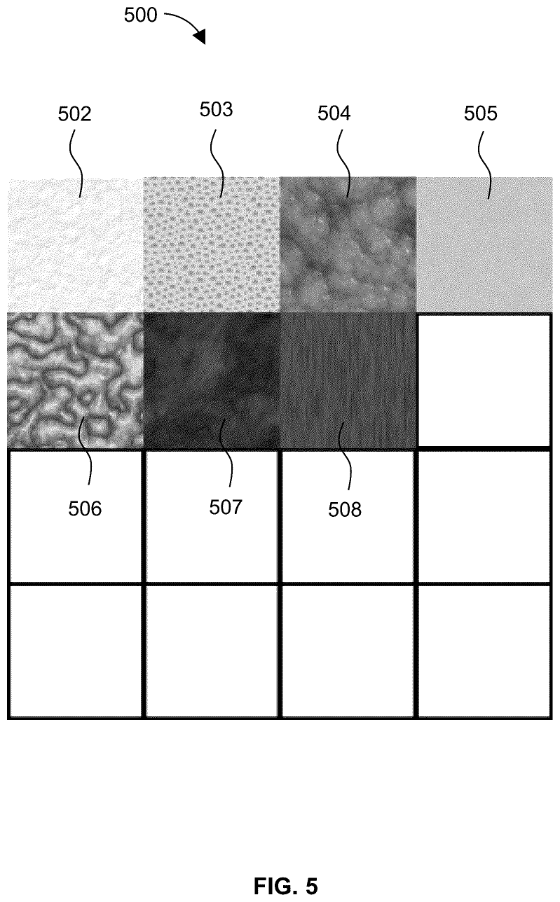

FIG. 5 illustrates an example of a texture atlas 500 where the textures for various tissues types have been combined according to one embodiment of the invention. Systems of the invention solve the problem of representing multiple material types by using the otherwise unused texture coordinates (also known as UV coordinates) to represent an offset into a texture atlas comprised of multiple surface textures to depict various materials. The materials may be, for example, any tissue types known in the medical field, e.g. muscle, bone, cartilage, nerve fiber, adipose tissue, skin, brain tissue, etc.). To illustrate, in the texture atlas 500 shown the tissue types include cortical bone 502, trabecular bone 503, fat or adipose tissue 504, epidermis or skin 505, brain tissue 506, blood 507, and muscle tissue 508. Each of the material types in the texture atlas corresponds to a unique set of coordinates (e.g., UV coordinates).

Hence, unused UV coordinates are repurposed to refer to a texture atlas comprising the various material types, thereby providing an efficient and streamlined method of applying textures to a virtual object in real-time. For example, UV coordinates associated with voxels of a virtual object may be identified and then used to locate textures within the texture atlas using offset values based on the UV coordinates. Once located, the textures may be mapped onto the voxels of the virtual object. This provides a solution to the technical problem of generating a virtual environment with virtual objects that have multiple textures in real-time.

In relation to tissue types, systems of the invention may use the tissue type data in the voxel representation to dynamically generate UV coordinates that can be stored in the mesh data.

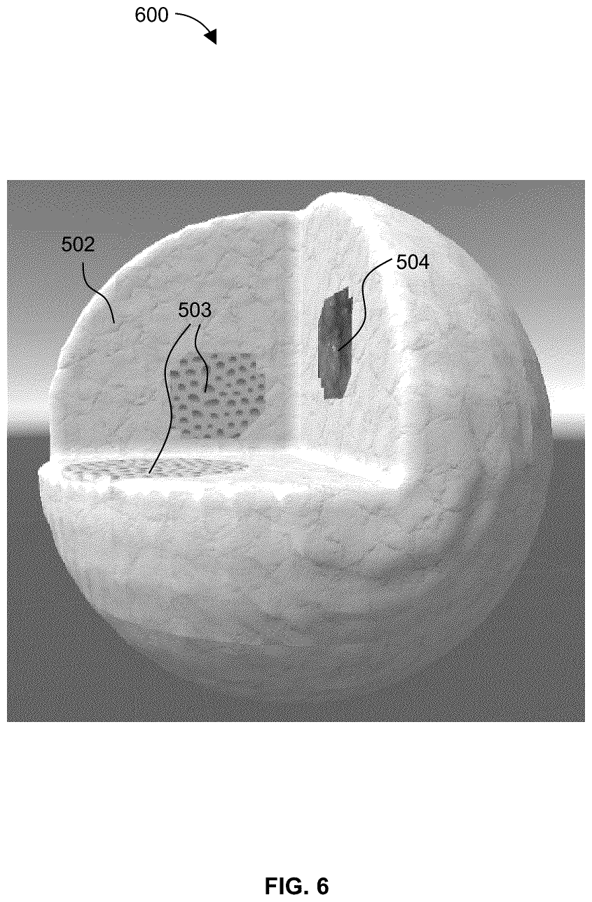

FIG. 6 shows a rendering of an object 600 of the mesh having multiple tissue types. The rendering of the model 600 or object is created with textures corresponding to cortical bone 502, trabecular bone 503, and fat tissue 504. The systems and methods disclosed herein may utilize a texture atlas to represent any visually distinct tissue type. The tissue type data in the voxel representation is used to dynamically generate UV coordinates that can be stored in the mesh data. When these are used in conjunction with a texture atlas, the triplanar shader can visually represent the different types of tissue that make up the model. This approach provides a way of delivering high visual fidelity, and enables the use of the voxel representation of complex multi-part solids that can be manipulated in real time. The use of a single texture atlas to represent multiple materials preserves rendering speed as limiting the materials to one texture means only one draw call is needed. Using UV coordinates to represent an offset into that texture still allows the use of computationally inexpensive triplanar mapping but also provides multiple surface support.

Accordingly, systems of the invention provide novel development processes for both 3-dimensional (3D) modelling and haptic feedback creation focused on methodologies to increase speed and quality of production and removing manual processes, which results in a surgical simulation experience capable of skills transfer via a realistic experience for rehearsal and assessment in a team environment, such that pre-human competence may be achieved.

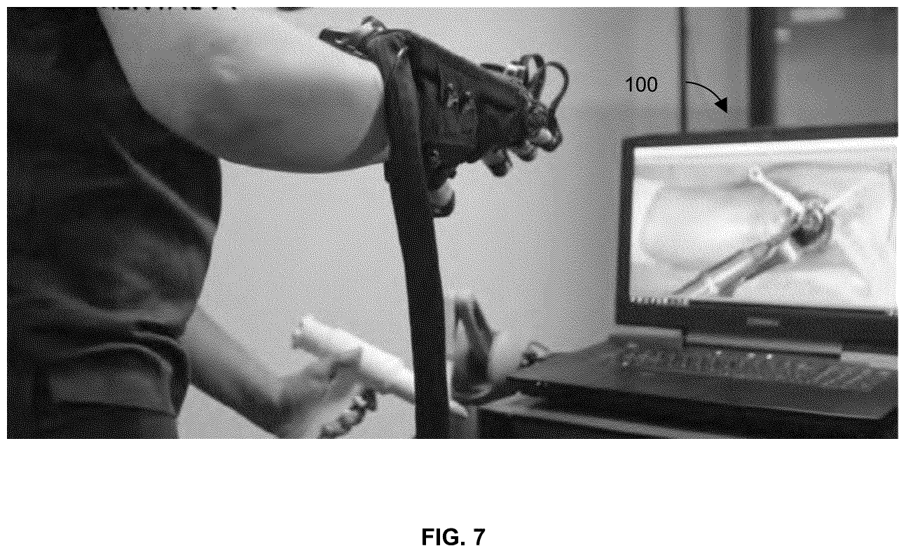

FIG. 7 shows a user interacting with digital content according to one embodiment of systems 100 of the invention. Systems and methods of the invention are hardware and software agnostic, providing seamless integration with various haptic and non-haptic input devices, for example Geomagic Touch, and Haptic Glove. The systems support both cutaneous and kinesthetic haptics, in the form of multiple handheld devices and gloves. The systems support current tethered headset product lines, as well as untethered headsets such as Oculus Quest 2 and HTC Focus 3.

### Multi-User Surgical Simulation System

Health care providers and physical hospitals have an ongoing need for training and skills development. The invention provides systems for the acceleration of skills acquisition via multi-user, remote, immersive medical training within a virtual reality (VR), augmented reality (AR), mixed reality (MR) and/or cross-reality (XR) simulated surgical environment. Simulation, in this context, is the practice to replace or amplify real experiences with guided experiences, immersive in nature, that evoke or replicate substantial aspects of the real world in a fully interactive manner. The systems of the invention allow for collaboratively developing situational awareness, and permit teams to remotely rehearse and practice in a low friction way by physically interacting with the virtual patient. Systems of the invention enable users to acquire the experience and knowledge of how it feels to perform a procedure correctly or incorrectly, building muscle memory, which is particularly important in advanced and precise surgical training.

In this context, virtual reality (VR) may be the computer-generated simulation of a three-dimensional image or environment that can be interacted with in a seemingly real or physical way. The interaction takes place using specific electronic hardware, for example, a headset or gloves. Augmented reality (AR) may be technology that superimposes a computer-generated image on a user's view of the real world, thus providing a composite view. Mixed reality (MR) may be a term used to describe the merging of a real-world environment and a computer-generated one. Physical and virtual objects may co-exist in mixed reality environments and interact in real time. Mixed reality that incorporates haptics may be referred to as Visuo-haptic mixed reality. Cross reality (XR) is immersive technology that brings physical objects into digital environments and takes digital objects into physical reality. XR includes hardware and software used to create virtual reality (VR), mixed reality (MR), augmented reality (AR) and cinematic reality (CR).

In one aspect, the invention discloses systems for providing a simulated surgical environment in which the system comprises a simulation platform configured to communicate and exchange data with one or more hand-held components and one or more wearable displays over a network, wherein the platform comprises a non-transitory computer-readable storage medium coupled to the processor and encoded with a computer program that causes the processor to generate and provide digital content comprising one or more simulated objects within a simulated surgical environment to be presented to multiple users interacting with the simulated surgical environment via associated hand-held components and wearable displays. The associated hand-held components and wearable displays are configured to provide haptic and visual feedback, respectively, to each of the multiple users within the simulated surgical environment. Further, the platform causes the processor to monitor actions of the multiple users to identify at least one of a user's field of view within the surgical environment and user interaction with a simulated object within the surgical environment based on physical user interaction with an associated hand-held component, and adjust output and synchronize sharing of digital content in real time across at least one of the hand-held component and wearable display associated with a given user in response to the actions of one or more of the users. This allows the multiple users to simultaneously interact within the simulated surgical environment via the hand-held components and wearable displays.

With reference to FIG. 2, illustrating one embodiment of an exemplary system 100 consistent with the present disclosure, the system 100 includes a simulation platform 103 embodied on an internet-based computing system/service. As shown, the system architecture backend may be a cloud based service 101. The system architecture may be multi-tenant.

The network 113 may represent, for example, a private or non-private local area network (LAN), personal area network (PAN), storage area network (SAN), backbone network, global area network (GAN), wide area network (WAN), or collection of any such computer networks such as an intranet, extranet or the Internet (i.e., a global system of interconnected network upon which various applications or service run including, for example, the World Wide Web). In alternative embodiments, the communication path between the simulation platform and/or the handheld components, wearable displays, and the cloud-based service, may be, in whole or in part, a wired connection.

The network may be any network that carries data. Non-limiting examples of suitable networks that may be used as network include Wi-Fi wireless data communication technology, the internet, private networks, virtual private networks (VPN), public switch telephone networks (PSTN), integrated services digital networks (ISDN), digital subscriber link networks (DSL), various second generation (2G), third generation (3G), fourth generation (4G) cellular-based data communication technologies, Bluetooth radio, Near Field Communication (NFC), the most recently published versions of IEEE 802.11 transmission protocol standards as of October 2018, other networks capable of carrying data, and combinations thereof. In some embodiments, network may be chosen from the internet, at least one wireless network, at least one cellular telephone network, and combinations thereof. As such, the network may include any number of additional devices, such as additional computers, routers, and switches, to facilitate communications. In some embodiments, the network may be or include a single network, and in other embodiments the network may be or include a collection of networks.

As shown, the simulation platform includes a computing system 105 comprising a storage medium 107 coupled to a processor 109 that causes the processor 109 to generate and provide digital content, as described above. As described in detail herein, the platform may dynamically adjust the generated digital content in response to data (feedback) received from the users and/or devices.

The simulation platform 100 may be configured to communicate and share data with one or more handheld components and one or more wearable displays associated with multiple users over a network. The system is hardware/software agnostic. Accordingly, the handheld devices and/or wearable displays may be embodied as any type of device for communicating with the simulation platform and cloud-based service, and/or other user devices over the network. In non-limiting examples, the handheld device may be a tethered or untethered haptic or non-haptic input device, such as the Geomagic Touch, and Haptic Glove. The platform supports both cutaneous and kinesthetic haptics, in the form of multiple handheld devices and gloves. Cutaneous, or fingertip, haptic interfaces stimulate the user's skin at the fingertip to emulate the sensation of touching a real object by stimulating RA, SAI, and SA2 type mechanoreceptors. Kinesthetic haptic interfaces provide feedback in the form of force sensations that stimulate both mechanical stimuli as well as stimuli related to the position and movement of the body. The wearable display may be video display glasses or headset, or smart glasses. The system supports current tethered headset product lines, as well as untethered headsets such as Oculus Quest 2 and HTC Focus 3. The devices may also provide audio feedback, for example via a microphone associated with the device or a standalone microphone. The devices may include motion sensors and/or location data. The motion sensors may be embodied as a combination of sensors within the device configured to capture a specific characteristic of the motion of the device or a specific characteristic of user movement of the device.

As disclosed herein, in some embodiments, the haptic and/or visual feedback may be computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined. Accordingly, systems of the invention provide for more degrees of freedom and different types of interaction, e.g. tactile as well as force feedback, audio, and visual feedback.

The system may capture data from a variety of sensors associated with the user devices, for example, location of the user within the simulated environment, a point of gaze of the user within the simulated environment, a field of view of the user within the simulated environment, as well as a physical setting and objects within the environment. The sensors may include one or more of a camera, motion sensor, and global positioning satellite (GPS) sensor.

As noted, systems of the invention are software and hardware agnostic and the platform may be configured to interact without limitation with devices that may be embodied as a computer, a desktop computer, a personal computer (PC), a tablet computer, a laptop computer, a notebook computer, a mobile computing device, a smart phone, a cellular telephone, a handset, a messaging device, a work station, a distributed computing system, a multiprocessor system, a processor-based system, and/or any other computing device configured to store and access data, and/or to execute software and related applications consistent with the present disclosure.

The platform provides a user interface or dashboard with which a user and/or administrators, such as institution managers, may interact. In some embodiments, the platform includes Learning Management System (LMS) features, such as user performance and progression, data export, recorded training modalities, and screen sharing for real-time skill assessment and proctoring. The platform may include administration views of data collected with analytics and report generation. In some embodiments, data may be exported to various LMS software for integration into other LMS platforms. As noted above, the platform may be configured to interact with various devices. Users may interact with the user interface/dashboard via an associated device. For example, the platform may include a web portal with which the user may interact with the dashboard/system via a mobile device, tablet, and/or desktop computer, such that the user interface may be embodied as a web application.

The computing system 105 may further include a display interface that forwards graphics, text, sounds, and other data input from the user devices for display on a display unit. The devices may include one or more integrated or ancillary devices for interacting with the system such as a keypad, touchscreen, microphone, camera, as well as other input components, including motion sensors, and the like. The display interface may generally display graphics and text, as well as provide a user interface (e.g., but not limited to graphical user interface (GUI)) through which a user may interact with the system, such as accessing and interacting with applications executed by the system.

The system 100 of the present disclosure may include, in some embodiments, computer systems, computer operated methods, computer products, systems including computer-readable memory, systems including a processor and a tangible, non-transitory memory configured to communicate with the processor, the tangible, non-transitory memory having stored instructions that, in response to execution by the processor, cause the system to perform steps in accordance with the disclosed principles, systems including non-transitory computer-readable storage medium configured to store instructions that when executed cause a processor to follow a process in accordance with the disclosed principles, etc.

The platform 103 includes a computing system 105 which incorporates one or more processors, such as processor 109. Processor 109 may be operably connected to the communication infrastructure and system architecture. The processor 109 may be embodied as a single or multi-core processor(s), digital signal processor, microcontroller, or other processor or processing/controlling circuit.

The computing system 105 further includes memory 107, such as random access memory (RAM), and may also include secondary memory. The memory 107 may be embodied as any type of device or devices configured for short-term or long-term storage of data such as, for example, memory devices and circuits, memory cards, hard disk drives, solid-state drives, or other data storage devices. Similarly, the memory 107 may be embodied as any type of volatile or non-volatile memory or data storage capable of performing the functions described herein.

Systems of the invention include and/or utilize one or more databases. In view of the disclosure provided herein, those of skill in the art will recognize that many databases are suitable for storage and retrieval of baseline datasets, files, file systems, objects, systems of objects, as well as data structures and other types of information described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database may be internet-based. In further embodiments, a database may be web-based. In still further embodiments, a database may be cloud computing-based. In other embodiments, a database may be based on one or more local computer storage devices.

As discussed above, the platform includes a haptic intelligence engine with unique algorithmic processing techniques for creating visual and haptic textures that provide a fully realistic computer simulated surgery experience. The platform provides a dynamic surgical simulation experience wherein the platform is capable of dynamically adjusting the progression and outcome of procedures and the surgical environment based on input data from the user.

In addition to the haptic intelligence engine, systems of the invention may include modules and submodules, for example, a data collection and management module, digital content creation, management, and distribution module, and various databases for storage of data, such as a user database for storing profiles and data of users and/or their associated devices. A data collection and management module may be configured to communicate and exchange data with each of the databases, as well as the other aspects/modules of the system.

In some embodiments, the simulation platform may be configured to determine a type and a degree of haptic feedback to be provided to one or more users via an associated hand-held component based on a user's physical interaction with an associated hand-held component and the type of simulated object. The simulation platform may be configured to adjust output of digital content to thereby mimic operation and/or movement of the simulated object. In some embodiments, the simulated object may be a surgical instrument. For example, in non-limiting examples, the surgical instrument may be an instrument used for cutting, dissecting, holding, grasping, occluding, clamping, retracting, exposing, improving vision, suturing, stapling, suctioning, aspirating, dilating, and probing. For example, in non-limiting examples, the surgical instrument may be an instrument used for cutting, dissecting, holding, grasping, occluding, clamping, retracting, exposing, improving vision, suturing, stapling, suctioning, aspirating, dilating, and probing. In non-limiting examples, the virtual surgical instrument may be a scalpel, a needle driver, a clamp, a clip applier, a surgical stapler, a retractor, a periosteal elevator, a rongeur, a nerve hook, a curette, an awl, a probe, a sagittal saw, a drill, a suture, a hammer, a finger, a laparoscopic instrument, an electrocautery, or a suctioning instrument.

As noted above, systems of the invention may be configured to generate a database comprising one or more types and degrees of haptic feedback and/or threshold value. A database, as described herein, may be configured to function as a user's learning tool or a lookup table to evaluate a user's performance, e.g., after a simulated surgery is completed. In some embodiments, types and degrees of haptic feedback are presented in a database so that a user may be able to identify whether a parameter of a specific subject falls within or outside of a threshold value. In some embodiments, the database may be stored on a server on the network, or stored locally with data backup provided by a server. In some embodiments the database may be stored locally on the apparatus or the system.

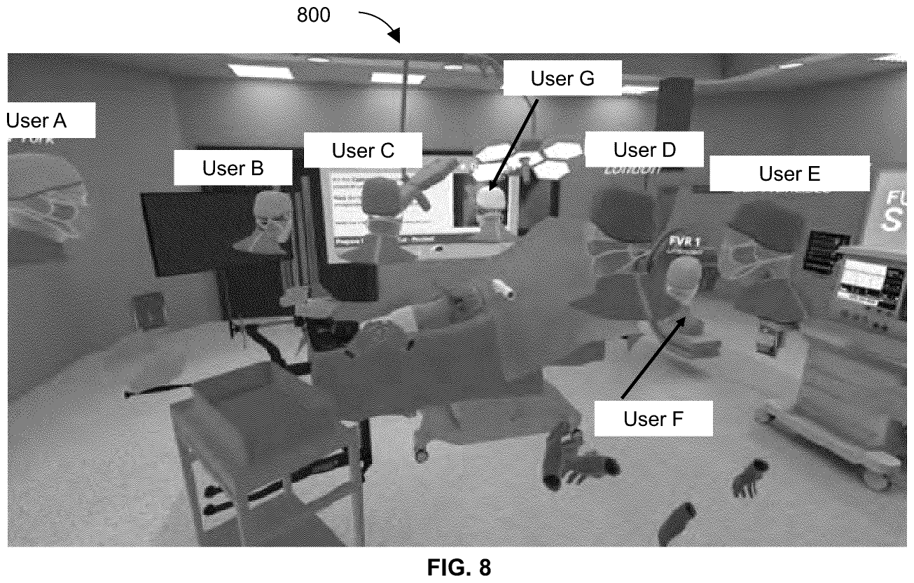

FIG. 8 illustrates a representation of one embodiment of the simulated surgical environment 800 of the invention in which multiple users are engaged within the environment. The rendering illustrates the field of view of an observer engaged within the simulated surgical environment. The platform may be configured to provide dynamic digital content comprising one or more simulated objects within a simulated surgical environment to be presented to multiple users interacting, via a user device, with the simulated surgical environment. As shown, the simulated surgical environment includes Users A-G collaborating within the surgical environment from remote locations. Accordingly, in some embodiments, the simulation platform may be configured to allow for one or more users to touch, manipulate, and/or modify the simulated object within the simulated surgical environment via an associated hand-held component. This feature allows for dynamic and variable interaction with patient cases within the simulated surgical environment that can involve many variations and pathologies, resulting in a true free-form interaction within the simulated surgical environment.

The platform allows for monitoring the actions of multiple users to identify at least one of a user's field of view within the surgical environment. The field of view, in the context of virtual reality, represents how much of the virtual world the user can see at once. As described above, the system uses novel processing techniques to identify user interaction with a simulated object within the surgical environment based on physical user interaction with an associated hand-held component. Data received from the physical interaction with an associated hand-held component may be input into the system and used to adjust digital, audio, and/or haptic output to synchronize sharing the digital content in real time across at least one hand-held component and wearable display associated with a user in in response to the actions of the user or users. This feature allows the multiple users to simultaneously interact within the simulated surgical environment via the hand-held components and wearable displays.

Multiple user capability allows more than one person to simultaneously interact within the simulated surgical environment for a realistic surgical collaboration experience. In the present context, depending on the surgical simulation provided and the particular use of the system, the user may include students, instructors, participants, observers, and the like. Accordingly, different users and devices in different locations may be joined in the simulated surgical environment by the platform.

For example, the surgical simulation may be used as full procedural training and rehearsal, team training, remote proctoring and monitoring, capability assessment. It is estimated that 43% of surgical errors involve failures in communication among personnel. The systems of the invention may be used as procedural practice for teams of medical personnel for both technical surgical skills and nontechnical skills such as teamwork and communication. Accordingly, the invention provides for simulating difficult, dynamic, and variable scenarios at low cost and high fidelity. The dynamic nature of the digital content provided by the platform delivers an environment in which users may develop difficult scenario pattern recognition while building the collaboration skills necessary to operate in the highly dynamic real world surgical environment.

Similarly, the systems of the present invention may be used as a classroom environment in which the instructor and associated course lesson may be provided to multiple students via the simulated surgical environment provided by the platform. Accordingly, systems of the invention provide for unlimited repetition of free-form interaction for learners to achieve muscle memory and ultimately mastery of surgical procedures.

In some embodiments, the simulation platform may be configured to track the actions of the one or more users, assess a performance of the one or more user based on the tracked actions, determine a performance rating for the actions of the user, and provide feedback to the one or more users associated with their performance rating. For example, the performance rating may be associated with a rating as compared to a level of “mastery” for the surgical procedure. The performance rating may be associated with a calculated simulated patient outcome as a result of the simulated surgical procedure.

In some embodiments, systems of the invention include an assessment module in which the outcome of a surgical procedure, in terms of success or failure may be evaluated. Accordingly, the performance rating may be associated with a comparison of the outcome of the simulated surgical procedure as compared to the real-world surgical procedure.

Systems of the invention provide for dynamic and variable interaction within the simulated surgical environment that can involve many variations and pathologies, resulting in a true free-form interaction within the simulated surgical environment. Because outcomes and results are not pre-determined, systems of the invention are configured to collect large amounts of user data that results in precision, real-time measurement related to decision-making in the simulated surgical environment. Data related to behavioral telemetry as well as performance outcomes may be collected and stored within the system. Data may be algorithmically analyzed at each step of the surgical objective level and used in real time to provide feedback to a user. Data may be collected and used to link what happens in the simulated surgical environment to patient outcomes in the real world. As a result, the invention provides data that for specific use cases that may be used for risk assessment, performance evaluation, and capability assessment.

Specifically, data measurement and interpretation may be used as part of a user's data dashboard to recognize mastery and to identify/predict individual learning curve development. Accordingly, systems of the invention may be utilized to provide insight into risk and capability through assessment metrics. Specifically, systems of the invention may be used to provide a risk assessment for surgical procedures and/or for individual mastery related to a surgical procedure. Accordingly, systems of the invention may be used to develop “pre-human” competence with the virtual reality skills transfer capable with the systems.

In some embodiments, the system provides for remote teleproctoring and telemonitoring. For example, in some embodiments, the multi-user capabilities of systems of the invention also provide for video streaming for remote collaboration integrated into the live operating room. In some embodiments, the actions of a first user are transmitted to the wearable display and the hand-held device of one or more other users. Systems of the invention provide for an augmented and/or cross reality experience by incorporating virtual reality simulations within the live operating room. For example, in some embodiments, cursers, constraints, gates, and guides may be added as virtual reality overlays in the live operating room to assist/support a live surgical procedure. In some embodiments, a user may hold a surgical tool to activate the simulation and thus feel the actions of another user who performs the simulated surgical procedure. Accordingly, in some embodiments, the simulated object comprises augmented reality (AR) content displayed as an overlay on a view of a real object in the live operating room.

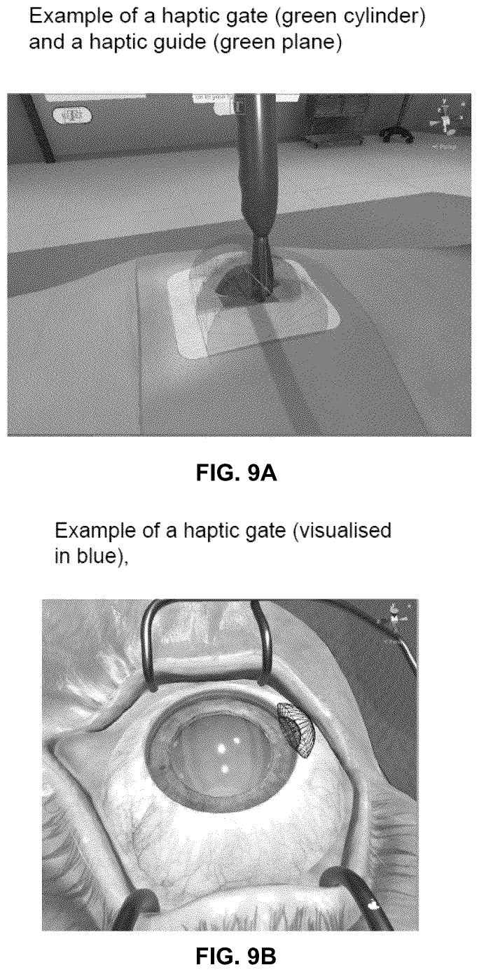

FIG. 9A and FIG. 9B illustrate renderings of embodiments of haptic gates and haptic guides of systems of the invention applied in a surgical simulation. FIG. 9A illustrates a haptic gate (green cylinder) and a haptic guide (green plane) applied as an overlay as part of a simulation of insertion of a surgical tool into an incision as support for a real life procedure in the live operating room. FIG. 9B illustrates a haptic gate (blue) overlayed as part of a simulation of precision eye surgery in support of the live surgery. The system also allows for the addition of angle and areas to create cursers, constraints, haptic gates, and guides to the live OR that can only exist in the virtual reality space. The system provides both visualization, for example how live hands are moving, plus pressure, angles, and artificial geometry to provide support for live OR procedures.

The systems of the invention are configured to collect and record significant performance and telemetry data. This data may be used for machine learning and product development at an anonymized level. Further, data insights via data measurement and interpretation are used within the platform to recognize “master” and to identify and predict learning curve development. In some embodiments, the data communicated and exchanged between the simulation platform and the one or more hand-held components and the one or more wearable displays comprises at least one of behavioral telemetry data, decision data, and performance outcome data associated with a user performing a surgical procedure in the live operating room. Behavioral telemetry data and decision data may be obtained from one or more users' interaction with the systems of the invention during a live or virtual surgical simulation. Performance outcome data may be obtained from the patient outcome in both the virtual simulated and live surgical procedure.

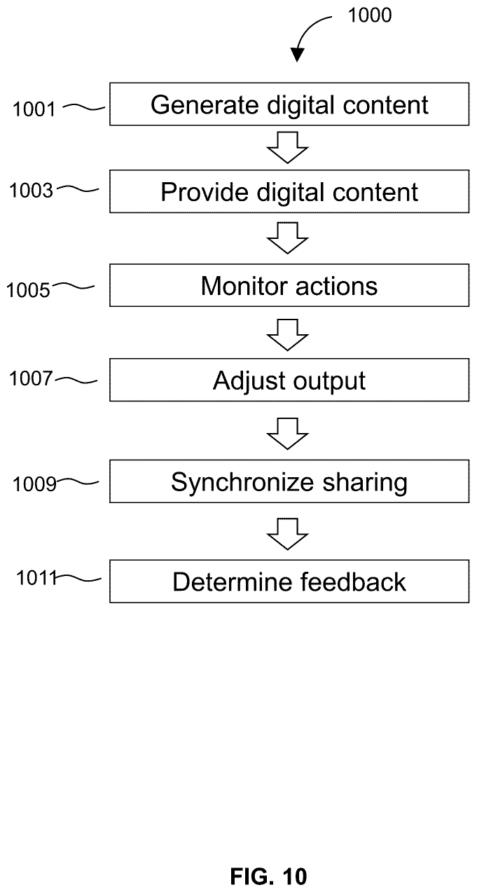

FIG. 10 illustrates one embodiment of methods of the invention for providing a simulated surgical environment. In one aspect, the method 1000 includes the steps generating 1001 and providing 1003, via a simulation platform, digital content comprising one or more simulated objects within a simulated surgical environment to be presented to multiple users interacting with the simulated surgical environment via associated hand-held components and wearable displays. The associated hand-held components and wearable displays are configured to provide haptic and visual feedback, respectively, to each of the multiple users within the simulated surgical environment. The method includes monitoring 1005, via the simulation platform, actions of the multiple users to identify at least one of a user's field of view within the surgical environment and user interaction with a simulated object within the surgical environment based on physical user interaction with an associated hand-held component. Further, the method includes adjusting 1007 output and synchronizing 1009 sharing of digital content, via the simulation platform, in real time across at least one of the hand-held component and wearable display associated with a given user in response to the actions of one or more of the users to thereby allow the multiple users to simultaneously interact within the simulated surgical environment via the hand-held components and wearable displays.

In some embodiments, the method further includes determining 1011, via the simulation platform, a type and a degree of haptic feedback to be provided to one or more users via an associated hand-held component based on a user's physical interaction with an associated hand-held component and the type of simulated objected.

As noted above, an exemplary system of methods of the invention includes a simulation platform embodied on an internet-based computing system/service. The system architecture backend may be a cloud based service 101. The system architecture may be multi-tenant. The network 113 may represent, for example, a private or non-private local area network (LAN), personal area network (PAN), storage area network (SAN), backbone network, global area network (GAN), wide area network (WAN), or collection of any such computer networks such as an intranet, extranet or the Internet (i.e., a global system of interconnected network upon which various applications or service run including, for example, the World Wide Web). In alternative embodiments, the communication path between the simulation platform and/or the handheld components, wearable displays, and the cloud-based service, may be, in whole or in part, a wired connection.

The network may be any network that carries data. Non-limiting examples of suitable networks that may be used as network include Wi-Fi wireless data communication technology, the internet, private networks, virtual private networks (VPN), public switch telephone networks (PSTN), integrated services digital networks (ISDN), digital subscriber link networks (DSL), various second generation (2G), third generation (3G), fourth generation (4G) cellular-based data communication technologies, Bluetooth radio, Near Field Communication (NFC), the most recently published versions of IEEE 802.11 transmission protocol standards as of October 2018, other networks capable of carrying data, and combinations thereof. In some embodiments, network may be chosen from the internet, at least one wireless network, at least one cellular telephone network, and combinations thereof. As such, the network may include any number of additional devices, such as additional computers, routers, and switches, to facilitate communications. In some embodiments, the network may be or include a single network, and in other embodiments the network may be or include a collection of networks.

The simulation platform of the methods may include a computing system that includes a storage medium coupled to a processor that causes the processor to generate and provide digital content, as described above. As described in detail herein, in some embodiments of the method, the platform may dynamically adjust the output of generated digital content in response to data (feedback) received from the users and/or devices to thereby mimic operation and/or movement of the simulated object.

The simulation platform of the methods may be configured to communicate and share data with one or more handheld components and one or more wearable displays associated with multiple users over a network. In some embodiments, the simulation platform may be configured to allow for the one or more users to touch, manipulate, and/or modify the simulated object within the simulated surgical environment via an associated hand-held component.

The system is hardware/software agnostic. Accordingly, the handheld devices and/or wearable displays used in the methods of the invention may be embodied as any type of device for communicating with the simulation platform and cloud-based service, and/or other user devices over the network. In non-limiting examples, the handheld device may be a tethered or untethered haptic or non-haptic input device, such as the Geomagic Touch, and Haptic Glove. The platform supports both cutaneous and kinesthetic haptics, in the form of multiple handheld devices and gloves. Cutaneous, or fingertip, haptic interfaces stimulate the user's skin at the fingertip to emulate the sensation of touching a real object by stimulating RA, SAI, and SA2 type mechanoreceptors. Kinesthetic haptic interfaces provide feedback in the form of force sensations that stimulate both mechanical stimuli as well as stimuli related to the position and movement of the body. The wearable display may be video display glasses or headset, or smart glasses. The system supports current tethered headset product lines, as well as untethered headsets such as Oculus Quest 2 and HTC Focus 3. The devices may also provide audio feedback, for example via a microphone associated with the device or a standalone microphone. The devices may include motion sensors and/or location data. The motion sensors may be embodied as a combination of sensors within the device configured to capture a specific characteristic of the motion of the device or a specific characteristic of user movement of the device.

In some embodiments of the method, the haptic and/or visual feedback may be computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined. Accordingly, methods of the invention provide for more degrees of freedom and different types of interaction, e.g. tactile as well as force feedback, audio, and visual feedback.

The systems of the method may capture data from a variety of sensors associated with the user devices, for example, location of the user within the simulated environment, a point of gaze of the user within the simulated environment, a field of view of the user within the simulated environment, as well as a physical setting and objects within the environment. The sensors may include one or more of a camera, motion sensor, and global positioning satellite (GPS) sensor.

As noted, systems of methods of the invention are software and hardware agnostic and the platform may be configured to interact without limitation with devices that may be embodied as a computer, a desktop computer, a personal computer (PC), a tablet computer, a laptop computer, a notebook computer, a mobile computing device, a smart phone, a cellular telephone, a handset, a messaging device, a work station, a distributed computing system, a multiprocessor system, a processor-based system, and/or any other computing device configured to store and access data, and/or to execute software and related applications consistent with the present disclosure.

The platform of the method provides a user interface or dashboard with which a user and/or administrators, such as institution managers, may interact. In some embodiments, the platform includes Learning Management System (LMS) features, such as user performance and progression, data export, recorded training modalities, and screen sharing for real-time skill assessment and proctoring. The platform may include administration views of data collected with analytics and report generation. In some embodiments, data may be exported to various LMS software for integration into other LMS platforms. As noted above, the platform may be configured to interact with various devices. Users may interact with the user interface/dashboard via an associated device. For example, the platform may include a web portal with which the user may interact with the dashboard/system via a mobile device, tablet, and/or desktop computer, such that the user interface may be embodied as a web application.

The computing system of the method may further include a display interface that forwards graphics, text, sounds, and other data input from the user devices for display on a display unit. The devices may include one or more integrated or ancillary devices for interacting with the system such as a keypad, touchscreen, microphone, camera, as well as other input components, including motion sensors, and the like. The display interface may generally display graphics and text, as well as provide a user interface (e.g., but not limited to graphical user interface (GUI)) through which a user may interact with the system, such as accessing and interacting with applications executed by the system.

Systems of methods of the present disclosure may include, in some embodiments, computer systems, computer operated methods, computer products, systems including computer-readable memory, systems including a processor and a tangible, non-transitory memory configured to communicate with the processor, the tangible, non-transitory memory having stored instructions that, in response to execution by the processor, cause the system to perform steps in accordance with the disclosed principles, systems including non-transitory computer-readable storage medium configured to store instructions that when executed cause a processor) to follow a process in accordance with the disclosed principles, etc.

As disclosed herein, the platform may include a computing system which incorporates one or more processors, such as processor. Processor may be operably connected to the communication infrastructure and system architecture. The processor may be embodied as a single or multi-core processor(s), digital signal processor, microcontroller, or other processor or processing/controlling circuit.

The computing system further includes memory, such as random access memory (RAM), and may also include secondary memory. The memory may be embodied as any type of device or devices configured for short-term or long-term storage of data such as, for example, memory devices and circuits, memory cards, hard disk drives, solid-state drives, or other data storage devices. Similarly, the memory may be embodied as any type of volatile or non-volatile memory or data storage capable of performing the functions described herein.

Systems of the invention include and/or utilize one or more databases. In view of the disclosure provided herein, those of skill in the art will recognize that many databases are suitable for storage and retrieval of baseline datasets, files, file systems, objects, systems of objects, as well as data structures and other types of information described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database may be internet-based. In further embodiments, a database may be web-based. In still further embodiments, a database may be cloud computing-based. In other embodiments, a database may be based on one or more local computer storage devices.

As discussed above, the platform of the method includes a haptic intelligence engine with unique algorithmic processing techniques for creating visual and haptic textures that provide a fully realistic computer simulated surgery experience. The platform provides a dynamic surgical simulation experience wherein the platform is capable of dynamically adjusting the progression and outcome of procedures and the surgical environment based on input data from the user.

In addition to the haptic intelligence engine, systems of the invention may include modules and submodules, for example, a data collection and management module, digital content creation, management, and distribution module, and various databases for storage of data, such as a user database for storing profiles and data of users and/or their associated devices. A data collection and management module may be configured to communicate and exchange data with each of the databases, as well as the other aspects/modules of the system.

In some embodiments of the method, the simulation platform may be configured to determine a type and a degree of haptic feedback to be provided to one or more users via an associated hand-held component based on a user's physical interaction with an associated hand-held component and the type of simulated object. The simulation platform may be configured to adjust output of digital content to thereby mimic operation and/or movement of the simulated object. In some embodiments, the simulated object may be a surgical instrument. For example, in non-limiting examples, the surgical instrument may be an instrument used for cutting, dissecting, holding, grasping, occluding, clamping, retracting, exposing, improving vision, suturing, stapling, suctioning, aspirating, dilating, and probing. In non-limiting examples, the virtual surgical instrument may be a scalpel, a needle driver, a clamp, a clip applier, a surgical stapler, a retractor, a periosteal elevator, a rongeur, a nerve hook, a curette, an awl, a probe, a sagittal saw, a drill, a suture, a hammer, a finger, a laparoscopic instrument, an electrocautery, or a suctioning instrument.

As disclosed herein, systems of methods of the invention may be configured to generate a database comprising one or more types and degrees of haptic feedback and/or threshold value. A database, as described herein, may be configured to function as a user's learning tool or a lookup table to evaluate a user's performance, e.g., after a simulated surgery is completed. In some embodiments, types and degrees of haptic feedback are presented in a database so that a user may be able to identify whether a parameter of a specific subject falls within or outside of a threshold value. In some embodiments, the database may be stored on a server on the network, or stored locally with data backup provided by a server. In some embodiments the database may be stored locally on the apparatus or the system.

The platform of methods of the invention allows for monitoring the actions of multiple users to identify at least one of a user's field of view within the surgical environment. The field of view, in the context of virtual reality, represents how much of the virtual world the user can see at once. As described above, the system uses novel processing techniques to identify user interaction with a simulated object within the surgical environment based on physical user interaction with an associated hand-held component. Data received from the physical interaction with an associated hand-held component may be input into the system and used to adjust digital, audio, and/or haptic output to synchronize sharing the digital content in real time across at least one hand-held component and wearable display associated with a user in in response to the actions of the user or users. This feature allows the multiple users to simultaneously interact within the simulated surgical environment via the hand-held components and wearable displays.

Multiple user capability allows more than one person to simultaneously interact within the simulated surgical environment for a realistic surgical collaboration experience. In the present context, depending on the surgical simulation provided and the particular use of the system, the user may include students, instructors, participants, observers, and the like. Accordingly, different people and devices in different locations may be joined in the simulated surgical environment by the platform.

For example, the surgical simulation may be used as full procedural training and rehearsal, team training, remote proctoring and monitoring, capability assessment. It is estimated that 43% of surgical errors involve failures in communication among personnel. The systems of the invention may be used as procedural practice for teams of medical personnel for both technical surgical skills and nontechnical skills such as teamwork and communication. Accordingly, the invention provides for simulating difficult, dynamic, and variable scenarios at low cost and high fidelity. The dynamic nature of the digital content provided by the platform delivers an environment in which users may develop difficult scenario pattern recognition while building the collaboration skills necessary to operate in the highly dynamic real world surgical environment.

Similarly, the systems of the present invention may be used as a classroom environment in which the instructor and associated course lesson may be provided to multiple students via the simulated surgical environment provided by the platform. Accordingly, systems of the invention provide for unlimited repetition of free-form interaction for learners to achieve muscle memory and ultimately mastery of surgical procedures.

In some embodiments, the simulation platform of the method may be configured to track the actions of the one or more users, assess a performance of the one or more user based on the tracked actions, determine a performance rating for the actions of the user, and provide feedback to the one or more users associated with their performance rating. For example, the performance rating may be associated with a rating as compared to a level of “mastery” for the surgical procedure. The performance rating may be associated with a calculated simulated patient outcome as a result of the simulated surgical procedure.

In some embodiments, systems of the invention include an assessment module in which the outcome of a surgical procedure, in terms of success or failure may be evaluated. Accordingly, the performance rating may be associated with a comparison of the outcome of the simulated surgical procedure as compared to the real-world surgical procedure.

The invention provides for dynamic and variable interaction with patient cases within the simulated surgical environment that can involve many variations and pathologies, resulting in a true free-form interaction within the simulated surgical environment Because outcomes and results are not pre-determined, methods and systems of the invention are configured to collect large amounts of user data that results in precision, real-time measurement related to decision-making in the simulated surgical environment. Data related to behavioral telemetry as well as performance outcomes may be collected and stored within the system. Data may be algorithmically analyzed at each step of the surgical objective level and used in real time to provide feedback to a user.

The data measurement and interpretation may be used as part of a user's data dashboard to recognize mastery and to identify/predict individual learning curve development. Accordingly, methods of the invention may be utilized to provide insight into risk and capability through assessment metrics. Specifically, methods of the invention may be used to provide a risk assessment for surgical procedures and/or for individual mastery related to a surgical procedure. Thus, methods of the invention may be used to develop “pre-human” competence with the virtual reality skills transfer capable with the systems.

In some embodiments, the multi-user capabilities of systems of the invention also provide methods for remote collaboration integrated into the live operating room. For example, in some embodiments, the actions of a first user are transmitted to the wearable display and the hand-held device of one or more other users. Methods of the invention provide for an augmented and/or cross reality experience by incorporating virtual reality simulations within the live operating room. For example, in some embodiments, cursers, constraints, gates, and guides may be added as virtual reality overlays in the live operating room to assist/support a live surgical procedure. In some embodiments, a user may hold a surgical tool to activate the simulation and thus feel the actions of another user who performs the simulated surgical procedure. Accordingly, in some embodiments, the simulated object comprises augmented reality (AR) content displayed as an overlay on a view of a real object in the live operating room.

In some embodiments, the data communicated and exchanged between the simulation platform and the one or more hand-held components and the one or more wearable displays comprises at least one of behavioral telemetry data, decision data, and performance outcome data associated with a user performing a surgical procedure in the live operating room. Behavioral telemetry data and decision data may be obtained from one or more users' interaction with the systems of the invention during a live or virtual surgical simulation. Performance outcome data may be obtained from the patient outcome in both the virtual simulated and live surgical procedure.

### Patient-Specific Surgical Simulations

The invention provides systems and methods for developing and simulating patient-specific and specific patient cases. Specifically, systems and methods of the invention provide for using data collected from various sources that may be related to a particular patient case to create a simulation that mirrors that particular case. The simulation may then be used for virtual surgical simulation of the specific case as a re-run of the case.

As disclosed herein, systems of the invention are configured to collect and record significant performance and telemetry data. For example, data associated with decisions taken, behavioral telemetry, and performance outcomes may be collected, analyzed, and used for content development and machine learning. This may be done at each step or surgical objective level and may be used in real time to provide feedback to a user as well as forming part of the user's data dashboard. Further, data insights via data measurement and interpretation are used within the platform to recognize “mastery” and to identify and predict learning curve development. As disclosed herein, systems of the invention may be used for training robotic medical devices. Accordingly, data collected may be used for assessing and evaluating robotic medical device learning, for example, for validation purposes.

The system may be configured to receive data for a specific patient case as input and to use this data to develop digital content related to the specific case. For example, data received may be used to generate a 3D simulated rendering of the particular anatomy associated with the case. Accordingly, the simulation platform may provide a full 3D virtual reality simulation, or re-run, of the specific case.

As noted above, systems of the invention provide for dynamic and variable interaction within the simulated surgical environment that can involve many variations and pathologies, resulting in a true free-form interaction within the simulated surgical environment. Because outcomes and results are not pre-determined, systems of the invention are configured to collect large amounts of user data that results in precise, real-time measurement related to decision-making in the simulated surgical environment. Data related to behavioral telemetry as well as performance outcomes may be collected and stored within the system. Data may be algorithmically analyzed at each step of the surgical objective level and used in real time to provide feedback to a user.

In some aspects, the invention discloses a system for providing a patient-specific simulated surgery. The system includes a simulation platform as described herein. As described, the simulation platform may be configured to communicate and exchange data with one or more handheld components and one or more wearable displays over a network. The platform includes a non-transitory computer-readable storage medium coupled to the processor and encoded with a computer program that causes the processor to receive data of a patient that represents a completed, real world surgical procedure of the patient. Further, the computer program causes the processor to generate and provide digital content comprising one or more simulated objects within a simulated surgical environment associated with the real world surgical procedure undergone by the patient, which may be presented to one or more users interacting with the simulated surgical environment via associated hand-held components and wearable displays. The simulated content may be based, at least in part, on the data received from the completed, real-world surgical procedure of the patient.

The data may be related to one or more surgical scenarios, or a combination of scenarios, and may be used to create a specific surgical simulation based on the data for the case or cases. Accordingly data from various sources may be input into the system and used to develop content of a specific use case. This content may be used to re-run the case as a virtual simulation. The case may be a particularly difficult presentation or a procedure in which an unusual event occurred during the procedure. The simulation may be performed after the actual case is conducted, thus presenting the opportunity for a simulated rerun or re-play of a specific case.

In some embodiments, the digital content comprises at least one of virtual reality (VR) content, mixed reality (MR), augmented reality (AR), and cross reality (XR) content. For example, the digital content may be a virtual three-dimensional (3D) model of a patient to undergo a surgical procedure.

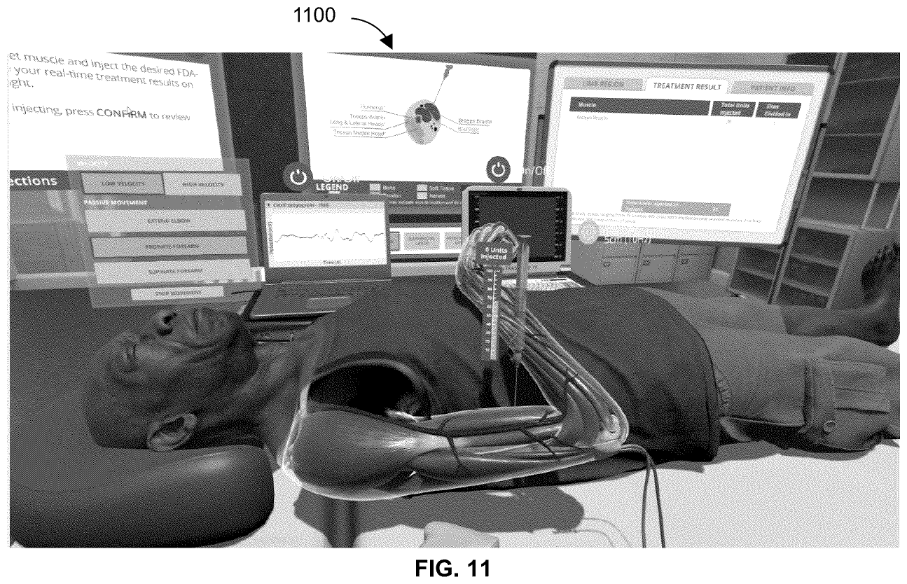

FIG. 11 illustrates one embodiment of digital content 1100 of systems of the invention depicting a virtual 3D model of a patient to undergo a surgical procedure. The virtual model of the patient may be created by the simulation engine using anonymized data used as input into the system. Data received from the patient case may be used to create any virtual patient or variables related to the patent or case, for example, size, shape, or gender. By integrating real patient data into the simulations, systems of the invention may be used to create simulations of patient-specific cases for rehearsal and training on individual surgeries.

In some embodiments, the digital content may be based, at least in part, on data selected from the group consisting of: data received via a standard protocol for the management and transmission of real medical images and related data; real behavioral telemetry data from a surgical procedure; real performance outcome data from a surgical procedure; and real decision data from a surgical procedure. As noted previously, data from a particular case may be imported into the system for development of the digital content, and used to create a simulation that mirrors that particular case. As discussed above, in some embodiments, the simulation platform includes a simulation engine configured to create digital content for the simulated surgical procedure.

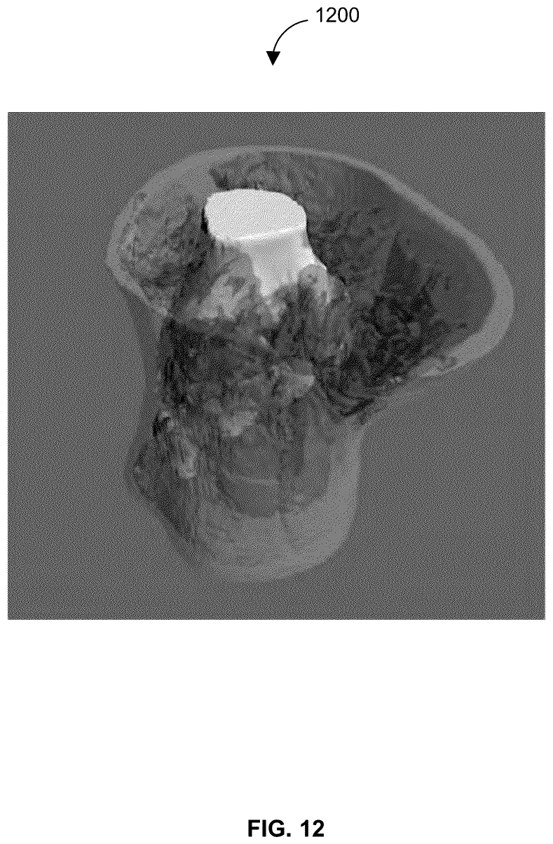

FIG. 12 shows digital content 1200 of a knee according to one embodiment of systems of the invention developed from specific-patient scan data imported into one embodiment of systems of the invention. In some embodiments, the content may be created using data received via a standard protocol for the management and transmission of medical images and related data. For example, the standard protocol may be Digital Imaging and Communications in Medicine (DICOM). The DICOM data may be any data. In non-limiting examples, the data may be one or more of scan data from a magnetic resonance imaging (MRI) scan, a computed tomography (CT), computed axial tomography (CAT) scan, positron emission tomography (PET), and/or an ultrasound scan.

Further, in some embodiments, the simulation platform of the system may be configured to receive scan data related to one or more surgical scenarios, segment the scan data, apply anatomical landmarks, apply one or more haptic values to the data, apply a templating algorithm to generate a 3D model of an anatomy, and parameterize the anatomy to create a simulated patient requiring a surgical procedure. For example, the system may be configured to recognize anatomy from a 2D view to parameterize the anatomy.

As noted above, in some embodiments of systems of the invention, the simulation platform may be configured to monitor actions of one or more users to identify at least one of a user's field of view within the surgical environment and user interaction with a simulated object within the surgical environment based on physical user interaction with an associated hand-held component. Further, the simulation platform may be configured to adjust output of digital content across at least one of the hand-held component and wearable display associated with a given user in response to the actions of one or more of the users. For example, and without limitation, the system may be configured to monitor user actions that impact the outcome of the simulated surgical procedure as compared to the outcome of the real-world surgical procedure being simulated. The system may be configured to provide a performance rating may be associated with a comparison of the outcome of the simulated surgical procedure as compared to the real-world surgical procedure.

The haptic and/or visual feedback to be provided to a given user via an associated hand-held component and wearable display may be computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, such that the algorithm establishes a resultant effect of the interaction with the digital content that is not pre-determined.

As noted above, in non-limiting examples, the surgical instrument may be an instrument used for cutting, dissecting, holding, grasping, occluding, clamping, retracting, exposing, improving vision, suturing, stapling, suctioning, aspirating, dilating, and probing. For example, in non-limiting examples, the surgical instrument may be an instrument used for cutting, dissecting, holding, grasping, occluding, clamping, retracting, exposing, improving vision, suturing, stapling, suctioning, aspirating, dilating, and probing. In non-limiting examples, the virtual surgical instrument may be a scalpel, a needle driver, a clamp, a clip applier, a surgical stapler, a retractor, a periosteal elevator, a rongeur, a nerve hook, a curette, an awl, a probe, a sagittal saw, a drill, a suture, a hammer, a finger, a laparoscopic instrument, an electrocautery, or a suctioning instrument.

In other aspects, the invention discloses a method for providing a patient-specific surgical environment.

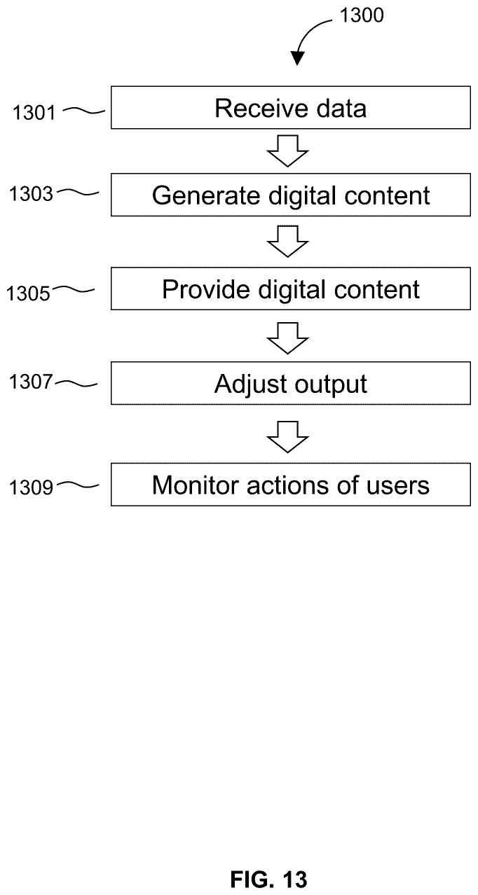

FIG. 13 illustrates one embodiment of a method 1300 of the invention for providing a patient-specific surgical environment. As illustrated, the method 1300 includes the steps of receiving 1301, via a simulation platform, data of a patient that represents a completed, real world surgical procedure of the patient. Further, the method includes generating 1303 digital content via the simulation platform, and providing 1305 the digital content to one or more users interacting with the simulated surgical environment via associated hand-held components and wearable displays. The digital content may include one or more simulated objects within a simulated surgical environment associated with the real world surgical procedure undergone by the patient, and based, at least in part, on the data received from the completed, real-world surgical procedure of the patient.

Further, the method may include the step of monitoring 1309, via the simulation platform, user actions within the simulated environment. The method may include adjusting 1307 the output based on, for example, user actions within the simulated environment. In specific embodiments, the method monitors user actions that impact the outcome of the simulated surgical procedure as compared to the outcome of the real-world surgical procedure. User actions that impact the outcome of the simulated surgical procedure as compared to the real-world procedure may be actions that provide for an improved outcome as compared to the outcome of the real-world surgical procedure. Additionally and alternatively, the user actions may be actions that cause an inferior patient outcome as compared to the outcome of the real-world surgical procedure. The digital content of the methods may be one or more of VR content, AR content, MR content, and/or cross reality content. The digital content may be, for example, virtual three-dimensional (3D) model of a patient to undergo a surgical procedure.

In some embodiments of the methods, the digital content may be based, at least in part, on data selected from the group consisting of: data received via a standard protocol for the management and transmission of real medical images and related data; real behavioral telemetry data from a surgical procedure; real performance outcome data from a surgical procedure; and real decision data from a surgical procedure. For example, the data may be received from a standard protocol for the management and transmission of real medical images such as DICOM.

The DICOM data may be any data. In non-limiting examples, the data may be one or more of scan data from a magnetic resonance imaging (MRI) scan, a computed tomography (CT), computed axial tomography (CAT) scan, positron emission tomography (PET), and/or an ultrasound scan.

Further, in some embodiments of the method, the simulation platform of the system may be configured to receive scan data related to one or more surgical scenarios, segment the scan data, apply anatomical landmarks, apply one or more haptic values to the data, apply a templating algorithm to generate a 3D model of an anatomy, and parameterize the anatomy to create a simulated patient requiring a surgical procedure. For example, the system may be configured to recognize anatomy from a 2D view to parameterize the anatomy.

As noted above, in some embodiments of systems of the invention, the simulation platform may be configured to monitor actions of one or more users to identify at least one of a user's field of view within the surgical environment and user interaction with a simulated object within the surgical environment based on physical user interaction with an associated hand-held component. Further, the simulation platform may be configured to adjust output of digital content across at least one of the hand-held component and wearable display associated with a given user in response to the actions of one or more of the users. For example, and without limitation, the system may be configured to monitor user actions that impact the outcome of the simulated surgical procedure as compared to the outcome of the real-world surgical procedure being simulated. The system may be configured to provide a performance rating that may be associated with a comparison of the outcome of the simulated surgical procedure as compared to the real-world surgical procedure.

The haptic and/or visual feedback to be provided to a given user via an associated hand-held component and wearable display may be computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, such that the algorithm establishes a resultant effect of the interaction with the digital content that is not pre-determined.

As disclosed herein, in non-limiting examples, the surgical instrument may be an instrument used for cutting, dissecting, holding, grasping, occluding, clamping, retracting, exposing, improving vision, suturing, stapling, suctioning, aspirating, dilating, and probing. For example, in non-limiting examples, the surgical instrument may be an instrument used for cutting, dissecting, holding, grasping, occluding, clamping, retracting, exposing, improving vision, suturing, stapling, suctioning, aspirating, dilating, and probing. In non-limiting examples, the virtual surgical instrument may be a scalpel, a needle driver, a clamp, a clip applier, a surgical stapler, a retractor, a periosteal elevator, a rongeur, a nerve hook, a curette, an awl, a probe, a sagittal saw, a drill, a suture, a hammer, a finger, a laparoscopic instrument, an electrocautery, or a suctioning instrument.

### Specific Patient Surgical Simulations

Systems and methods of the invention provide for the simulation of specific-patient surgical procedures. Accordingly, the invention provides for using patient data to create a simulated surgical procedure for a specific patient that may be used to rehearse an upcoming or proposed procedure for that patient before the actual real-world surgical procedure. As such, data received related to the patient may be used as input into the system to create a specific patient case. This specific patient case may be used for unlimited rehearsals before the actual surgery to improve patient outcomes. Modeling the specific patient allows for surgical preparation and run through before the actual surgical procedure. Accordingly the content generated and provided by the system may be used for pre-operating room planning and to train for pre-human competence before the actual real-world surgical procedure.

The system may be configured to use data, digital or analog, collected from an actual patient. For example, the data may be digital data collected using medical imaging techniques such as X-ray imaging, CAT scan, magnetic resonance imaging (MRI), ultrasonic imaging, endoscopic imaging, tactile imaging, thermographic imaging, photographic imaging, positron emission tomography (PET), single photon emission computed tomography imaging (SPECT), elastographic imaging, photoacoustic imaging, tomographic imaging, echocardiographic imaging, functional near infrared imaging or magnetic particle imaging. The digital imaging data of a patient can be produced by taking at least one X-ray or CAT scan image of the area where the surgery is to be performed and then shown in the virtual or simulated environment.

Alternatively and/or additionally, other imaging data may be used such as from an MM or scanned image of a surface model of the surgical site. This data can be used to virtually reconstruct the patient's actual surgical field to be used in the virtual simulation. Such data may include a specific patient's bone structure and formation or bone tissue. In another example, this data may be useful for creating a prosthesis for the patient after the surgery has concluded. The actual digital imaging data may be stored within a database. The database may be a local database (e.g., stored on a local server) or a remote network or cloud database.

The database may include data relating to nonphysical properties or other medical information of a specific patient, such as a patient's medical history, known allergies, and illnesses. The database may include information pertaining to a patient's treatment plan or procedure. As disclosed herein, a processor may access the data contained within the database and provide the user access to this data. A user may access and upload a patient's digital data to the system and use the data to practice a specific patient's planned surgery within the simulated surgical environment.

In one aspect, the invention discloses a system for providing a specific-patient simulated surgery. The system includes a simulation platform, as disclosed herein, configured to communicate and exchange data with one or more handheld components and one or more wearable displays over a network. The platform includes a non-transitory computer-readable storage medium coupled to the processor and encoded with a computer program. The computer causes the processor to receive data of a patient prior to the patient undergoing a surgical procedure; and generate and provide digital content comprising one or more simulated objects within a simulated surgical environment associated with a simulated surgical procedure that is to be undergone by the patient prior to the patient undergoing the actual surgical procedure. The digital content may be presented to one or more users interacting with the simulated surgical environment via associated hand-held components and wearable displays. The digital content may be based, at least in part, on data received from the specific patient requiring the surgical procedure.

In specific embodiments, the system may be configured to monitor user actions. The user actions, for example, may be actions that impact the outcome of the simulated surgical procedure as compared to the outcome of the real-world surgical procedure. User actions that impact the outcome of the simulated surgical procedure as compared to the real-world procedure may be actions that provide for an improved outcome as compared to the outcome of the real-world surgical procedure. Additionally and alternatively, the user actions may be actions that cause an inferior patient outcome as compared to the outcome of the real-world surgical procedure.

The digital content may be one or more of VR content, AR content, MR content, and/or cross reality content. The digital content may be, for example, virtual three-dimensional (3D) model of a patient to undergo a surgical procedure.

As discussed herein, the digital content may be based, at least in part, on data selected from the group consisting of: data received via a standard protocol for the management and transmission of real medical images and related data; real behavioral telemetry data from a surgical procedure; real performance outcome data from a surgical procedure; and real decision data from a surgical procedure. In specific embodiments, the data may be received via a standard protocol for the management and transmission of real medical images and related data, such as DICOM.

In some embodiments, the simulation platform includes a simulation engine that may be configured to create the digital content for the simulated surgical procedure. Further, the simulation platform may be configured to receive scan data related to one or more surgical scenarios; segment the scan data; apply anatomical landmarks; apply one or more haptic values to the data; apply a templating algorithm to generate a 3D model of an anatomy; and parameterize the anatomy to create a simulated patient requiring a surgical procedure. For example, the system may be configured to recognize anatomy from a 2D view to parameterize the anatomy.

Further, the simulation platform may be configured to adapt the digital content in response to user actions in the virtual surgical environment; track actions of the user during the simulated surgical procedure; assess a performance of the user based on the tracked actions; determine a performance rating for the actions of the user; provide feedback to the user; and determine a pre-human competence for the surgical procedure. For example, the performance rating may be associated with a rating as compared to a level of “mastery” for the surgical procedure. The performance rating may be associated with a calculated simulated patient outcome as a result of the simulated surgical procedure.

The haptic and/or visual feedback to be provided by the system to a given user via an associated hand-held component and wearable display may be computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined. Accordingly, the invention provides for a free-form interaction with the simulated surgical environment.

As disclosed herein, in non-limiting examples, the surgical instrument may be an instrument used for cutting, dissecting, holding, grasping, occluding, clamping, retracting, exposing, improving vision, suturing, stapling, suctioning, aspirating, dilating, and probing. For example, in non-limiting examples, the surgical instrument may be an instrument used for cutting, dissecting, holding, grasping, occluding, clamping, retracting, exposing, improving vision, suturing, stapling, suctioning, aspirating, dilating, and probing. In non-limiting examples, the virtual surgical instrument may be a scalpel, a needle driver, a clamp, a clip applier, a surgical stapler, a retractor, a periosteal elevator, a rongeur, a nerve hook, a curette, an awl, a probe, a sagittal saw, a drill, a suture, a hammer, a finger, a laparoscopic instrument, an electrocautery, or a suctioning instrument.

In another aspect, the invention discloses a method for providing patient-specific simulated surgery.

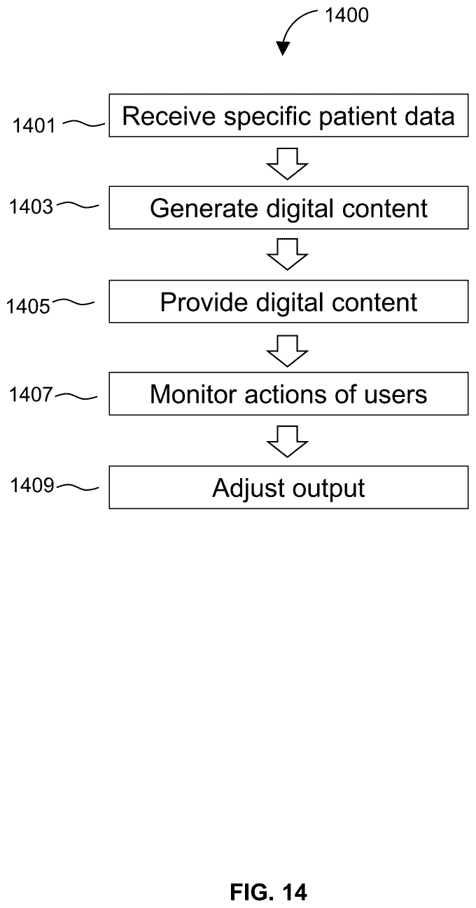

FIG. 14 illustrates an embodiment of a method 1400 of the invention for providing patient-specific simulated surgery. The method includes the steps of receiving 1401, via a simulation platform as disclosed herein, data of a patient prior to the patient undergoing a surgical procedure. The method further includes generating 1403 and providing 1405, via the simulation platform, digital content comprising one or more simulated objects within a simulated surgical environment associated with a simulated surgical procedure that is to be undergone by the patient prior to the patient undergoing the actual surgical procedure. This digital content may be presented to one or more users interacting with the simulated surgical environment via associated hand-held components and wearable displays. The digital content may be based, at least in part, on data received from the specific patient requiring the surgical procedure.

The simulation platform may be configured to monitor 1407 actions of one or more users to identify at least one of a user's field of view within the surgical environment and user interaction with a simulated object within the surgical environment based on physical user interaction with an associated hand-held component. Further, the system, i.e. the simulation platform may be configured to adjust 1409 output and synchronize sharing of digital content in real time across at least one of the hand-held component and wearable display associated with a given user in response to the actions of one or more of the users.

For example, monitoring 1407 user actions in the simulated surgical environment may be user actions that impact the outcome of the simulated surgical procedure for the specific patient. In this way, the simulated surgical procedure may be practiced or rehearsed as necessary for pre-procedural training before the actual real-world procedure for the patient.

In some embodiments of the method, the digital content comprises at least one of virtual reality (VR) content, mixed reality (MR), augmented reality (AR), and cross reality (XR) content. For example, the digital content may be a virtual three-dimensional (3D) model of a patient to undergo a surgical procedure. The digital content may be based, at least in part, on data selected from the group consisting of: date received via a standard protocol for the management and transmission of real medical images and related data; real behavioral telemetry data from a surgical procedure; real performance outcome data from a surgical procedure; and real decision data from a surgical procedure.

As disclosed herein, the digital content of the method may be based, at least in part, on data selected from the group consisting of: data received via a standard protocol for the management and transmission of real medical images and related data; real behavioral telemetry data from a surgical procedure; real performance outcome data from a surgical procedure; and real decision data from a surgical procedure. In specific embodiments, the data may be received via a standard protocol for the management and transmission of real medical images and related data, such as DICOM.

In some embodiments of the method, the simulation platform includes a simulation engine that may be configured to create the digital content for the simulated surgical procedure. Further, the simulation platform may be configured to receive scan data related to one or more surgical scenarios; segment the scan data; apply anatomical landmarks; apply one or more haptic values to the data; apply a templating algorithm to generate a 3D model of an anatomy; and parameterize the anatomy to create a simulated patient requiring a surgical procedure. For example, the system may be configured to recognize anatomy from a 2D view to parameterize the anatomy.

Further, the simulation platform may be configured to adapt the digital content in response to user actions in the virtual surgical environment; track actions of the user during the simulated surgical procedure; assess a performance of the user based on the tracked actions; determine a performance rating for the actions of the user; provide feedback to the user; and determine a pre-human competence for the surgical procedure. For example, the performance rating may be associated with a rating as compared to a level of “mastery” for the surgical procedure. The performance rating may be associated with a calculated simulated patient outcome as a result of the simulated surgical procedure.

The haptic and/or visual feedback to be provided by the system to a given user via an associated hand-held component and wearable display may be computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined. Accordingly, the invention provides for a free-form interaction with the simulated surgical environment.

### Simulated Soft Tissue Suturing

The systems and methods of the invention provide novel approaches to solving technical challenges unique to simulating suturing, namely the interaction between the thread and the soft tissue. Collision handling between the two bodies is complicated and often unstable. The invention uses a novel algorithm to model the movement of the suture material as it is pulled through the soft tissue by the user, thus manipulating the rope and soft body meshes, and runtime.

Specifically, the invention provides novel solutions to the challenges related to position-based solvers for soft tissue simulation, namely the behavior of the tissue when large displacements are applied, via either prodding or grabbing by the user. This is due to the particle nature of the simulation. The invention overcomes these challenges by implementing a novel collision detection algorithm as an additional layer on the existing deformable mesh.

Further, the invention addresses the challenge of stability of the simulation, specifically, the low stability of collision handling between simulated ropes and soft tissue at a low scale. This is particularly important for simulating an interaction such as suturing, which is often performed on a very localized area and therefore requires high accuracy. The invention solves this problem by using algorithms defining objects at various scales, generating a realistic perspective of the environment while keeping the suture thread at a scale at which it behaves in a reliably stable manner.

These and other novel solutions of the invention provide a free-form experience, with multiple customizable settings, not achievable with contemporary systems. Systems and methods of the invention provide for interactive, real-time simulation encompassing realistic soft tissue and thread behavior, for example interactions involving continuous and interrupted suturing. A continuous or uninterrupted suture may be defined as a kind of suture that is made with a single strand of suture material. Continuous sutures are one with a series of stitches but they are not individually knotted. Interrupted suturing involves individual stitches that are placed and tied individually, and therefore not connected. As described in more detail herein, systems and methods of the invention provide for surgical knot-tying, for example during interrupted suturing.

Systems and methods of the invention solve the challenges of handing multiple complex interactions between soft tissue, suture thread, surgical instruments, and a user's hands. The realistic, high fidelity soft tissue suturing simulation provided by the system takes into account that during real world soft tissue suturing, the environment is dynamic, with various levels of movement. Further, the actual soft tissue suturing is performed on a micro scale. Systems of the invention are able to replicate these dynamics using novel algorithms that simultaneously simulate the behavior of different tissue types, in addition to the behavior of the surgical instrument, the suture material, and the surgeon's hands.

The movement and deformation of both the thread and the tissue may be simulated using Position Based Dynamics, for example as described in Müller, 2006, Position Based Dynamics, 3rd Workshop in VR Inter and Phys Sim, incorporated by reference in its entirety herein. Systems and methods of the invention model movement and deformation of both the thread and the tissue as a collection of particles controlled by a set of various constraints. The surgical instruments are controlled by the simulation platform, as an abstracted interface between the various hardware. Accordingly, the systems of the invention are hardware agnostic.

Interaction between the soft tissue and surgical tools includes, but is not limited to, prodding, grabbing, piercing and pulling. The system provides algorithmic calculations to simulate the physics of different types of soft tissue, including flesh, simulation of the suture, simulation of the needle with the suture attached, simulation of the needle and suture material going through the tissue, the simulation of the interaction of the surgical tools and the surgeon's hands within the simulation.

For example, the system allows the user to prod the soft tissue and experience a realistic deformation response. The system allows the user to also grab the tissue and stretch it. Accordingly, in a non-limiting example, systems of the invention provide for simulating a thread fixed at one end to the soft tissue or a rigid, immovable object, or leaving it unfixed.

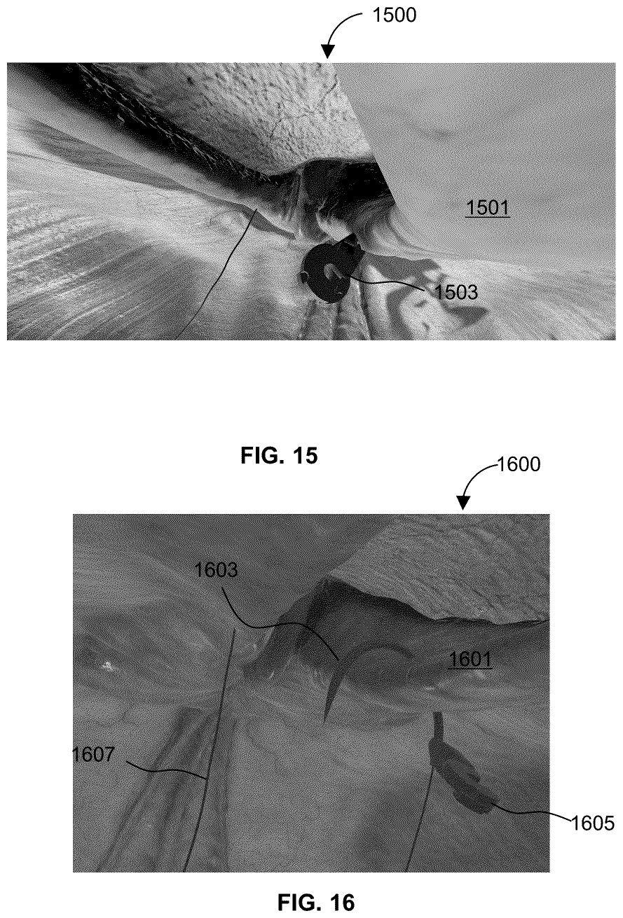

FIG. 15 shows digital content 1500 of simulated soft tissue suturing according to one embodiment of the invention, in which the user has grabbed the particular tissue 1501 using tissue forceps 1503 to stretch it. The ability to grab and stretch the simulated soft tissue provides a more usable and realistic experience during simulated surgical suturing.

FIG. 16 shows digital content 1600 illustrating the user piercing the particular soft tissue 1601 with an instrument such as a curved needle 1603 according to one embodiment of the invention. The needle 1603 may be held by the user with a tool, such as surgical forceps 1605.

Piercing of the soft tissue may be simulated using the collision detection system of the invention. This may trigger constraining the needle along a path while it is inside the soft tissue. For example, a curved needle may be restricted to follow a circular path around its center point, but with an allowable range of movement. This enables manipulation of the soft tissue if the user tries to pull the needle while inside the anatomy as is common when suturing with this type of needle. The content provides for the interaction of the suture 1607 with the needle 1603 and the tissue 1601.

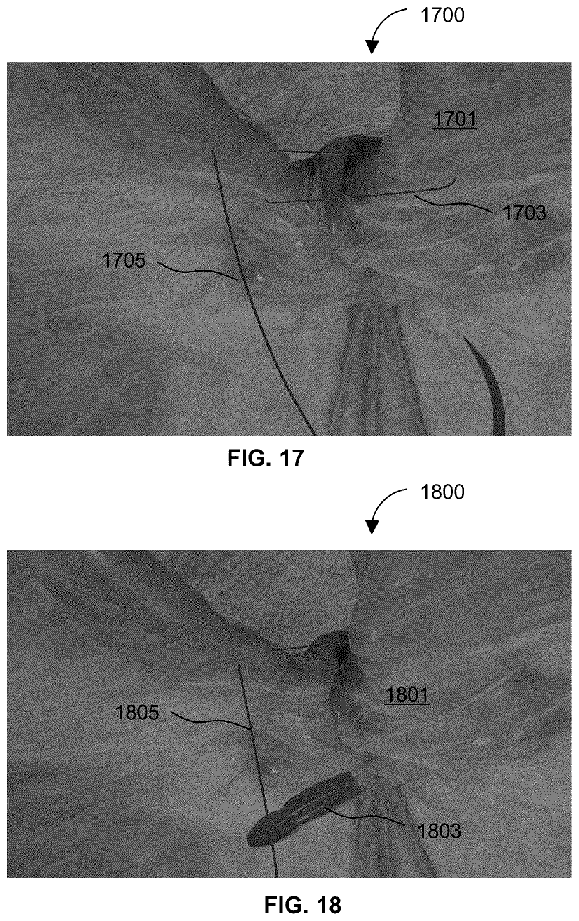

FIG. 17 shows digital content 1700 according to one embodiment of the invention in which simulated sutures 1703 are made in the simulated soft tissue 1701. When pulled under tension, the suture material or thread 1705 has the effect of moving through the soft tissue ‘holes’, i.e. areas at which the tissue has been pierced. It may be pulled through the tissue in either direction, and also when being manipulated by multiple instruments simultaneously. When sutures have been made, the thread may be tightened either by pulling the thread at its loose end (usually attached to a needle) or by individually grabbing and applying tension to each suture. As a result, systems and methods of the invention provide realistic simulation of, for example, the interaction with the thread and the soft tissue, with the soft tissue and the surgical instruments, and with the thread and the surgical instruments.

FIG. 18 shows digital content 1800, according to one embodiment, of thread 1805 being tightened in the simulated surgical suturing of the invention. The custom interaction between the thread 1805 and soft tissue 1801 means that this action enables closure of a defect in the tissue, or the joining together of separate tissues. Similarly to the soft tissue, the suture thread may also be manipulated with the surgical tools 1803 driven by the user. This includes, in non-limiting examples, grabbing, allowing the user to pick up and attempt to stretch the thread, or pulling the thread through the tissue. The system allows for simulating the plastic behavior of suture thread, for instance the ability to wrap it around a surgical instrument and have it retain its shape as the thread slides off the surgical instruments in its wrapped form. This is an important characteristic for knot-tying.

Further, the system models the interaction between the suture thread and the thread itself. Thus, as discussed in more detail below, the system allows for simulating surgical knot-tying. Knot-tying may be required at the start of the simulation in order to anchor the suture thread in the soft tissue, and again at the end of the simulation when a run of stitches have been made and they must be held in place under tension. The user may also tie knots between two separate threads, and in both cases the resulting knot holds its shape and interacts stably with the soft tissue.

Aspects of the invention disclose a system for providing a simulated soft tissue suturing procedure. The system includes a simulation platform as described herein, configured to communicate and exchange data with one or more hand-held components and one or more wearable displays over a network. The simulation platform includes a non-transitory computer-readable storage medium coupled to a processor and encoded with a computer program. The computer program causes the processor to generate and provide an interactive real-time suturing simulation that simulates multiple interactions between a soft tissue, a suture thread and one or more surgical instruments in a manner that movement and deformation of both the suture thread and the soft tissue are simulated using position based dynamics and collision detection. The position based dynamics may be, for example, as described in Müller, 2006, Position Based Dynamics, 3rd Workshop in VR Inter and Phys Sim, incorporated by reference in its entirety herein. The system provides for novel collision detection and simulation algorithms as described herein.

In some embodiments, the simulation platform may be further configured to provide digital content that includes a plurality of three-dimensional (3D) simulated objects within a simulated surgical environment to be presented to one or more users interacting with the simulated surgical environment via associated hand-held components and wearable displays. The plurality of 3D simulated objects may include one or more of a simulated surgical instrument, a simulated soft tissue, a simulated suturing needle, and a simulated suture thread.

As disclosed herein, in non-limiting examples, the surgical instrument may be an instrument used for cutting, dissecting, holding, grasping, occluding, clamping, retracting, exposing, improving vision, suturing, stapling, suctioning, aspirating, dilating, and probing. For example, in non-limiting examples, the surgical instrument may be an instrument used for cutting, dissecting, holding, grasping, occluding, clamping, retracting, exposing, improving vision, suturing, stapling, suctioning, aspirating, dilating, and probing. In non-limiting examples, the virtual surgical instrument may be a scalpel, a needle driver, a clamp, a clip applier, a surgical stapler, a retractor, a periosteal elevator, a rongeur, a nerve hook, a curette, an awl, a probe, a sagittal saw, a drill, a suture, a hammer, a finger, a laparoscopic instrument, an electrocautery, or a suctioning instrument. In the context of simulated soft tissue suturing, the surgical instrument may be any surgical tool related to soft tissue suturing for example, a needle holder, toothed forceps, fine suturing scissors, and suturing material.

The system provides for simulating any internal or external soft tissue. In non-limiting examples the soft tissue may be any supporting tissue of the body for example, skin, muscle, fat, fibrous tissue, blood vessels, tendons, ligaments, and/or fascia. Soft tissue may include any tissue that surrounds organs, bones, and joints.

The simulated suturing material may be either absorbable or nonabsorbable material, for example, natural or synthetic monofilament, and natural or synthetic braided suture.

Further, the system may monitor the actions of the user, including interaction with at least one of the simulated surgical instrument, the simulated soft tissue, the simulated suturing needle, and the simulated thread based on physical user interaction with an associated hand-held component and define user interactions for each. The system may then determine a type and a degree of haptic feedback to be provided to the at least one user via the associated hand-held component based, at least in part, on the defined interactions, and transmit one or more signals to cause the hand-held component to provide the determined haptic feedback to the user and adjust output of digital content to be presented to the user via the wearable display. Accordingly, the system provides for mimicking the movement of any one of the simulated surgical instrument, the simulated soft tissue, the simulated suturing needle, and the simulated thread based, at least in part on the defined interactions.

In non-limiting examples, the defined interactions may be interaction between the simulated suturing needle and the simulated soft tissue types; interaction between the simulated suturing needle and the simulated suture thread; interaction between the simulated suture thread and the simulated soft tissue; interaction between the user and the simulated suturing needle; interaction between the user and the simulated soft tissue; interaction between the simulated suture thread with itself; interaction between the simulated suture thread and the simulated surgical instrument; interaction between the simulated suturing needle and the simulated surgical instrument; and interaction between the simulated surgical instrument and the simulated soft tissue.

As disclosed herein, the simulation platform may include a simulation engine configured to model the interactions using calculations associated with one or more algorithms comprising at least one of a position-based solver, a collision detection algorithm, a deformable mesh algorithm, and an adaptive mesh algorithm. Accordingly, the simulation engine, for example via the simulation platform may be configured to manipulate one or more of a simulated suture thread mesh, a simulated soft tissue mesh, a simulated suture thread runtime, and/or a simulated soft tissue runtime to model the movement of the simulated suture thread as it is pulled through the simulated soft tissue types. As disclosed herein, the movement and deformation of the simulated suture thread and the simulated soft tissue types are simulated using Position Based Dynamics to model the simulated soft tissue types and the simulated suture thread as a collection of particles controlled by a set of constraints.

Further, as noted above, the interactions may include, in non-limiting examples, one or more of prodding, grabbing, piercing, picking up, stretching, cutting, and pulling, such that the simulated thread and simulated suturing needle are manipulated with simulated surgical instruments controlled by the user.

The interaction of the simulated suture material and the simulated soft tissue types may be any type of interaction. For example, the interaction may enable the simulation of a closure of a defect in the simulated soft tissue types and/or a joining together of separate simulated soft tissue types using one or more simulated suturing techniques. For example, the simulated suturing technique may be simulated continuous suturing and/or simulated interrupted suturing. The system allows for different types of suture such as defect suturing in which two sides of a defect are brought together, and/or patch suturing. The simulated suturing may be deep sutures placed under layers of tissue below the skin, buried sutures applied so that the suture knot is retained within the anatomy, purse-string sutures, and/or subcutaneous sutures.

Because the system allows for a template-free, and/or a free-form interaction with the simulated surgical environment, the simulation platform may be configured to allow the user to choose one or more of a type of simulated suturing procedure, a type of simulated suture, a placement of the simulated suture, a direction of simulated suturing, a dominant hand for simulated suturing, and a type and/or number of simulated surgical instruments to use for the simulated soft-tissue suturing.

The haptic and/or visual feedback to be provided to a given user via an associated hand-held component and wearable display may be computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined.

In other aspects, the invention discloses a method 1600 for providing a simulated soft tissue suturing procedure.

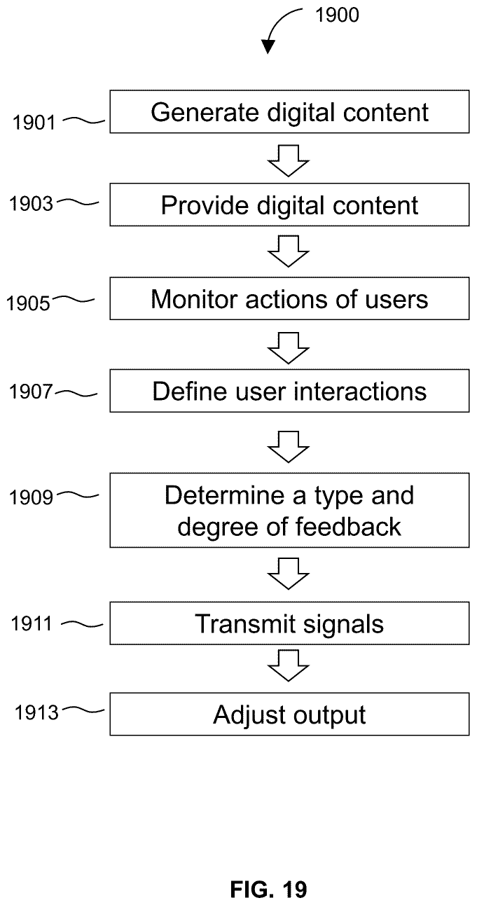

FIG. 19 illustrates a method 1900 for providing a simulated soft tissue suturing procedure according to one embodiment of the invention. The method may include the steps of generating 1901 and providing 1903 via a simulation platform as disclosed herein, digital content comprising a plurality of three-dimensional (3D) simulated objects comprising a simulated surgical instrument, a simulated soft tissue, a simulated suturing needle, and a simulated suture thread within a simulated surgical environment associated with an interactive real-time suturing simulation which may be presented to one or more users interacting with the simulated surgical environment via an associated hand-held component and a wearable display. The interactive real-time suturing simulation may simulate multiple interactions between a soft tissue, a suture thread and one or more surgical instruments in a manner that movement and deformation of both the suture thread and the soft tissue are simulated using position based dynamics and collision detection.

The method may further include monitoring 1905, via the simulation platform, actions of at least one user, including user interaction with at least one of the simulated surgical instrument, the simulated soft tissue, the simulated suturing needle, and the simulated thread based on physical user interaction with an associated hand-held component. The method may include defining 1907, via the simulation platform, user interactions with each. Further, the method may include determining 1909, via the simulation platform, a type and a degree of haptic feedback to be provided to the at least one user via the associated hand-held component based, at least in part, on the defined interactions. The method may include transmitting 1911, via the simulation platform, one or more signals to cause the hand-held component to provide the determined haptic feedback to the user. The method may include adjusting 1913, via the simulation platform, output of digital content to be presented to the user via the wearable display to thereby mimic movement of any one of the simulated surgical instrument, the simulated soft tissue, the simulated suturing needle, and the simulated thread based, at least in part on the defined interactions.

The defined interactions may be one or more of interaction between simulated suturing needle and the simulated soft tissue types; interaction between the simulated suturing needle and the simulated suture thread; interaction between the simulated suture thread and the simulated soft tissue; interaction between the user and the simulated suturing needle; interaction between the user and the simulated soft tissue; interaction between the simulated suture thread with itself; interaction between the simulated suture thread and the simulated surgical instrument; interaction between the simulated suturing needle and the simulated surgical instrument; and interaction between the simulated surgical instrument and the simulated soft tissue.

As disclosed herein, in methods of the invention, the simulation platform may include a simulation engine configured to model the interactions using calculations associated with one or more algorithms comprising at least one of a position-based solver, a collision detection algorithm, a deformable mesh algorithm, and an adaptive mesh algorithm. Accordingly, the simulation engine, for example via the simulation platform may be configured to manipulate one or more of a simulated suture thread mesh, a simulated soft tissue mesh, a simulated suture thread runtime, and/or a simulated soft tissue runtime to model the movement of the simulated suture thread as it is pulled through the simulated soft tissue types. As disclosed herein, the movement and deformation of the simulated suture thread and the simulated soft tissue types are simulated using Position Based Dynamics to model the simulated soft tissue types and the simulated suture thread as a collection of particles controlled by a set of constraints.

Further, as noted above, the interactions may include, in non-limiting examples, one or more of prodding, grabbing, piercing, picking up, stretching, cutting, and pulling, such that the simulated thread and simulated suturing needle are manipulated with simulated surgical instruments controlled by the user.

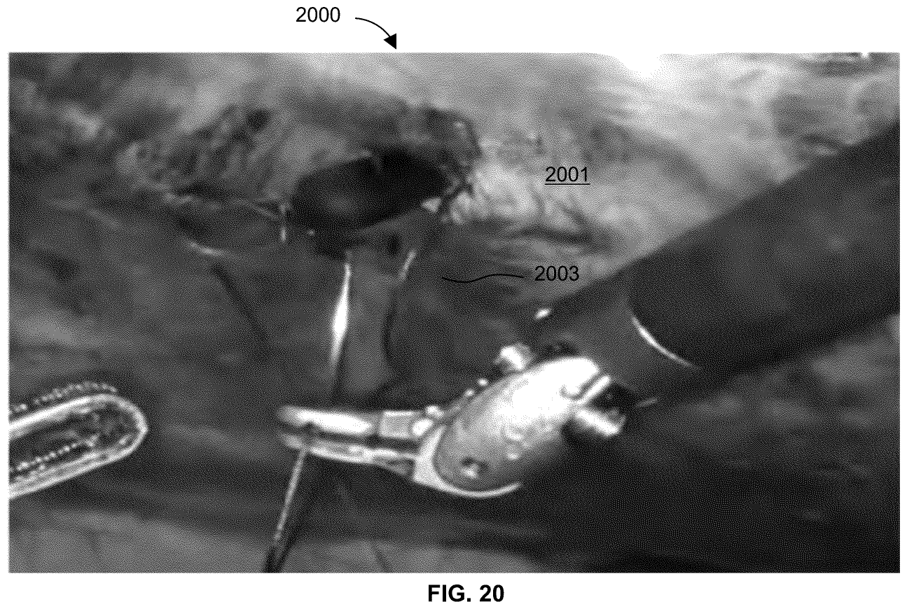

FIG. 20 shows digital content 2000 simulating soft tissue suturing according to one embodiment of the invention. The interaction of the simulated suture material 2003 and the simulated soft tissue 2001 types may be any type of interaction. For example, the interaction may enable the simulation of a closure of a defect in the simulated soft tissue types and/or a joining together of separate simulated soft tissue types using one or more of a simulated continuous suturing and simulated interrupted suturing. Because the system allows for free-form interaction with the simulated surgical environment, the simulation platform may be configured to allow the user to choose one or more of a type of simulated suturing procedure, a type of simulated suture, a placement of the simulated suture, a direction of simulated suturing, a dominant hand for simulated suturing, and a type and/or number of simulated surgical instruments to use for the simulated soft-tissue suturing.

The haptic and/or visual feedback to be provided to a given user via an associated hand-held component and wearable display may be computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined.

### Simulated Surgical Knot-Tying

The invention provides systems and methods for simulated surgical knot-tying. The systems provide for simulating the behavior of suture materials such as suture thread. For example, systems of the invention simulates the ability to wrap the thread around the surgical instrument and have it retain its shape as it is slid off the instrument. This is an important characteristic for surgical knot-tying.

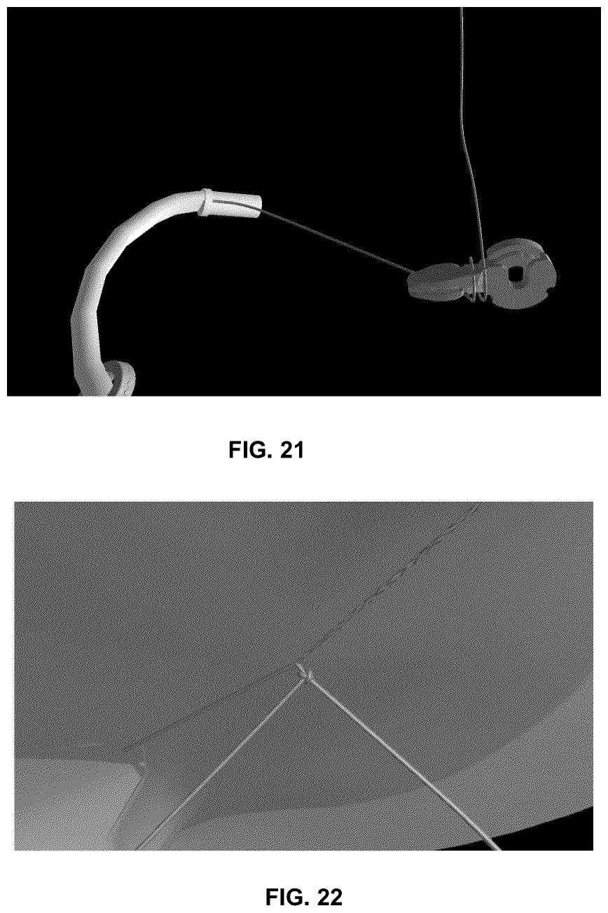

FIG. 21 shows one layer for simulating wrapping suture thread around a surgical instrument in preparation of making a square knot according to one embodiment of the invention. Accordingly, systems and methods of the invention model the interaction between the suture thread itself in order to simulate surgical knot-tying. For example, knot-tying may be required at the start of the simulation in order to anchor the suture thread in the soft tissue, and again at the end of the simulation when a run of stitches has been made and must be held in place under tension. The user can also tie knots between two separate threads, and in both cases the resulting knot holds its shape and interacts stably with the soft tissue.

FIG. 22 shows the underlying construct for the stable completion of simulated knot-tying according to one embodiment of the invention with the suture material being held under pressure as the knot is tightened. Further, the system may be configured to detect when a user is attempting to tie a knot, when that knot has been completed, or when the user has not completed a knot and the thread should unravel.

The user may attempt to suture and tie knots using various techniques, for example those described herein. Further, using the custom algorithms of the system, users may simulate surgical suturing and knot-tying using a different dominant hand or moving in a different direction.

The systems and methods of the invention provide for interactive, real-time simulation of any type of surgical knot-tying, for example square knot-tying using a one-hand or two-hand technique. In specific embodiments, systems of the invention provide the ability to simulate the detailed steps involved in surgical knot-tying, for example, wrapping the suture material around the surgical instrument, grasping the suture thread, and pulling the thread to secure the knot. Systems of the invention provide haptic feedback during this processes to provide a realistic simulation of surgical knot-tying.

Aspects of the invention disclose a system for providing a simulated knot-tying procedure. The system may include a simulation platform, as disclosed herein, configured to communicate and exchange data with one or more hand-held components and one or more wearable displays over a network. The platform may include a non-transitory computer-readable storage medium coupled to the processor and encoded with a computer program. The software may cause the processor to generate and provide an interactive real-time knot-tying simulation that simulates multiple interactions between a soft tissue, a suture thread and one or more surgical instruments in a manner that movement and deformation of both the suture thread and the soft tissue are simulated using position based dynamics and collision detection, such that a component of the simulation includes a model of an interaction between the suture thread and itself.

As described herein, the platform may be configured to provide digital content that includes a plurality of three-dimensional (3D) simulated objects within a simulated surgical environment to be presented to one or more users interacting with the simulated surgical environment via associated hand-held components and wearable displays. The plurality of 3D simulated objects may be a simulated surgical instrument, a simulated soft tissue, a simulated suturing needle, and a simulated suture thread. The digital content may be a choice of simulated knot-tying techniques. In non-limiting examples, the simulated knot-tying techniques may be, for example, a basic knot, square knot, surgeon's knot, deep tie, instrument tie, and/or granny knot.

The platform may be configured to monitor actions of at least one user, including user interaction with at least one of the simulated surgical instrument, the simulated soft tissue, the simulated suturing needle, and the simulated thread based on physical user interaction with an associated hand-held component and define user interactions for each. The system may then determine a type and a degree of haptic feedback to be provided to the at least one user via the associated hand-held component based, at least in part, on the defined interactions. Further, the platform may be configured to transmit one or more signals to cause the hand-held component to provide the determined haptic feedback to the user and further adjust output of digital content to be presented to the user via the wearable display. Accordingly, the system may mimic movement of any one of the simulated surgical instrument, the simulated soft tissue, the simulated suturing needle, and the simulated thread based, at least in part, on the defined interactions.

The defined interactions may be one or more of interaction between simulated suturing needle and the simulated soft tissue types; interaction between the simulated suturing needle and the simulated suture thread; interaction between the simulated suture thread and the simulated soft tissue; interaction between the user and the simulated suturing needle; interaction between the user and the simulated soft tissue; interaction between the simulated suture thread with itself; interaction between the simulated suture thread and the simulated surgical instrument; interaction between the simulated suturing needle and the simulated surgical instrument; and interaction between the simulated surgical instrument and the simulated soft tissue.

Further, the interactions may include one or more of prodding, grabbing, piercing, picking up, stretching, cutting, and pulling, wherein the simulated thread and simulated suturing needle are manipulated with one or more simulated surgical instruments controlled by the user.

The simulation platform may further include a simulation engine, as disclosed herein, configured to model the interactions using calculations associated with one or more algorithms comprising at least one of a position-based solver, a collision detection algorithm, a deformable mesh algorithm, and an adaptive mesh algorithm. The system, via the simulation platform and simulation engine, may be configured to manipulate one or more of a simulated suture thread mesh, a simulated soft tissue mesh, a simulated suture thread runtime, and/or a simulated soft tissue runtime to model the movement of the simulated suture thread as it is pulled through the simulated soft tissue types.

For example, as disclosed herein, the movement and deformation of the simulated suture thread and the simulated soft tissue types may be simulated using Position Based Dynamics to model the simulated soft tissue types and the simulated suture thread as a collection of particles controlled by a set of constraints.

In specific embodiments, the simulated knot-tying procedure includes wrapping the simulated suture thread around a simulated surgical instrument, sliding the wrapped simulated suture thread off of the simulated surgical instrument such that the simulated suture thread retains its wrapped shape once it is disengaged from the simulated surgical instrument.

As noted above, the simulation platform may also be configured to detect when a user is attempting to tie a simulated surgical knot, when the simulated surgical knot has been completed, and/or when the user has not completed a simulated surgical knot. For example, the system may be configured to simulate an unraveling of the simulated surgical knot when the user has not completed a simulated surgical knot.

The haptic and/or visual feedback to be provided to a given user via an associated hand-held component and wearable display may be computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined.

In other aspects, the invention discloses methods for providing a simulated knot-tying procedure.

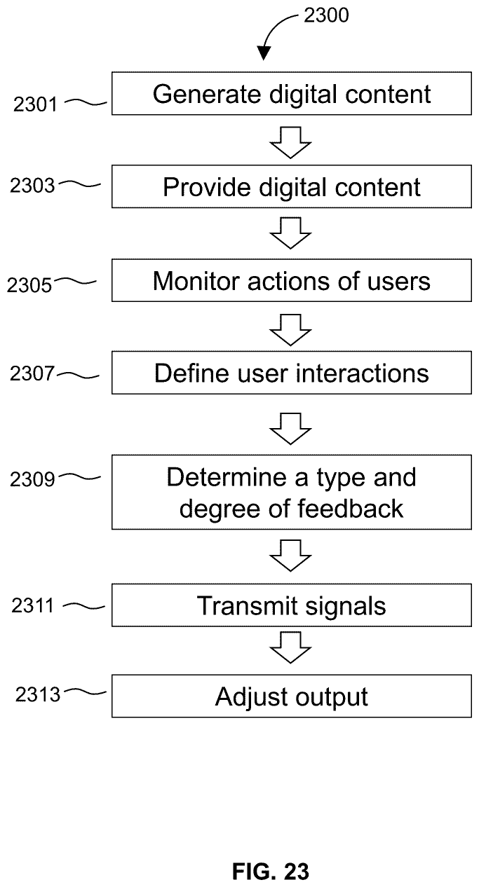

FIG. 23 illustrates a method 2300 for providing a simulated knot-tying procedure according to one embodiment of the invention.

The method may include the steps of generating 2301 and providing 2303, via the simulation platform, digital content comprising a plurality of three-dimensional (3D) simulated objects comprising a simulated surgical instrument, a simulated soft tissue, a simulated suturing needle, and a simulated suture thread within a simulated surgical environment associated with an interactive real-time knot-tying simulation which may be presented to one or more users interacting with the simulated surgical environment via associated hand-held components and wearable displays. The interactive real-time knot-tying simulation may simulate multiple interactions between a soft tissue, a suture thread and one or more surgical instruments in a manner that movement and deformation of both the suture thread and the soft tissue are simulated using position based dynamics and collision detection, such that a component of the simulation includes a model of an interaction between the suture thread and itself.

In some embodiments, the method may further include monitoring 2305, via the simulation platform, actions of at least one user, including user interaction with at least one of the simulated surgical instrument, the simulated soft tissue, the simulated suturing needle, and the simulated thread based on physical user interaction with an associated hand-held component and defining 2307 user interactions for each. Further, the method may include determining 2309, via the simulation platform, a type and a degree of haptic feedback to be provided to the at least one user via the associated hand-held component based, at least in part, on the defined interactions. The method may include then transmitting 2311, via the simulation platform, one or more signals to cause the hand-held component to provide the determined haptic feedback to the user. The method may further include adjusting 2313, via the simulation platform, output of digital content to be presented to the user via the wearable display to thereby mimic movement of any one of the simulated surgical instrument, the simulated soft tissue, the simulated suturing needle, and the simulated thread based, at least in part, on the defined interactions.

As described herein, the platform may be configured to provide digital content that includes a plurality of three-dimensional (3D) simulated objects within a simulated surgical environment to be presented to one or more users interacting with the simulated surgical environment via associated hand-held components and wearable displays. The plurality of 3D simulated objects may be a simulated surgical instrument, a simulated soft tissue, a simulated suturing needle, and a simulated suture thread. The digital content may be a choice of simulated knot-tying techniques. In non-limiting examples, the simulated knot-tying techniques may be, for example, a basic knot, square knot, surgeon's knot, deep tie, instrument tie, and/or granny knot.

The defined interactions may be one or more of interaction between simulated suturing needle and the simulated soft tissue types; interaction between the simulated suturing needle and the simulated suture thread; interaction between the simulated suture thread and the simulated soft tissue; interaction between the user and the simulated suturing needle; interaction between the user and the simulated soft tissue; interaction between the simulated suture thread with itself; interaction between the simulated suture thread and the simulated surgical instrument; interaction between the simulated suturing needle and the simulated surgical instrument; and interaction between the simulated surgical instrument and the simulated soft tissue.

Further, the interactions may include one or more of prodding, grabbing, piercing, picking up, stretching, cutting, and pulling, wherein the simulated thread and simulated suturing needle are manipulated with one or more simulated surgical instruments controlled by the user.

The simulation platform may further include a simulation engine, as disclosed herein, configured to model the interactions using calculations associated with one or more algorithms comprising at least one of a position-based solver, a collision detection algorithm, a deformable mesh algorithm, and an adaptive mesh algorithm. The system, via the simulation platform and simulation engine, may be configured to manipulate one or more of a simulated suture thread mesh, a simulated soft tissue mesh, a simulated suture thread runtime, and/or a simulated soft tissue runtime to model the movement of the simulated suture thread as it is pulled through the simulated soft tissue types.

For example, as disclosed herein, the movement and deformation of the simulated suture thread and the simulated soft tissue types may be simulated using Position Based Dynamics to model the simulated soft tissue types and the simulated suture thread as a collection of particles controlled by a set of constraints.

In specific embodiments of the method, the simulated knot-tying procedure includes wrapping the simulated suture thread around a simulated surgical instrument, sliding the wrapped simulated suture thread off of the simulated surgical instrument such that the simulated suture thread retains its wrapped shape once it is disengaged from the simulated surgical instrument.

As noted above, the simulation platform may also be configured to detect when a user is attempting to tie a simulated surgical knot, when the simulated surgical knot has been completed, and/or when the user has not completed a simulated surgical knot. For example, the system may be configured to simulate an unraveling of the simulated surgical knot when the user has not completed a simulated surgical knot.

The haptic and/or visual feedback to be provided to a given user via an associated hand-held component and wearable display may be computed using an algorithm based on one or more input parameters associated with the hand-held component and the simulated object, a visual effect on the simulated object, an affordance value of a simulated surgical instrument represented by the hand-held component, and a susceptibility value of the simulated object within the simulated surgical environment, wherein the algorithm establishes a resultant effect of the interaction with the digital content that is not pre-determined.

### Digital Twins

Systems and methods of the invention provide for simulating digital twins. Specifically, the invention discloses full replication of a physical device, such as a medical and/or deployment device, within the virtual space with all movement and functionalities replicated. Accordingly, the system creates and delivers a digital twin that allows a user to, for example, learn from the device, validate the device, and/or assist in development of the device. For example, in product development, the invention provides a digital twin from which a user may use in the virtual space to evaluate design issues and/or validate the device, such that any issues may be found in parallel to the development research.

The digital twin may be a virtual model designed to accurately reflect a physical object. For example, the digital twin may be a robotic medical device. For these devices, approval and adoption are dependent on validated training outcomes. Systems and methods of the invention may provide a digital twin of the robotic device such that validation may take place in the simulated surgical environment. Further, the digital twin may be used for training on the medical devices, either robotic or not, such that teams may acquire the necessary skills in the simulated surgical environment to safely adopt the real-world device. As disclosed herein, the systems and methods of the invention provide for remote monitoring/observation of one or more users interacting with the digital twin. Accordingly, systems and methods of the invention provide for skills remote assessment and monitoring, and performance measurement such research and development may be accelerated. Systems and methods of the invention provide for virtual cycles of research and development to inform decisions. Systems and methods of the invention may be used for trial set up and monitoring, certification, application, and competency assessment.

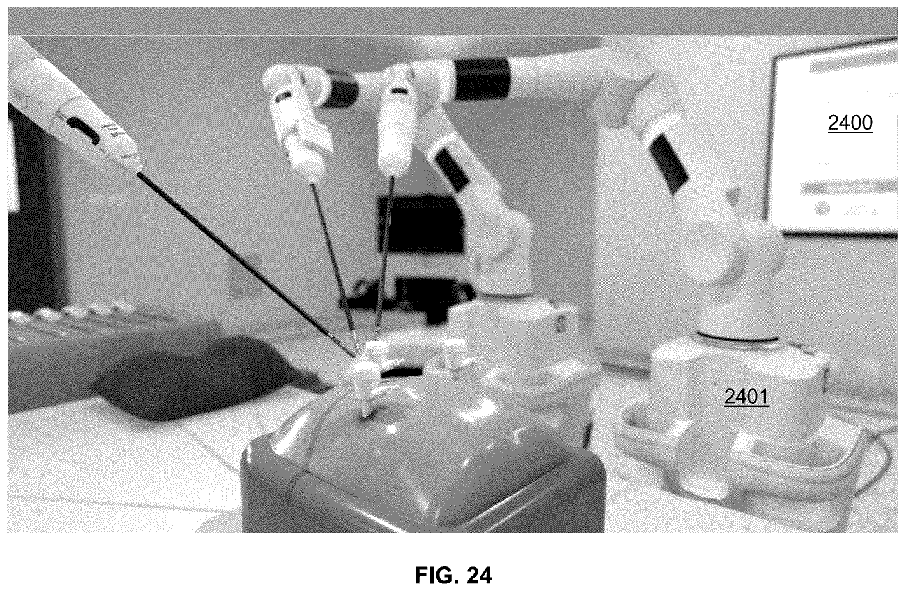

FIG. 24 shows a robotic medical device 2401 interacting with digital content 2400 according to one embodiment of the invention. The system may be configured to provide, for example, data relating to a simulated surgical procedure to a simulated robotic device within a simulated surgical environment such that the robotic device may learn from the simulated surgical procedure. For example, the system may be configured to provide a digital twin of a surgical robot, such as a robotic laparoscopic device, and a simulated surgical environment in which the surgical robot may be trained on the positioning and manipulation of surgical instruments.

Additionally and/or alternatively, the system may be configured to provide a simulated surgical environment to a real-world robotic medical device such that the robotic device may be trained for robotic surgery via the simulated surgical environment. For example, the system may be configured to provide a simulated surgical environment in which a surgical robot may be trained on the positioning and manipulation of surgical instruments such as a robotic laparoscopic device.

Aspects of the invention disclose a system for providing a medical device simulation. The system may include a simulation platform, such as disclosed herein, configured to communicate and exchange date with one or more hand-held components and one or more wearable displays over a network. The platform may comprise a non-transitory computer-readable storage medium coupled to the processor and encoded with a computer program. The computer program may be operable to cause the platform to generate and provide digital content as disclosed herein. The digital content may include at least a simulated medical device in a simulated surgical environment to be presented to one or more users interacting with the simulated surgical environment via associated hand-held components and wearable displays. The simulated medical device may be a digital twin of a real-world medical device such that the platform may be configured to provide a user with highly accurate visualizations of and interaction with the simulated medical device in the simulated surgical environment that match visualizations of and interaction with the real-world medical device in a real-world surgical environment.

Further, the simulation platform may be configured to monitor user actions within the simulated surgical environment. The user interaction may be, for example, at least user interaction with the simulated medical device based on physical user interaction with an associated hand-held component. The simulation platform may be configured to adjust output of digital content to thereby mimic operation and/or movement of the simulated objected.

The simulation platform may be configured to collect data associated with the user's interaction with and operation of the simulated medical device within the simulated surgical environment, as discussed in detail herein. For example, the data collected may be used to validate design and/or performance of the real-world medical device. The data may provide a basis for a redesign of the real-world medical device. The data may provide a risk assessment associated with the operation of the real-world medical device. For example, the data may provide an assessment of safety and risk associated with using the medical device in robot-assisted surgery.

As disclosed in detail herein, the simulation platform may be configured to adapt the digital content in response to the one or more user actions in the virtual surgical environment. The simulation platform may further be configured to track actions of the one or more users; assess a performance of the one or more users based on the tracked actions; determine a performance rating for the actions of the user; and provide feedback to the one or more users associated with their performance rating.

The haptic and/or visual feedback to be provided to a given user via an associated hand-held component and wearable display may be computed using an algorithm based on one or more input parameters associated with the hand-held component and the digital twin and a visual effect on the digital twin within the simulated surgical environment, wherein the algorithm establishes a resultant effect of the interaction with the digital content that is not pre-determined.

In other aspects, the invention discloses a method for providing a medical device simulation.

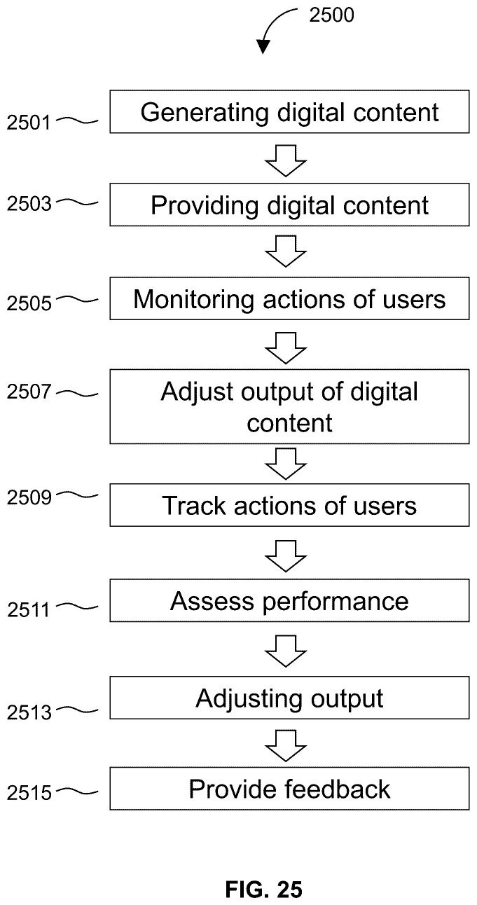

FIG. 25 illustrates one embodiment of a method 2500 for providing a medical device simulation. The method may include generating 2501 and providing 2503, via a simulation platform as disclosed herein, digital content comprising at least a simulated medical device in a simulated surgical environment to be presented to one or more users interacting with the simulated surgical environment via associated hand-held components and wearable displays. The simulated medical device may be a digital twin of a real-world medical device such that the platform may be configured to provide a user with highly accurate visualizations of and interaction with the simulated medical device in the simulated surgical environment that match visualizations of and interaction with the real-world medical device in a real-world surgical environment.

The simulation platform used in the method may be configured to monitor 2505 user actions within the simulated surgical environment. The user interactions may include at least user interaction with the simulated medical device based on physical user interaction with an associated hand-held component. Further, the method may include the simulation platform configured to adjust 2507 output of digital content to thereby mimic operation and/or movement of the simulated objected.

Further, the simulation platform may be configured to collect data associated with the user's interaction with and operation of the simulated medical device within the simulated surgical environment, as discussed in detail herein. For example, the data collected may be used to validate design and/or performance of the real-world medical device. The data may provide a basis for a redesign of the real-world medical device. The data may provide a risk assessment associated with the operation of the real-world medical device. For example, the data may provide an assessment of safety and risk associated with using the medical device in robot-assisted surgery.

As described in detail above, the simulation platform of the method may be configured to adapt the digital content in response to the one or more user actions in the virtual surgical environment. Further, the method may include the simulation platform configured to track 2509 actions of the one or more users; assess 2511 a performance of the one or more users based on the tracked actions; determine 2513 a performance rating for the actions of the user; and provide feedback 2515 to the one or more users associated with their performance rating.

The haptic and/or visual feedback to be provided to a given user via an associated hand-held component and wearable display may be computed using an algorithm based on one or more input parameters associated with the hand-held component and the digital twin and a visual effect on the digital twin within the simulated surgical environment, wherein the algorithm establishes a resultant effect of the interaction with the digital content that is not pre-determined.

Virtual Imaging within the Simulated Surgical Environment

The invention discloses systems and methods for providing virtual imaging techniques within the simulated surgical environment. Replication of medical imaging techniques within a simulated environment is a challenge within the field of software. In the real world, these medical images are created from the interaction of light, sound or radiation. The invention discloses computational and algorithmic techniques that allow for the inference of a cross section of anatomy which can then be visualized in a variety of ways to give the impression of images produced by imaging techniques such as ultrasound, computational tomography and fluoroscopy.

The invention addresses these challenges to generate an image of a slice of a patient from an arbitrary position controlled by the user, of any dynamic or static anatomy and/or medical instruments. This requires the anatomical structures to be modelled to the level of detail that will illustrate a resultant image. Accordingly, each part of the anatomy may be marked to appear on the output of the slice generation, and may be marked to appear differently according to the type of tissue, for example, bone, muscle, fat, or for non-biological components such as instruments, prostheses, etc.

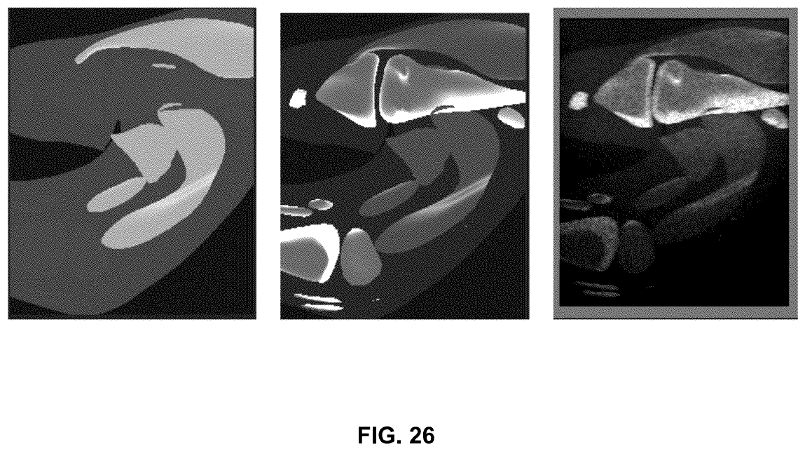

FIG. 26 illustrates generation an imaging slice of a patient from an arbitrary position controlled by the user according to one embodiment of the systems of the invention. In order to generate images in real time, systems and methods of the invention include algorithmic calculations at a shader level. Calculating slices at this level improves the process and makes the simulation possible to run in real time. Several effects are then added to make the final image reflect dynamic, real, medical imaging. The calculations may be performed at the GPU level to keep performance fidelity.

For example, the invention discloses systems and methods for simulating ultrasound sampling in the simulated surgical environment.

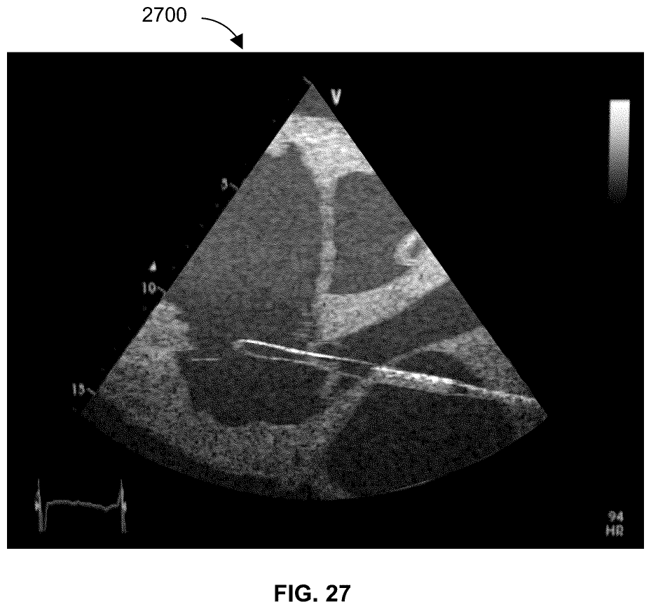

FIG. 27 shows digital content 2700 of a simulated ultrasound image generated within the simulated surgical environment according to one embodiment of the systems and methods of the invention. The systems may use a graphic rendering approach to produce a slice on the defined position of all tissues marked to appear on the final image. Because each tissue can have different properties, the different properties are configured differently to show the differences on the final image, such that the result of a real ultrasound image may be mimicked. The images of the different tissues and instruments are then concatenated to produce an image close to a real ultrasound image. Because this technique be quite noisy in the real world, systems of the invention are configured to apply several layers of post processing to mask the image to look closer to the real technique.

The different steps in this process may be altered to produce different graphic visuals, to thus produce different imaging techniques, or to produce a more diagrammatic image, for example images of anatomy that are similar to those found in a textbook.

To facilitate the imaging of complex objects moving through a patient's internal anatomy, the system provides for deforming any existing 3d model so that it travels along a curved path (i.e., a spline) in 3d space. For example, a completely straight catheter model may be deformed so that it follows the path that the patient's artery takes through their body-allowing this to be observed using an imaging technique such as ultrasound or fluoroscopy. Splines may be deformed with any number of points and in any orientation, using, for example 3D modeling and level design tools such as Unity's standard Transform components, and/or ProBuilder. At runtime, the vertices of each mesh may be individually analyzed and adjusted based on their original position relative to the spline being used to apply the deformations. The result is the overall mesh being deformed to follow the spline without losing its shape, i.e. each vertex is still placed in roughly the same place relative to one another. These calculations may be performed every time the position of the mesh may be adjusted during the operation of the application.

The systems and methods of the invention address the complication of using this approach, which is that some medical devices contain rigid components that need to deform to follow the spline, but otherwise visually appear undeformed or unbent. For example, the motor on a heart pump must not appear bent on any imaging. To solve this, the invention provides systems and methods that selectively lock portions of a mesh so that they deform as a group. This allows them to neatly join with fully deformed vertices on the same mesh, but maintain their original shape exactly. To address performance problems on mobile hardware, additional optimizations are included to ensure that all of the above operates as efficiently as possible. Accordingly, the user experiences a seamless simulation of imaging techniques and renderings within the simulated surgical environment.

Systems and methods of the invention provide for probe control of the imaging device. When operating the imaging technology, it is important that the user's movement of any relevant devices is simulated as accurately as possible so that the resulting imaging output mirrors what would be experienced in real life. For example, as a user operates an ultrasound probe device, the device should move smoothly across the simulated body's 3D mesh in a way that feels natural to the user. For example, the user should not be able to move the device through the patient's body. Systems and methods of the invention include a novel collision detection and resolution system that takes the current position and orientation of the user's hand within VR space and determines the optimal place to position the probe for accurate imaging. This system is capable of accurately simulating and resolving collisions between extensively complex concave meshes, such as a patient's chest area or any other part of their body. For example, the systems of the invention address the simulation of animated meshes, such as a breathing patient, or intravascular imaging with blood flow. Systems of the invention are optimized so performance of the application on mobile hardware is not impacted and feels natural and intuitive to the user of the application.

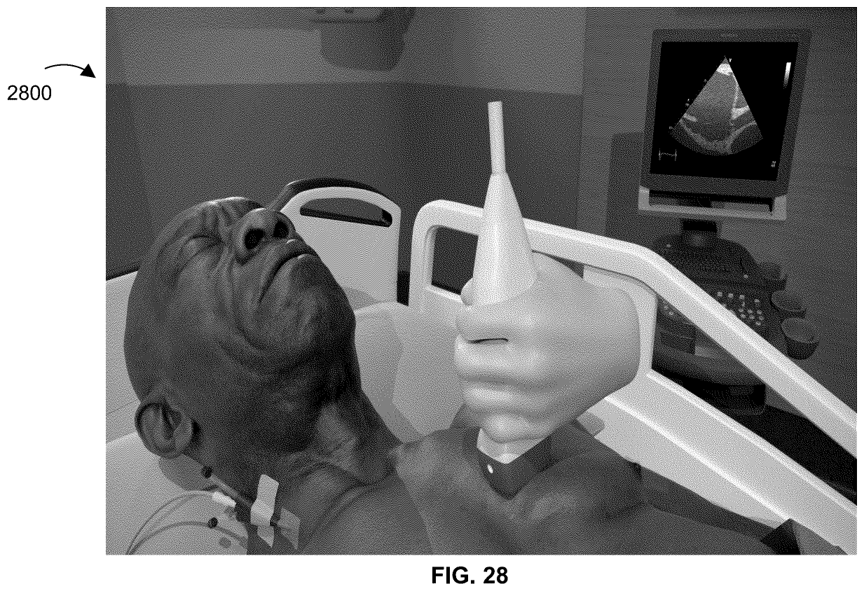

FIG. 28 shows digital content 2800 of a simulated probe device and imaging procedure within the simulated surgical environment, with the simulated image displayed according to one embodiment of the invention.

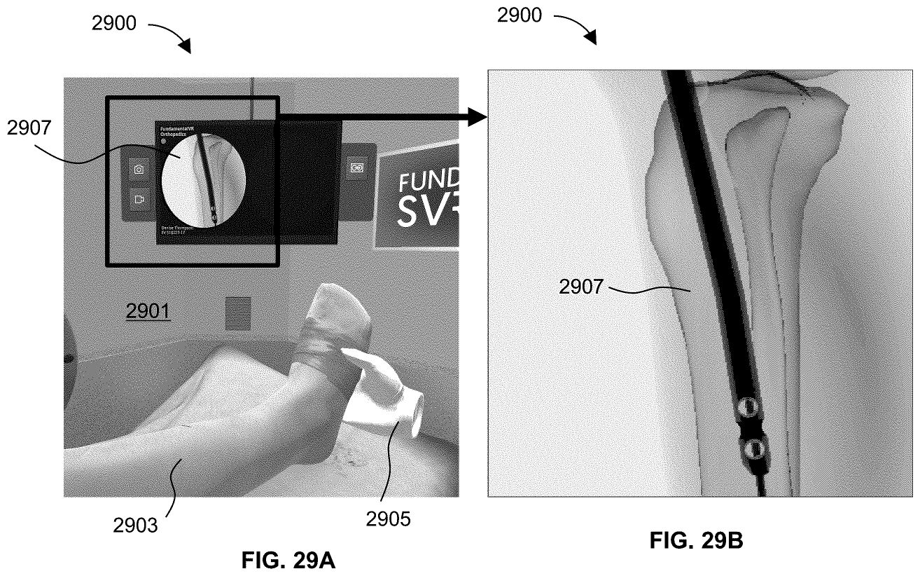

FIGS. 29A and 29B show digital content 2900 of virtual imaging of a virtual patient within the simulated surgical environment, including the virtual image to simulate a section of suprapatellar nailing of tibial fractures from a simulated surgical procedure. FIG. 29A shows the virtual surgical environment 2901 including the patient 2903 and the user 2905 interacting with the patient 2903. The simulated image 2907 is displayed within the simulated surgical environment 2901 and available for interaction with by the user in the simulated medical environment.

FIG. 29B is a cutout of the digital content of the simulated image 2907 in the simulated surgical environment showing a section of suprapatellar nailing of tibial fractures from the simulated surgical procedure

Through the use of layered 3D models and cameras set up to render only certain layers, the systems and methods of the invention may provide a simulated imaging device/machine. For example, systems and methods of the invention provide for a real-time fluoroscopy machine. In addition to the usual surface layer rendering of the patient's skin to the user's eyes, the systems program the fluoroscopy camera to ignore this layer and instead render a series of semi-transparent meshes that represented bones, flesh and metallic objects as they would appear to the real life equivalent equipment. Similarly, where a C-Arm in real life would render what it detected to a screen within the OR, a simulated x-ray camera provided by systems and methods of the invention may be rendered to a screen model within the simulated surgical environment, for example the fluoroscopy image as shown in FIG. 26B. The user is able to interact with, and move or manipulate, the patient at any point. Accordingly, systems and methods of the invention provide for an accurate reproduction of the movability of the patient and camera, as well as a realistic reproduction of the equipment in use.

In aspects, the invention discloses a system for providing simulated medical imaging within a simulated medical environment. The system, as disclosed herein, may include a simulation platform configured to communicate and exchange data with one or more hand-held components and one or more wearable displays over a network. The platform, as disclosed herein, may include a non-transitory computer-readable storage medium coupled to the processor and encoded with a computer program. As disclosed herein, the computer program may be operable to cause the platform to generate and provide a simulated medical environment that comprises a simulated anatomical structure and a simulated medical imaging device. The simulated medical environment may be configured for a user to interact with the simulated medical imaging device to virtually image the simulated anatomical structure and generate a simulated medical image of the simulated anatomical structure from the simulated medical imaging device.

The imaging device may be any device capable of, for example, in non-limiting examples, computed tomography (CT) scan, positron emission tomography (PET), PET/CT scan, magnetic resonance imaging (MRI), ultrasound, fluoroscopy, x-ray, arthrogram, vascular interventional radiography, sonography, and/or mammography. The simulated anatomical structure may be any static or moving anatomical structure.

The simulation platform, as disclosed herein, may be further configured to generate and provide digital content comprising at least a simulated medical imaging device and a simulated anatomical structure in a simulated medical environment which may be presented to one or more users interacting with the simulated medical environment via associated hand-held components and wearable displays. The simulation platform may be configured to monitor user actions within the simulated medical environment. The user interactions may be at least user interaction with the simulated medical imaging device relative to the simulated anatomical structure based on physical user interaction with an associated hand-held component. The simulation platform may be configured to generate, in response to user interaction with the simulated medical imaging device, a simulated medical image of the simulated anatomical structure obtained from the simulated imaging device. The simulated medical image may be displayed in the simulated surgical environment, such that the user may interact with the simulated medical image within the simulated surgical environment.

The simulated medical image may include one or more of a dynamic depiction of the simulated anatomical structure, a static depiction of the simulated anatomical structure, a dynamic depiction of fluid flow within the anatomical structure, and a simulated medical instrument in the simulated medical environment.

Generating the simulated medical image may include generating a two-dimensional (2D) slice of the simulated anatomical structure based on a position of the simulated imaging device relative to the simulated anatomical structure. For example, generating the 2D slice of simulated anatomical structure may include applying one or more anatomical landmarks to the simulated anatomical structure; applying one or more haptic values to the simulated anatomical structure; and applying a templating algorithm. The templating algorithm may recognize and tag the simulated anatomical structure in a two-dimensional view to parameterize the simulated anatomical structure to generate the simulated medical image as a view represented by the simulated medical imaging device. The anatomical landmarks may appear on an output of the generated 2D slice, such that the anatomical landmarks appear differently according to a type of tissue associated with the simulated medical image and/or a non-biological component present in the simulated medical image.

Generating the 2D slice of the simulated anatomical structure may further include performing calculations at a shader level of the simulated medical image; and adding one or more effects on to the calculations to generate the simulated medical image.

In some embodiments, the simulation platform may be configured to generate a simulated dynamic image of a complex simulated medical instrument moving through a simulated patient's internal anatomy. For example, the complex simulated medical instrument may be an intravascular catheter moving through simulated vasculature of a patient such that dynamic blood flow may be simulated as part of the simulated imaging. Further, the simulation platform may be configured to use a spline to apply deformations to a three-dimensional (3D) simulated medical instrument such that the simulated medical instrument travels along a curved path in 3D space. The simulation platform may calculate and adjust vertices of a mesh associated with the simulated medical instrument based on the vertices' original positions relative to the spline used to apply the deformations, such that the calculations are performed each time the position of the mesh is adjusted during operation of the simulated medical instrument. The simulation platform may selectively lock portions of the mesh to allow the selected portions to deform as a group.

The simulation platform may be configured to generate a simulated ultrasound image by generating a two-dimensional (2D) slice based on a defined position of all simulated tissues marked to appear on the simulated ultrasound image, concatenating a plurality of images corresponding to one or more types of simulated tissues and simulated surgical instruments, and applying one or more layers of a post processing.

The simulation platform may be configured to run a collision detection and/or a resolution algorithm. The algorithm may calculate a current position and an orientation of the user's hand within the simulated medical environment to determine an optimal position of a simulated imaging device probe.

Aspects of the invention include a method for providing simulated medical imaging within a simulated medical environment.

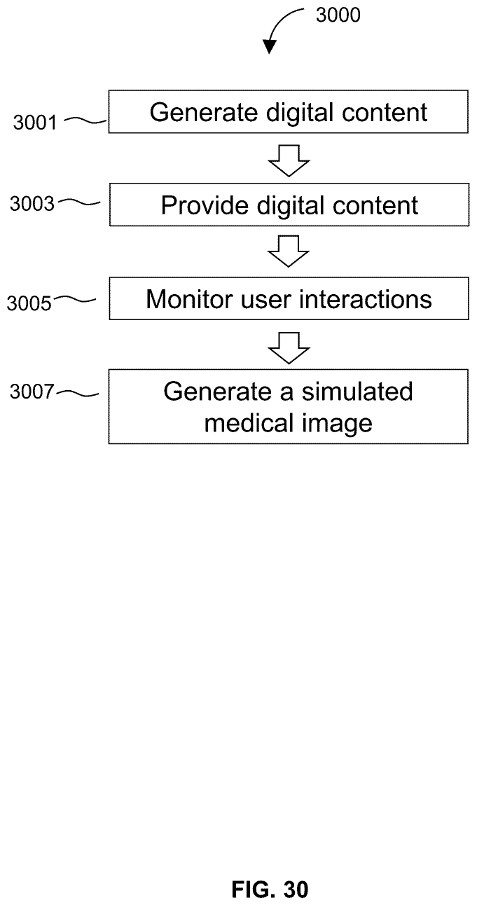

FIG. 30 illustrates a method 3000 for providing simulated medical imaging according to one embodiment of the invention. The method may include generating 3001 and providing 3003, via a simulation platform as disclosed herein, digital content comprising a simulated medical environment that comprises a simulated anatomical structure and a simulated medical imaging device. The simulated medical environment may be configured for a user to interact with the simulated medical imaging device to virtually image the simulated anatomical structure via associated hand-held components and wearable displays and to generate a simulated medical image of the simulated anatomical structure from the simulated medical imaging device.

The method may further comprise monitoring 3005, via the simulation platform, user actions within the simulated medical environment. The user interactions may include at least user interaction with the simulated medical imaging device relative to the simulated anatomical structure based on physical user interaction with an associated hand-held component. The method may include generating 3007, via the simulation platform and in response to user interaction with the simulated medical imaging device, a simulated medical image of the simulated anatomical structure obtained from the simulated imaging device.

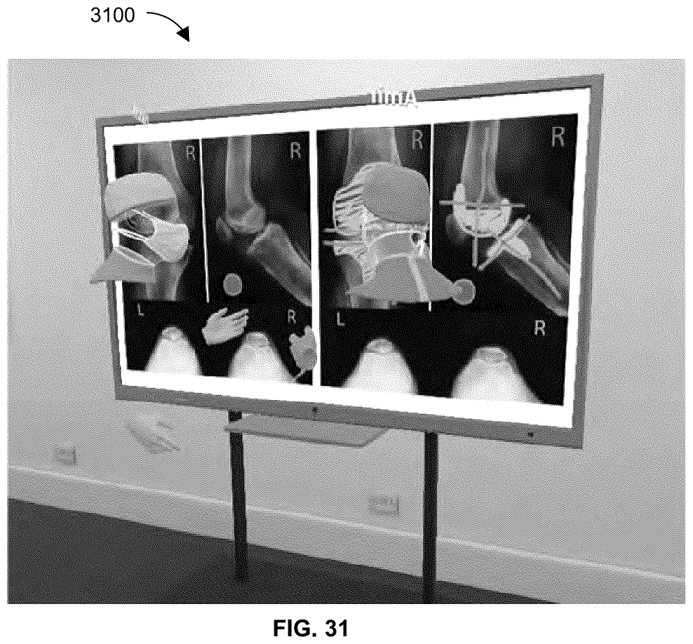

FIG. 31 shows digital content 3100 according to one embodiment of the systems of the invention in which users interact with digital images within the simulated surgical environment.

The imaging device may be any device capable of, for example, in non-limiting examples, computed tomography (CT) scan, positron emission tomography (PET), PET/CT scan, magnetic resonance imaging (MRI), ultrasound, fluoroscopy, x-ray, arthrogram, vascular interventional radiography, sonography, and/or mammography. The simulated anatomical structure may be any static or moving anatomical structure.

The simulated medical image of the method may include one or more of a dynamic depiction of the simulated anatomical structure, a static depiction of the simulated anatomical structure, a dynamic depiction of fluid flow within the anatomical structure, and a simulated medical instrument in the simulated medical environment. Generating the simulated medical image may include generating a two-dimensional (2D) slice of the simulated anatomical structure based on a position of the simulated imaging device relative to the simulated anatomical structure. Generating the 2D slice of the simulated anatomical structure may include applying one or more anatomical landmarks to the simulated anatomical structure; applying one or more haptic values to the simulated anatomical structure; applying a templating algorithm, wherein the templating algorithm recognizes and tags the simulated anatomical structure in a two-dimensional view to parameterize the simulated anatomical structure to generate the simulated medical image as a view represented by the simulated medical imaging device.

In some embodiments, the anatomical landmarks appear on an output of the generated 2D slice, such that the anatomical landmarks appear differently according to a type of tissue associated with the simulated medical image and/or a non-biological component present in the simulated medical image.

In some embodiments of the method, generating the 2D slice of the simulated anatomical structure further includes performing calculations at a shader level of the simulated medical image, and adding one or more effects on to the calculations to generate the simulated medical image. Further, the simulation platform may be configured to generate a simulated dynamic image of a complex simulated medical instrument moving through a simulated patient's internal anatomy. For example, the complex simulated medical instrument may be an intravascular catheter moving through simulated vasculature of a patient such that dynamic blood flow may be simulated as part of the simulated imaging. Further, the simulation platform may be configured to use a spline to apply deformations to a three-dimensional (3D) simulated medical instrument such that the simulated medical instrument travels along a curved path in 3D space. The simulation platform may calculate and adjust vertices of a mesh associated with the simulated medical instrument based on the vertices' original positions relative to the spline used to apply the deformations, such that the calculations are performed each time the position of the mesh is adjusted during operation of the simulated medical instrument. The simulation platform may selectively lock portions of the mesh to allow the selected portions to deform as a group.

The simulation platform may be configured to generate a simulated ultrasound image by generating a two-dimensional (2D) slice based on a defined position of all simulated tissues marked to appear on the simulated ultrasound image, concatenating a plurality of images corresponding to one or more types of simulated tissues and simulated surgical instruments, and applying one or more layers of a post processing.

The simulation platform may be configured to run a collision detection and/or a resolution algorithm. The algorithm may calculate a current position and an orientation of the user's hand within the simulated medical environment to determine an optimal position of a simulated imaging device probe.

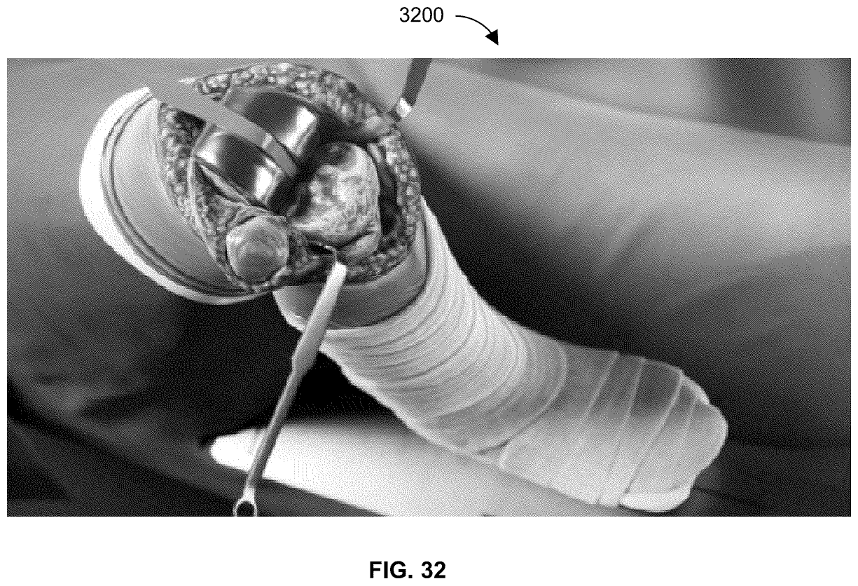

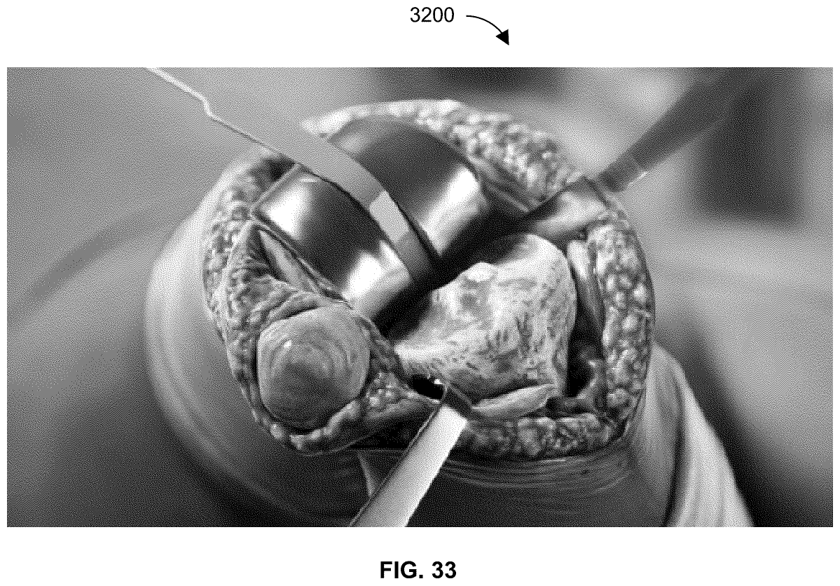

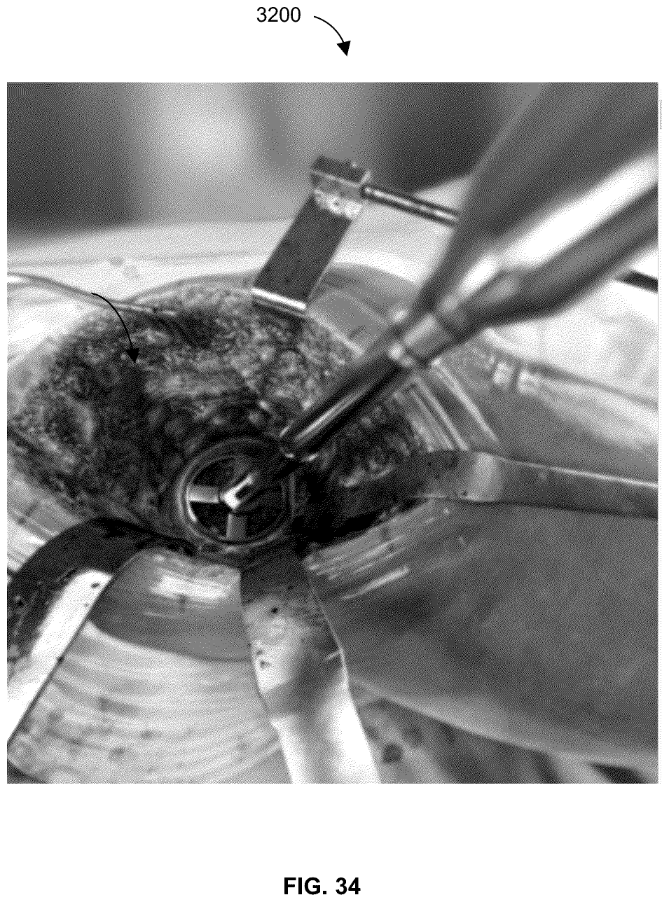

FIG. 32, FIG. 33, and FIG. 34 show digital content 3200 provided according to embodiments of systems and methods of the invention. Specifically, the digital content provides for high fidelity interaction within the simulated surgical environment wherein the users are engaging in a knee replacement.

Simulated Software within the Simulated Surgical Environment

The invention provides systems and methods for interacting with virtual software in the virtual medical environment, for example, software that is used to operate a medical device or machine.

In one aspect, the invention discloses a system for providing simulated software and interaction therewith within a simulated medical environment. The system includes a simulation platform as described herein. For example, the simulation platform may be configured to communicate and exchange data with one or more hand-held components and one or more wearable displays over a network, wherein the platform comprises a non-transitory computer-readable storage medium coupled to the processor and encoded with a computer program. The computer program is operable to cause the platform to generate and provide a simulated medical environment that comprises a simulated device running simulated medical software thereon that is reflective of a real-world version of the medical software. The simulated medical environment may be configured for a user to interact with a simulated user interface associated with the simulated medical software running on the simulated device via one or more hand-held components and a wearable display.

Thus, user interaction with the simulated user interface allows for user operation of and interaction with the simulated device and/or user operation of and interaction with one more other simulated devices within the simulated medical environment. The simulated device and the one or more other simulated device may be any device. For example, the simulated devices may be selected from the group consisting of a computing device and a medical device. The medical device may be any device used within a medical environment or for a home use, for example in non-limiting examples, a patient monitoring machine, a medical imaging machine, an anesthesia machine, an EKG/ECG machine, a surgical treatment machine, a patient monitoring machine, a hemodialysis machine, or an operating room monitoring device. The computing device may be any computing device, for example in non-limiting examples, a personal computer (PC), a tablet, or smartphone.

The simulated medical software may be any medical software reflective of a real-world version. In non-limiting examples, the simulated medical software may be a medical imaging software, a medical drug delivery software, a medical diagnostic software, a medical therapy software, and a patient monitoring software.

In some embodiments, the simulated medical software and the simulated user interface associated with the simulated device are digital twins of a corresponding real-world version of the medical software and a corresponding real-world user interface associated with a real-world device, respectively. Accordingly, the platform may be configured to provide a user with highly accurate visualizations of and interaction with the associated simulated user interface in the simulated medical environment that match visualizations of and interaction with a real-world user interface of a real-world device in a real-world medical environment. In this way, systems of the invention may be used for the design, development, and validation of complex custom software, for example, imaging software associate with a medical imaging device. Users may operate the virtual software associated with a virtual medical device within the simulated medical environment.

Accordingly, in some embodiments, the platform may be configured to collect data associated with user interaction with the simulated user interface associated with the simulated medical software to operate the simulated device within the simulated medical environment. The collected data may then be used to validate the design and/or performance of the real-world version of the medical software.

The simulation platform may be further configured to generate and provide digital content comprising at least a simulated device running simulated medical software thereon and a simulated user interface associated with the simulated medical software in a simulated medical environment; monitor user actions within the simulated medical environment, wherein said user interactions comprise at least user interactions with the simulated user interface based on physical user interaction with one or more associated hand-held components; and generate, in response to user interaction with the simulated user interface, user input with the simulated user interface and operation of the simulated device in response thereto.

In other aspects, the invention discloses methods for providing simulated software and interaction therewith within a simulated medical environment.

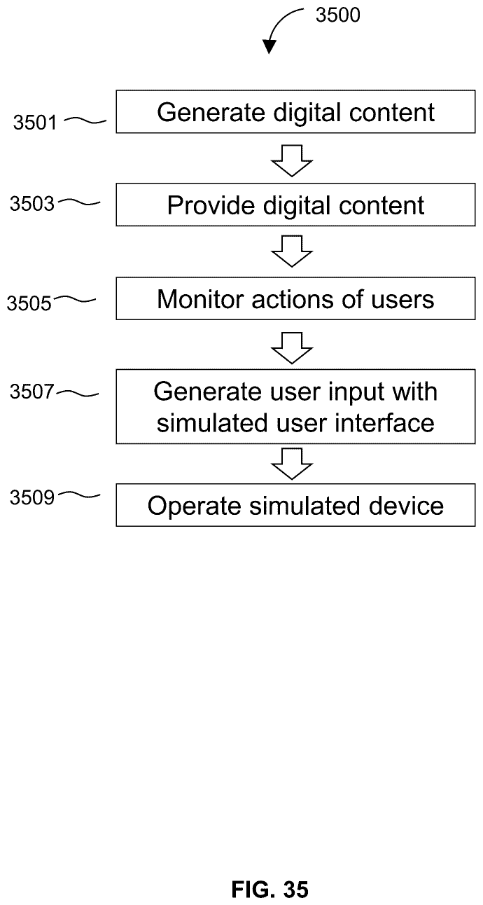

FIG. 35 illustrates one embodiment of a method 3500 for providing simulated software and interaction therewith within a simulated medical environment. The method may include the steps of generating 3501 and providing 3503, via a simulation platform, a simulated medical environment that comprises a simulated device running simulated medical software thereon that is reflective of a real-world version of the medical software, wherein the simulated medical environment is configured for a user to interact with a simulated user interface associated with the simulated medical software running on the simulated device via one or more hand-held components and a wearable display.

Thus, user interaction with the simulated user interface allows for user operation of and interaction with the simulated device and/or user operation of and interaction with one more other simulated devices within the simulated medical environment. The simulated device and the one or more other simulated device may be any device. For example, the simulated devices may be selected from the group consisting of a computing device and a medical device. The medical device may be any device used within a medical environment or for a home use, for example in non-limiting examples, a patient monitoring machine, a medical imaging machine, an anesthesia machine, an EKG/ECG machine, a surgical treatment machine, a patient monitoring machine, a hemodialysis machine, or an operating room monitoring device. The computing device may be any computing device, for example in non-limiting examples, a personal computer (PC), a tablet, or smartphone.

The simulated medical software may be any medical software reflective of a real-world version. In non-limiting examples, the simulated medical software may be a medical imaging software, a medical drug delivery software, a medical diagnostic software, a medical therapy software, and a patient monitoring software.

In some embodiments of the methods, the simulated medical software and the simulated user interface associated with the simulated device are digital twins of a corresponding real-world version of the medical software and a corresponding real-world user interface associated with a real-world device, respectively. Accordingly, the platform may be configured to provide a user with highly accurate visualizations of and interaction with the associated simulated user interface in the simulated medical environment that match visualizations of and interaction with a real-world user interface of a real-world device in a real-world medical environment. In this way, systems of the invention may be used for the design, development, and validation of complex custom software, for example, imaging software associate with a medical imaging device. Users may operate the virtual software associated with a virtual medical device within the simulated medical environment.

Accordingly, in some embodiments of the method, the platform may be configured to collect data associated with user interaction with the simulated user interface associated with the simulated medical software to operate the simulated device within the simulated medical environment. The collected data may then be used to validate the design and/or performance of the real-world version of the medical software.

The simulation platform may be further configured to generate and provide digital content comprising at least a simulated device running simulated medical software thereon and a simulated user interface associated with the simulated medical software in a simulated medical environment. In some embodiments, the methods include monitoring 3505 user actions within the simulated medical environment, wherein said user interactions comprise at least user interactions with the simulated user interface based on physical user interaction with one or more associated hand-held components; and generate 3507, in response to user interaction with the simulated user interface, user input with the simulated user interface and operation 3509 of the simulated device in response thereto.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

“Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1.-144. (canceled)

145. A system for providing simulated software and interaction therewith within a simulated medical environment, the system comprising:
a simulation platform configured to communicate and exchange data with one or more hand-held components and one or more wearable displays over a network, wherein the platform comprises a non-transitory computer-readable storage medium coupled to the processor and encoded with a computer program operable to cause the platform to:
generate and provide a simulated medical environment that comprises a simulated device running simulated medical software thereon that is reflective of a real-world version of the medical software, wherein the simulated medical environment is configured for a user to interact with a simulated user interface associated with the simulated medical software running on the simulated device via one or more hand-held components and a wearable display.

146. The system of claim 145, wherein user interaction with the simulated user interface allows for user operation of and interaction with the simulated device and/or user operation of and interaction with one more other simulated devices within the simulated medical environment.

147. The system of claim 146, wherein the simulated device and the one or more other simulated devices are selected from the group consisting of a computing device and a medical device.

148. The system of claim 147, wherein the computing device is selected from the group consisting of a personal computer (PC), a tablet, and a smartphone.

149. The system of claim 147, wherein the medical device is selected from the group consisting of a medical imaging machine, an anesthesia machine, an EKG/ECG machine, a surgical treatment machine, a patient monitoring machine, a hemodialysis machine, and one or more operating room monitoring devices.

150. The system of claim 146, wherein the simulated medical software and the simulated user interface associated with the simulated device are digital twins of corresponding real-world version of the medical software and a real-world user interface associated with a real-world device, respectively, such that the platform is configured to provide a user with highly accurate visualizations of and interaction with the associated simulated user interface in the simulated medical environment that match visualizations of and interaction with a real-world user interface of a real-world device in a real-world medical environment.

151. The system of claim 145, wherein the platform is configured to collect data associated with user interaction with the simulated user interface associated with the simulated medical software to operate the simulated device within the simulated medical environment.

152. The system of claim 151, wherein the data collected is used to validate the design and/or performance of the real-world version of the medical software.

153. The system of claim 145, wherein the simulation platform is further configured to:
generate and provide digital content comprising at least a simulated device running simulated medical software thereon and a simulated user interface associated with the simulated medical software in a simulated medical environment;
monitor user actions within the simulated medical environment, wherein said user interactions comprise at least user interactions with the simulated user interface based on physical user interaction with one or more associated hand-held components; and
generate, in response to user interaction with the simulated user interface, user input with the simulated user interface and operation of the simulated device in response thereto.

154. The system of claim 145, wherein the simulated medical software reflective of a real-world version of the medical software is selected from the group consisting of a medical imaging software, a medical drug delivery software, a medical diagnostic software, a medical therapy software, and a patient monitoring software.

155. A method for providing simulated software and interaction therewith within a simulated medical environment, the method comprising:
generating and providing, via a simulation platform, a simulated medical environment that comprises a simulated device running simulated medical software thereon that is reflective of a real-world version of the medical software, wherein the simulated medical environment is configured for a user to interact with a simulated user interface associated with the simulated medical software running on the simulated device via one or more hand-held components and a wearable display.

156. The method of claim 155, wherein user interaction with the simulated user interface allows for user operation of and interaction with the simulated device and/or user operation of and interaction with one more other simulated devices within the simulated medical environment.

157. The method of claim 156, wherein the simulated device and the one or more other simulated devices are selected from the group consisting of a computing device and a medical device.

158. The method of claim 155, wherein the computing device is selected from the group consisting of a personal computer (PC), a tablet, and a smartphone.

159. The method of claim 155, wherein the medical device is selected from the group consisting of a medical imaging machine, an anesthesia machine, an EKG/ECG machine, a surgical treatment machine, a patient monitoring machine, a hemodialysis machine, and one or more operating room monitoring devices.

160. The method of claim 155, wherein the simulated medical software and the simulated user interface associated with the simulated device are digital twins of corresponding real-world version of the medical software and a real-world user interface associated with a real-world device, respectively, such that the platform is configured to provide a user with highly accurate visualizations of and interaction with the associated simulated user interface in the simulated medical environment that match visualizations of and interaction with a real-world user interface of a real-world device in a real-world medical environment.

161. The method of claim 155, wherein the simulation platform is configured to collect data associated with user interaction with the simulated user interface associated with the simulated medical software to operate the simulated device within the simulated medical environment.

162. The method of claim 161, wherein the data collected is used to validate the design and/or performance of the real-world version of the medical software.

163. The method of claim 155, further comprising:
generating and providing, via the simulation platform, digital content comprising at least a simulated device running simulated medical software thereon and a simulated user interface associated with the simulated medical software in a simulated medical environment;
monitoring, via the simulation platform, user actions within the simulated medical environment, wherein said user interactions comprise at least user interactions with the simulated user interface based on physical user interaction with one or more associated hand-held components; and
generating, via the simulation platform and in response to user interaction with the simulated user interface, user input with the simulated user interface and operation of the simulated device in response thereto.

164. The method of claim 155, wherein the simulated medical software reflective of a real-world version of the medical software is selected from the group consisting of a medical imaging software, a medical drug delivery software, a medical diagnostic software, a medical therapy software, and a patient monitoring software.
